(19) United States
(12) Patent Application Publication
(10) Pub. No.: US 2026/0165966 A1
(43) Pub. Date: Jun. 18, 2026

(54) LIQUID PHARMACEUTICAL FORMULATIONS OF LOOP DIURETICS AND METHODS OF ADMINISTERING THE SAME

(71) Applicant: SCPHARMACEUTICALS INC., Burlington, MA (US)

(72) Inventors: Jennifer VANDIVER, Burlington, MA (US); Olatokumbo Olajimi Luca OGUNLEYE, Burlington, MA (US); Michael HASSMAN, Burlington, MA (US); Erik Mark PECORELLI, Burlington, MA (US); John F. MOHR, Burlington, MA (US)

(21) Appl. No.: 19/110,649

(22) PCT Filed: Sep. 15, 2023
(86) PCT No.: PCT/US2023/032872

§ 371 Date: Mar. 11, 2025

Related U.S. Application Data
(60) Provisional application No. 63/376,157, filed on Sep. 19, 2022.

(51) Int. Cl. A61K 9/08; A61K 9/00; A61K 31/635; A61K 47/02; A61K 47/10; A61K 47/18

(52) U.S. Cl. CPC A61K 9/08; A61K 9/0019; A61K 31/635; A61K 47/02; A61K 47/10; A61K 47/18

(57) ABSTRACT

Disclosed herein, in part, are liquid pharmaceutical formulations and unit liquid pharmaceutical formulations comprising a loop diuretic, and a pharmaceutically acceptable excipient. In certain embodiments, the formulations include a buffer and/or an antioxidant. Methods of treating congestion, edema, fluid overload, or hypertension in a patient in need thereof are also provided.

### CROSS-REFERENCE TO RELATED APPLICATION

This application claims priority to and the benefit of U.S. Provisional Patent Application No. 63/376,157, filed on Sep. 19, 2022, the content of which is incorporated herein in its entirety.

### BACKGROUND

Loop diuretics, can be used in the treatment of hypertension, edema and related conditions, including congestive heart failure. For example, furosemide, is commonly used in the treatment and/or management of edema associated with congestive heart failure, hepatic failure and renal disease. H. Bundgaard, T. Norgaard, N. M. Nielsen, “Photodegradation and hydrolysis of furosemide and furosemide esters in aqueous solutions,” International Journal of Pharmaceutics 42, 217 (1988).

Oral bioavailability, and therefore oral efficacy, of loop diuretics, such as furosemide, is often limited. Furosemide is commonly administered both parenterally and orally, although highly variable oral absorption is observed due to the combined effects of limited solubility and decreased stability at acidic pH. B. Devarakonda, D. P. Otto, A. Judefeind, R. A. Hill, M. M. de Villiers, “Effect of pH on the solubility and release of furosemide from polyamidoamine (PAMAM) dendrimer complexes,” International Journal of Pharmaceutics 345, 142 (Dec. 10, 2007). Accordingly, furosemide typically is administered intravenously or intramuscularly for most patients with congestive heart failure or other forms of more advanced edema.

Intravenous administration of a pharmaceutical drug, such as a loop diuretic, requires a trained healthcare professional for placement of the catheter and administration of the drug solution. In contrast, a pharmaceutical drug can be administered by subcutaneous injections or infusions by the patient or caregiver with the aid of auto-injection devices and/or wearable, on-body delivery infusion devices, for example, at home. Subcutaneous administration of loop diuretics by the patient or caregiver also can allow for more optimal therapeutic administration and total dose to provide a more appropriate pharmacokinetic and pharmacodynamic profile and patient outcome.

For subcutaneous administration, discomfort and pain during administration should be minimized so as to avoid poor patient compliance with the treatment regimen. Factors that can contribute to pain and discomfort perceived by a patient upon, during, or after subcutaneous administration include the injection volume, the pH of the formulation, and the osmoticity or tonicity of the formulation. Moreover, such a formulation should be stable in solution so that it readily is available for use and/or can be pre-loaded into a variety of drug delivery devices.

Therefore, a need exists for improved pharmaceutical formulations containing a loop diuretic (e.g., bumetanide, furosemide, or torsemide) that are chemically and physically stable over their shelf-life, contain a sufficient concentration of the loop diuretic (e.g., a therapeutically effective amount in a suitable volume), and are at an appropriate pH and osmolality, for example, to permit subcutaneous administration of the loop diuretic.

### SUMMARY

In one aspect, the invention provides liquid pharmaceutical formulations of a loop diuretic (e.g., bumetanide, furosemide, or torsemide), for example, a loop diuretic, and a pharmaceutically acceptable excipient.

In various embodiments of the invention, the liquid pharmaceutical formulation comprises:
(i) a loop diuretic such as furosemide, or a pharmaceutically acceptable salt thereof, in a therapeutically effective amount;
(ii) an antioxidant; and
(iii) a pharmaceutically acceptable buffer.

In another aspect, the invention provides a unit liquid pharmaceutical formulation of a loop diuretic such as furosemide or a pharmaceutically acceptable salt thereof.

In various embodiments of the invention, the unit liquid pharmaceutical formulation comprises:
(i) from about 1 mg to about 250 mg of furosemide, or a pharmaceutically acceptable salt thereof;
(ii) from about 0.1% (w/w) to about 30% (w/w) of one or more pharmaceutically acceptable excipients selected from the group consisting of ethanol, benzyl alcohol, glycerin, N-methyl-pyrrolidone (NMP), sodium chloride, sodium carbonate, mannitol, lactose, dextrose, a polyethylene glycol (PEG), propylene glycol, a polysorbate, a polyvinylpyrrolidone (PVP), a cyclodextrin or a derivative thereof, vitamin E or a derivative thereof, and any combination thereof;
(iii) from about 0.05% (w/w) to about 2% (w/w) of an antioxidant selected from the group consisting of sodium metabisulfite, sodium bisulfite monothioglycerol, methionine, EDTA, and combinations thereof; and
(iv) from about 10 mM to about 500 mM of a pharmaceutically acceptable buffer selected from the group consisting of histidine, a citrate salt, sodium phosphate, potassium phosphate, tromethamine or a pharmaceutically acceptable salt thereof, and any combination thereof,
wherein the pH of the liquid pharmaceutical formulation is from about 6.5 to about 8.5.

Another aspect of the invention provides a method of treating congestion, edema, fluid overload, or hypertension in a patient in need thereof, the method comprising administering to the patient a liquid pharmaceutical formulation or a unit liquid pharmaceutical formulation described herein.

Another aspect of the invention provides a kit for the treatment of congestion, edema, fluid overload, or hypertension comprising a liquid pharmaceutical formulation, or a unit liquid pharmaceutical formulation described herein and a delivery device (e.g., pump device, autoinjector, etc.), along with written instructions for use.

### DETAILED DESCRIPTION

The invention provides liquid pharmaceutical formulations and unit liquid pharmaceutical formulations containing a loop diuretic (e.g., bumetanide, furosemide, or torsemide), medical kits containing the same, and methods of using the liquid pharmaceutical formulations and unit liquid pharmaceutical formulations to treat medical disorders e.g., congestion, edema, fluid overload, or hypertension in a patient in need thereof. In particular, the invention can provide pharmaceutical formulations of a loop diuretic that include a pharmaceutically acceptable excipient, such as an antioxidant, a pH adjuster, a solubilizing agent, an osmolarity adjuster, and/or a pharmaceutically acceptable buffer, for administration to a patient.

### Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein are constructed according to the standard rules of chemical valency known in the chemical arts.

The terms “a” and “an” as used herein mean “one or more” and include the plural unless the context is inappropriate.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10%, ±5%, ±3%, ±2%, or ±1% variation from the nominal value unless otherwise indicated or inferred from the context.

At various places in the present specification, values are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As used herein, a “compound” (including a specifically named compound, e.g., furosemide, bumetanide, ethacrynic acid, torsemide) refers to the compound itself and its pharmaceutically acceptable salts unless otherwise understood from the context of the description or expressly limited to one particular form of the compound, e.g., the compound itself, or a pharmaceutically acceptable salt thereof.

As used herein, “furosemide” refers to a compound having the formula:

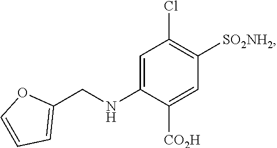 [SMILES: NS(=O)(=O)c1cc(C(=O)O)c(NCc2ccco2)cc1Cl]

and pharmaceutically acceptable salts thereof. Such salts may include, but are not limited to, furosemide sodium salt and furosemide quaternary ammonium salt. Furosemide may be referred to by other names, for example, frusemide, 5-(aminosulphonyl)-4-chloro-2-[(2-furanyl-methyl)amino]benzoic acid, or its IUPAC name, 4-chloro-2-(furan-2-ylmethylamino)-5-sulfamoyl-benzoic acid, or its common trade name, Lasix®.

As used herein, “bumetanide” refers to a compound having the formula:

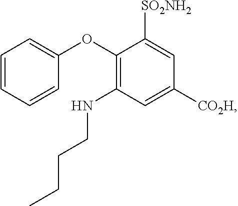 [SMILES: CCCCNc1cc(C(=O)O)cc(S(N)(=O)=O)c1Oc1ccccc1]

and pharmaceutically acceptable salts thereof. Such salts may include, but are not limited to, furosemide potassium salt and furosemide sodium salt. Bumetanide may be referred to by other names, for example, bumedyl, bumethanide, or its IUPAC name, 3-(butylamino)-4-phenoxy-5-sulfamoylbenzoic acid, or its common trade names, Bumex® and Burinex®.

As used herein, “ethacrynic acid” refers to a compound having the formula:

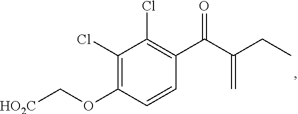 [SMILES: C=C(CC)C(=O)c1ccc(OCC(=O)O)c(Cl)c1Cl]

and pharmaceutically acceptable salts thereof. Such salts may include, but are not limited to, ethacrynic acid sodium salt (also referred to as ethacrynate sodium). Ethacrynic acid may be referred to by other names, for example, etacrynic acid, or its IUPAC name, 2-[2,3-dichloro-4-(2-methylidenebutanoyl) phenoxy]acetic acid, or its common trade names, Sodium Edecrin® and Edecrin®.

As used herein, “torsemide” refers to a compound having the formula:

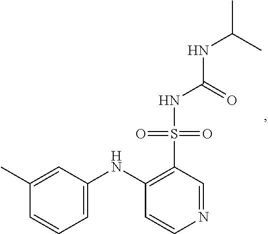 [SMILES: Cc1cccc(Nc2ccncc2S(=O)(=O)NC(=O)NC(C)C)c1]

and pharmaceutically acceptable salts thereof. Such salts may include, but are not limited to, torsemide sodium salt. Torsemide may be referred to by other names, for example, torasemide, 1-isopropyl-3-((4-(3-methylphenylamino)pyridine)-3-sulfonyl) urea, 1-isopropyl-3-((4-m-toluidino-3-pyridyl) sulfonyl) urea, or its IUPAC name, 1-[4-(3-methylanilino)pyridin-3-yl]sulfonyl-3-propan-2-ylurea, or its common trade name, Demadex®.

As used herein, the terms “subject” and “patient” refer to organisms to be treated by the methods and/or compositions described herein. Such organisms are preferably mammals (e.g., murines, simians, equines, bovines, porcines, canines, felines, and the like), and more preferably humans (e.g., adult humans).

As used herein, a “buffer” refers to an aqueous solution that is resistant to changes in pH. A buffer may include a “buffering agent” such as a weak acid and its salt, or a weak base and its salt, which assist in maintaining the stability of the pH. Examples of buffers used in pharmaceutical formulations include bicarbonate buffers, carbonate buffers, citrate buffers, histidine buffers, phosphate buffers, tartrate buffers, tris(hydroxymethyl)aminomethane (or 2-amino-2-hydroxymethyl-propane-1,3-diol[(HOCH₂)₃CNH₂]) buffers, and combinations thereof. Certain of these buffers are suitable for pharmaceutical formulations administered subcutaneously.

Tris(hydroxymethyl)aminomethane or a tris(hydroxymethyl)aminomethane buffer can be referred to as “TRIS,” “Tris,” “Tris buffer,” “Trisamine,” “THAM,” “tromethamine,” and other names. In addition, many buffers and/or buffer systems can include Tris, or a pharmaceutically acceptable salt thereof, and can be used in the present teachings. For example, Tris-buffered saline (“TBS”), Tris-hydrochloride buffer (“Tris-HCl”), Tris base (pH 10.6), Tris/borate/ethylene diamine tetra-acetate (“EDTA”) buffer (“TBE”), and Tris/acetate/EDTA buffer (“TAE”). Tris base often is used with Tris-HCl to prepare Tris buffers at a desired pH. In addition, the present teachings can include Tris-related compounds, for example, compounds derived from Tris or structurally related to Tris, that can act as a buffer.

As used herein, “tonicity” refers to the ionic strength or concentration of ions in a solution such as a pharmaceutical formulation. Tonicity often is measured in molarity (“M”). As used herein, an “isotonic solution,” an “isotonic formulation,” an “isotonic pharmaceutical formulation,” and a pharmaceutical formulation that is “isotonic” refers to a solution or formulation that has the same or similar concentration of ions as found in bodily fluids. As used herein, “physiological pH” refers to a pH of about 7.4.

As used herein, “osmoticity” and “osmolality” refer to the osmotic pressure of a solution such as a pharmaceutical formulation. Osmoticity often is measured in osmolarity (“Osm/L” or “OsM”) or osmolality (“Osm/kg”), which can be used interchangeably herein. When measuring freezing point depression, the observed value is the osmolality of the solution. In contrast to tonicity, osmoticity accounts for un-ionized solutes in a solution such that when present, the osmolarity or osmolality of the solution will be higher than its tonicity. The osmolarity of a liquid pharmaceutical formulation described herein can be measured, for example, using a vapor pressure method.

As used herein, “osmolarity adjustor” and “osmotic agent” refer to a pharmaceutically acceptable compound that may be added to a liquid pharmaceutical formulation described herein in order to modulate the osmolarity of the liquid pharmaceutical formulation.

As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. In certain embodiments, supplementary active ingredients can also be incorporated into the pharmaceutical compositions.

As used herein, “pharmaceutically acceptable excipient” refers to a substance that aids the administration of an active agent to and/or its absorption by a subject and can be included in the compositions and formulations of the present invention without causing a significant adverse toxicological effect on the patient. In certain embodiments, a pharmaceutically acceptable excipient described herein may also be used to improve the stability of a liquid pharmaceutical formulation or a unit liquid pharmaceutical formulation described herein. In certain embodiments, a pharmaceutically acceptable excipient described herein may also be used to improve the solubility of a loop diuretic in a liquid pharmaceutical formulation or a unit liquid pharmaceutical formulation described herein. Non-limiting examples of pharmaceutically acceptable excipients include liquid vehicles such as water, NaCl, normal saline solutions, buffers and buffering agents such as a phosphate buffered saline solution, components or compounds of emulsions (e.g., such as an oil/water or water/oil emulsions), lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxypropylmethylcellulose, polyvinyl pyrrolidine, and colors, and the like. Such preparations can be sterilized and, if desired, mixed with auxiliary excipients such as lubricants, preservatives, stabilizers, solubilizing agents, wetting agents, emulsifiers, salts for influencing osmotic pressure, buffers, coloring, and/or aromatic substances and the like that do not deleteriously react with the compounds of the invention. For examples of excipients and carriers, see Martin, Remington's Pharmaceutical Sciences, 15th Ed., Mack Publ. Co., Easton, PA (1975).

As used herein, the term “pharmaceutically acceptable carrier” refers to any of the standard pharmaceutical carriers, such as a buffer such as a phosphate buffered saline solution or a tromethamine buffered water solution, water (e.g., water for injection (WFI)), emulsions (e.g., such as an oil/water or water/oil emulsions), and various types of wetting agents. The compositions also can include stabilizers and preservatives. For examples of carriers, stabilizers and adjuvants, see Martin, Remington's Pharmaceutical Sciences, 15th Ed., Mack Publ. Co., Easton, PA [1975].

As used herein, the term “pharmaceutically acceptable salt” refers to any pharmaceutically acceptable salt (e.g., acid or base) of a compound of the present invention (e.g., a loop diuretic described herein) which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

It should be understood herein that, unless stated otherwise, all weights described herein correspond to the weight of a compound (e.g., furosemide, torsemide, bumetanide) in its free-acid/free-base form.

As used herein, the term “effective amount” refers to the amount of a composition (e.g., a liquid pharmaceutical formulation or a compound disclosed herein) sufficient to effect beneficial or desired results. An effective amount can be administered in one or more administrations, applications or dosages and is not intended to be limited to a particular formulation or administration route.

As used herein, the terms “treat,” “treating,” and “treatment” include any effect, e.g., lessening, reducing, modulating, ameliorating or eliminating, that results in the improvement of the condition, disease, disorder, and the like, or ameliorating a symptom thereof.

The phrase “therapeutically-effective amount” as used herein means that amount of a composition (e.g., a liquid pharmaceutical formulation or a compound disclosed herein) which is effective for producing some desired therapeutic effect in a subject.

As used herein, the term “congestion” (in heart failure) is the presence of signs and symptoms of extracellular fluid accumulation that results in increased cardiac filling pressures leading to reduced cardiac output. This reduced cardiac output is further exacerbated by neurohormonal activation leading to increased renal sodium and water avidity resulting in an increased plasma volume.

As used herein, “fluid overload,” “volume overload” and “hypervolemia”, may describe a medical condition where there is too much fluid in the blood. Excess fluid, primarily salt and water, may build up throughout the body resulting in weight gain.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### Liquid Pharmaceutical Formulations of Loop Diuretics

As described herein, in one aspect, the invention provides liquid pharmaceutical formulations of a loop diuretic, for example, a loop diuretic, and a pharmaceutically acceptable excipient.

In various embodiments, a liquid pharmaceutical formulation generally comprises:
(i) a loop diuretic, or a pharmaceutically acceptable salt thereof, in a therapeutically effective amount; and
(ii) a pharmaceutically acceptable buffer.

### Loop Diuretics

In certain embodiments, the concentration of the loop diuretic, or a pharmaceutically acceptable salt thereof, in a liquid pharmaceutical formulation described herein can be from about 0.025 mg/mL to about 250 mg/mL, from about 0.1 mg/mL to about 250 mg/mL, from about 1 mg/mL to about 250 mg/mL, from about 5 mg/mL to about 250 mg/mL, from about 10 mg/mL to about 250 mg/mL, from about 20 mg/mL to about 250 mg/mL, from about 30 mg/mL to about 250 mg/mL, from about 40 mg/mL to about 250 mg/mL, from about 50 mg/mL to about 250 mg/mL, from about 60 mg/mL to about 250 mg/mL, from about 70 mg/mL to about 250 mg/mL, from about 80 mg/mL to about 250 mg/mL, from about 90 mg/mL to about 250 mg/mL, from about 100 mg/mL to about 250 mg/mL, from about 120 mg/mL to about 250 mg/mL, from about 140 mg/mL to about 250 mg/mL, from about 160 mg/mL to about 250 mg/mL, from about 180 mg/mL to about 250 mg/mL, from about 200 mg/mL to about 250 mg/mL, from about 225 mg/mL to about 250 mg/mL, from about 0.025 mg/mL to about 225 mg/mL, from about 0.025 mg/mL to about 200 mg/mL, from about 0.025 mg/mL to about 180 mg/mL, from about 0.025 mg/mL to about 160 mg/mL, from about 0.025 mg/mL to about 140 mg/mL, from about 0.025 mg/mL to about 120 mg/mL, from about 0.025 mg/mL to about 100 mg/mL, from about 0.025 mg/mL to about 90 mg/mL, from about 0.025 mg/mL to about 80 mg/mL, from about 0.025 mg/mL to about 70 mg/mL, from about 0.025 mg/mL to about 60 mg/mL, from about 0.025 mg/mL to about 50 mg/mL, from about 0.025 mg/mL to about 40 mg/mL, from about 0.025 mg/mL to about 30 mg/mL, from about 0.025 mg/mL to about 20 mg/mL, from about 0.025 mg/mL to about 10 mg/mL, from about 0.025 mg/mL to about 5 mg/mL, from about 0.025 mg/mL to about 1 mg/mL, from about 0.025 mg/mL to about 0.1 mg/mL, from about 0.1 mg/mL to about 225 mg/mL, from about 0.1 mg/mL to about 200 mg/mL, from about 0.1 mg/mL to about 180 mg/mL, from about 0.1 mg/mL to about 160 mg/mL, from about 0.1 mg/mL to about 140 mg/mL, from about 0.1 mg/mL to about 120 mg/mL, from about 0.1 mg/mL to about 100 mg/mL, from about 0.1 mg/mL to about 90 mg/mL, from about 0.1 mg/mL to about 80 mg/mL, from about 0.1 mg/mL to about 70 mg/mL, from about 0.1 mg/mL to about 60 mg/mL, from about 0.1 mg/mL to about 50 mg/mL, from about 0.1 mg/mL to about 40 mg/mL, from about 0.1 mg/mL to about 30 mg/mL, from about 0.1 mg/mL to about 20 mg/mL, from about 0.1 mg/ml to about 10 mg/mL, from about 0.1 mg/mL to about 5 mg/mL, from about 0.1 mg/mL to about 1 mg/mL, from about 1 mg/mL to about 225 mg/mL, from about 1 mg/mL to about 200 mg/mL, from about 1 mg/mL to about 180 mg/mL, from about 1 mg/mL to about 160 mg/mL, from about 1 mg/mL to about 140 mg/mL, from about 1 mg/mL to about 120 mg/mL, from about 1 mg/mL to about 100 mg/mL, from about 1 mg/mL to about 90 mg/mL, from about 1 mg/mL to about 80 mg/mL, from about 1 mg/mL to about 70 mg/mL, from about 1 mg/mL to about 60 mg/mL, from about 1 mg/mL to about 50 mg/mL, from about 1 mg/mL to about 40 mg/mL, from about 1 mg/mL to about 30 mg/mL, from about 1 mg/mL to about 20 mg/mL, from about 1 mg/mL to about 10 mg/mL, from about 1 mg/mL to about 5 mg/mL, from about 5 mg/mL to about 225 mg/mL, from about 5 mg/mL to about 200 mg/mL, from about 5 mg/mL to about 180 mg/mL, from about 5 mg/mL to about 160 mg/mL, from about 5 mg/mL to about 140 mg/mL, from about 5 mg/mL to about 120 mg/mL, from about 5 mg/mL to about 100 mg/mL, from about 5 mg/mL to about 90 mg/mL, from about 5 mg/mL to about 80 mg/mL, from about 5 mg/mL to about 70 mg/mL, from about 5 mg/mL to about 60 mg/mL, from about 5 mg/mL to about 50 mg/mL, from about 5 mg/mL to about 40 mg/mL, from about 5 mg/mL to about 30 mg/mL, from about 5 mg/mL to about 20 mg/mL, from about 5 mg/mL to about 10 mg/mL, from about 10 mg/mL to about 225 mg/mL, from about 10 mg/mL to about 200 mg/mL, from about 10 mg/mL to about 180 mg/mL, from about 10 mg/mL to about 160 mg/mL, from about 10 mg/mL to about 140 mg/mL, from about 10 mg/mL to about 120 mg/mL, from about 10 mg/mL to about 100 mg/mL, from about 10 mg/mL to about 90 mg/mL, from about 10 mg/mL to about 80 mg/mL, from about 10 mg/mL to about 70 mg/mL, from about 10 mg/mL to about 60 mg/mL, from about 10 mg/mL to about 50 mg/mL, from about 10 mg/mL to about 40 mg/mL, from about 10 mg/mL to about 30 mg/mL, from about 10 mg/mL to about 20 mg/mL, from about 20 mg/mL to about 225 mg/mL, from about 20 mg/mL to about 200 mg/mL, from about 20 mg/mL to about 180 mg/mL, from about 20 mg/mL to about 160 mg/mL, from about 20 mg/mL to about 140 mg/mL, from about 20 mg/mL to about 120 mg/mL, from about 20 mg/mL to about 100 mg/mL, from about 20 mg/mL to about 90 mg/mL, from about 20 mg/mL to about 80 mg/mL, from about 20 mg/mL to about 70 mg/mL, from about 20 mg/mL to about 60 mg/mL, from about 20 mg/mL to about 50 mg/mL, from about 20 mg/mL to about 40 mg/mL, from about 20 mg/mL to about 30 mg/mL, from about 30 mg/mL to about 225 mg/mL, from about 30 mg/mL to about 200 mg/mL, from about 30 mg/mL to about 180 mg/mL, from about 30 mg/mL to about 160 mg/mL, from about 30 mg/mL to about 140 mg/mL, from about 30 mg/mL to about 120 mg/mL, from about 30 mg/mL to about 100 mg/mL, from about 30 mg/mL to about 90 mg/mL, from about 30 mg/mL to about 80 mg/mL, from about 30 mg/mL to about 70 mg/mL, from about 30 mg/mL to about 60 mg/mL, from about 30 mg/mL to about 50 mg/mL, from about 30 mg/mL to about 40 mg/mL, from about 40 mg/mL to about 225 mg/mL, from about 40 mg/mL to about 200 mg/mL, from about 40 mg/mL to about 180 mg/mL, from about 40 mg/mL to about 160 mg/mL, from about 40 mg/mL to about 140 mg/mL, from about 40 mg/mL to about 120 mg/mL, from about 40 mg/mL to about 100 mg/mL, from about 40 mg/mL to about 90 mg/mL, from about 40 mg/mL to about 80 mg/mL, from about 40 mg/mL to about 70 mg/mL, from about 40 mg/mL to about 60 mg/mL, from about 40 mg/mL to about 50 mg/mL, from about 50 mg/mL to about 225 mg/mL, from about 50 mg/mL to about 200 mg/mL, from about 50 mg/mL to about 180 mg/mL, from about 50 mg/mL to about 160 mg/mL, from about 50 mg/mL to about 140 mg/mL, from about 50 mg/mL to about 120 mg/mL, from about 50 mg/mL to about 100 mg/mL, from about 50 mg/mL to about 90 mg/mL, from about 50 mg/mL to about 80 mg/mL, from about 50 mg/mL to about 70 mg/mL, from about 50 mg/mL to about 60 mg/mL, from about 60 mg/mL to about 225 mg/mL, from about 60 mg/mL to about 200 mg/mL, from about 60 mg/mL to about 180 mg/mL, from about 60 mg/mL to about 160 mg/mL, from about 60 mg/mL to about 140 mg/mL, from about 60 mg/mL to about 120 mg/mL, from about 60 mg/mL to about 100 mg/mL, from about 60 mg/mL to about 90 mg/mL, from about 60 mg/mL to about 80 mg/mL, from about 60 mg/mL to about 70 mg/mL, from about 70 mg/mL to about 225 mg/mL, from about 70 mg/mL to about 200 mg/mL, from about 70 mg/mL to about 180 mg/mL, from about 70 mg/mL to about 160 mg/mL, from about 70 mg/mL to about 140 mg/mL, from about 70 mg/mL to about 120 mg/mL, from about 70 mg/mL to about 100 mg/mL, from about 70 mg/mL to about 90 mg/mL, from about 70 mg/mL to about 80 mg/mL, from about 70 mg/mL to about 225 mg/mL, from about 80 mg/mL to about 200 mg/mL, from about 80 mg/mL to about 180 mg/mL, from about 80 mg/mL to about 160 mg/mL, from about 80 mg/mL to about 140 mg/mL, from about 80 mg/mL to about 120 mg/mL, from about 80 mg/mL to about 100 mg/mL, from about 80 mg/mL to about 90 mg/mL, from about 70 mg/mL to about 225 mg/mL, from about 90 mg/mL to about 200 mg/mL, from about 90 mg/mL to about 180 mg/mL, from about 90 mg/mL to about 160 mg/mL, from about 90 mg/mL to about 140 mg/mL, from about 90 mg/mL to about 120 mg/mL, from about 90 mg/mL to about 100 mg/mL, from about 100 mg/mL to about 225 mg/mL, from about 100 mg/mL to about 200 mg/mL, from about 100 mg/mL to about 180 mg/mL, from about 100 mg/mL to about 160 mg/mL, from about 100 mg/mL to about 140 mg/mL, from about 100 mg/mL to about 120 mg/mL, from about 120 mg/mL to about 225 mg/mL, from about 120 mg/mL to about 200 mg/mL, from about 120 mg/mL to about 180 mg/mL, from about 120 mg/mL to about 160 mg/mL, from about 120 mg/mL to about 140 mg/mL, from about 140 mg/mL to about 225 mg/mL, from about 140 mg/ml to about 200 mg/mL, from about 140 mg/mL to about 180 mg/mL, from about 140 mg/mL to about 160 mg/mL, from about 160 mg/mL to about 225 mg/mL, from about 160 mg/mL to about 200 mg/mL, from about 160 mg/mL to about 180 mg/mL, from about 180 mg/mL to about 225 mg/mL, from about 180 mg/mL to about 200 mg/mL, or from about 200 mg/mL to about 225 mg/mL. In certain embodiments, the concentration of the loop diuretic, or a pharmaceutically acceptable salt thereof, in a liquid pharmaceutical formulation described herein can be from about 0.025 mg/mL to about 250 mg/mL.

In certain embodiments, the concentration of the loop diuretic, or a pharmaceutically acceptable salt thereof, in the liquid pharmaceutical formulations described herein can be greater than about 0.025 mg/mL, greater than about 0.1 mg/mL, greater than about 1 mg/mL, greater than about 5 mg/mL, greater than about 10 mg/mL, greater than about 20 mg/mL, greater than about 30 mg/mL, greater than about 40 mg/mL, greater than about 50 mg/mL, greater than about 60 mg/mL, greater than about 70 mg/mL, greater than about 80 mg/mL, greater than about 90 mg/mL, greater than about 100 mg/mL, greater than about 110 mg/mL, greater than about 120 mg/mL, greater than about 130 mg/mL, greater than about 140 mg/mL, greater than about 150 mg/mL, greater than about 160 mg/mL, greater than about 170 mg/mL, greater than about 180 mg/mL, greater than about 190 mg/mL, greater than about 200 mg/mL, greater than about 210 mg/mL, greater than about 220 mg/mL, greater than about 230 mg/mL, greater than about 240 mg/mL, or greater than about 250 mg/mL.

In certain embodiments, the concentration of the loop diuretic, or a pharmaceutically acceptable salt thereof, in the liquid pharmaceutical formulations described herein can be about 0.025 mg/mL, about 0.05 mg/mL, about 0.075 mg/mL, about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 22 mg/mL, about 24 mg/mL, about 26 mg/mL, about 28 mg/mL, about 30 mg/mL, about 32 mg/mL, about 34 mg/mL, about 36 mg/mL, about 38 mg/mL, about 40 mg/mL, about 42 mg/mL, about 44 mg/mL, about 46 mg/mL, about 48 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, about 200 mg/mL, about 210 mg/mL, about 220 mg/mL, about 230 mg/mL, about 240 mg/mL, or about 250 mg/mL.

In certain embodiments, the loop diuretic is selected from the group consisting of furosemide, bumetanide, ethacrynic acid, torsemide, or a pharmaceutically acceptable salt thereof. In certain embodiments, the loop diuretic is furosemide, or a pharmaceutically acceptable salt thereof. In certain embodiments, the loop diuretic is bumetanide, or a pharmaceutically acceptable salt thereof. In certain embodiments, the loop diuretic is ethacrynic acid, or a pharmaceutically acceptable salt thereof. In certain embodiments, the loop diuretic is torsemide, or a pharmaceutically acceptable salt thereof.

In certain embodiments, the concentration of furosemide in a liquid pharmaceutical formulation described herein can be from about 1 mg/mL to about 250 mg/mL, from about 5 mg/mL to about 250 mg/mL, from about 10 mg/mL to about 250 mg/mL, from about 20 mg/mL to about 250 mg/mL, from about 30 mg/mL to about 250 mg/mL, from about 40 mg/mL to about 250 mg/mL, from about 50 mg/mL to about 250 mg/mL, from about 60 mg/mL to about 250 mg/mL, from about 70 mg/mL to about 250 mg/mL, from about 80 mg/mL to about 250 mg/mL, from about 90 mg/mL to about 250 mg/mL, from about 100 mg/mL to about 250 mg/mL, from about 120 mg/mL to about 250 mg/mL, from about 140 mg/mL to about 250 mg/mL, from about 160 mg/mL to about 250 mg/mL, from about 180 mg/mL to about 250 mg/mL, from about 200 mg/mL to about 250 mg/mL, from about 225 mg/mL to about 250 mg/mL, from about 1 mg/ml to about 225 mg/mL, from about 1 mg/mL to about 200 mg/mL, from about 1 mg/mL to about 180 mg/mL, from about 1 mg/mL to about 160 mg/mL, from about 1 mg/mL to about 140 mg/mL, from about 1 mg/mL to about 120 mg/mL, from about 1 mg/mL to about 100 mg/mL, from about 1 mg/mL to about 90 mg/mL, from about 1 mg/mL to about 80 mg/mL, from about 1 mg/mL to about 70 mg/mL, from about 1 mg/mL to about 60 mg/mL, from about 1 mg/mL to about 50 mg/mL, from about 1 mg/mL to about 40 mg/mL, from about 1 mg/mL to about 30 mg/mL, from about 1 mg/mL to about 20 mg/mL, from about 1 mg/mL to about 10 mg/mL, from about 1 mg/mL to about 5 mg/mL, from about 5 mg/mL to about 225 mg/mL, from about 5 mg/mL to about 200 mg/mL, from about 5 mg/mL to about 180 mg/mL, from about 5 mg/mL to about 160 mg/mL, from about 5 mg/mL to about 140 mg/mL, from about 5 mg/mL to about 120 mg/mL, from about 5 mg/mL to about 100 mg/mL, from about 5 mg/mL to about 90 mg/mL, from about 5 mg/mL to about 80 mg/mL, from about 5 mg/mL to about 70 mg/mL, from about 5 mg/mL to about 60 mg/mL, from about 5 mg/mL to about 50 mg/mL, from about 5 mg/mL to about 40 mg/mL, from about 5 mg/mL to about 30 mg/mL, from about 5 mg/mL to about 20 mg/mL, from about 5 mg/mL to about 10 mg/mL, from about 10 mg/mL to about 225 mg/mL, from about 10 mg/mL to about 200 mg/mL, from about 10 mg/mL to about 180 mg/mL, from about 10 mg/mL to about 160 mg/mL, from about 10 mg/mL to about 140 mg/mL, from about 10 mg/mL to about 120 mg/mL, from about 10 mg/mL to about 100 mg/mL, from about 10 mg/mL to about 90 mg/mL, from about 10 mg/mL to about 80 mg/mL, from about 10 mg/mL to about 70 mg/mL, from about 10 mg/mL to about 60 mg/mL, from about 10 mg/mL to about 50 mg/mL, from about 10 mg/mL to about 40 mg/mL, from about 10 mg/mL to about 30 mg/mL, from about 10 mg/mL to about 20 mg/mL, from about 20 mg/mL to about 225 mg/mL, from about 20 mg/mL to about 200 mg/mL, from about 20 mg/mL to about 180 mg/mL, from about 20 mg/mL to about 160 mg/mL, from about 20 mg/mL to about 140 mg/mL, from about 20 mg/mL to about 120 mg/mL, from about 20 mg/mL to about 100 mg/mL, from about 20 mg/mL to about 90 mg/mL, from about 20 mg/mL to about 80 mg/mL, from about 20 mg/mL to about 70 mg/mL, from about 20 mg/mL to about 60 mg/mL, from about 20 mg/mL to about 50 mg/mL, from about 20 mg/mL to about 40 mg/mL, from about 20 mg/mL to about 30 mg/mL, from about 30 mg/mL to about 225 mg/mL, from about 30 mg/mL to about 200 mg/mL, from about 30 mg/mL to about 180 mg/mL, from about 30 mg/mL to about 160 mg/mL, from about 30 mg/mL to about 140 mg/mL, from about 30 mg/mL to about 120 mg/mL, from about 30 mg/mL to about 100 mg/mL, from about 30 mg/mL to about 90 mg/mL, from about 30 mg/mL to about 80 mg/mL, from about 30 mg/mL to about 70 mg/mL, from about 30 mg/mL to about 60 mg/mL, from about 30 mg/mL to about 50 mg/mL, from about 30 mg/mL to about 40 mg/mL, from about 40 mg/mL to about 225 mg/mL, from about 40 mg/mL to about 200 mg/mL, from about 40 mg/mL to about 180 mg/mL, from about 40 mg/mL to about 160 mg/mL, from about 40 mg/mL to about 140 mg/mL, from about 40 mg/mL to about 120 mg/mL, from about 40 mg/mL to about 100 mg/mL, from about 40 mg/mL to about 90 mg/mL, from about 40 mg/mL to about 80 mg/mL, from about 40 mg/mL to about 70 mg/mL, from about 40 mg/mL to about 60 mg/mL, from about 40 mg/mL to about 50 mg/mL, from about 50 mg/mL to about 225 mg/mL, from about 50 mg/mL to about 200 mg/mL, from about 50 mg/mL to about 180 mg/mL, from about 50 mg/mL to about 160 mg/mL, from about 50 mg/mL to about 140 mg/mL, from about 50 mg/mL to about 120 mg/mL, from about 50 mg/mL to about 100 mg/mL, from about 50 mg/mL to about 90 mg/mL, from about 50 mg/mL to about 80 mg/mL, from about 50 mg/mL to about 70 mg/mL, from about 50 mg/mL to about 60 mg/mL, from about 60 mg/mL to about 225 mg/mL, from about 60 mg/mL to about 200 mg/mL, from about 60 mg/mL to about 180 mg/mL, from about 60 mg/mL to about 160 mg/mL, from about 60 mg/mL to about 140 mg/mL, from about 60 mg/mL to about 120 mg/mL, from about 60 mg/mL to about 100 mg/mL, from about 60 mg/mL to about 90 mg/mL, from about 60 mg/mL to about 80 mg/mL, from about 60 mg/mL to about 70 mg/mL, from about 70 mg/mL to about 225 mg/mL, from about 70 mg/mL to about 200 mg/mL, from about 70 mg/mL to about 180 mg/mL, from about 70 mg/mL to about 160 mg/mL, from about 70 mg/mL to about 140 mg/mL, from about 70 mg/mL to about 120 mg/mL, from about 70 mg/mL to about 100 mg/mL, from about 70 mg/mL to about 90 mg/mL, from about 70 mg/mL to about 80 mg/mL, from about 70 mg/mL to about 225 mg/mL, from about 80 mg/mL to about 200 mg/mL, from about 80 mg/mL to about 180 mg/mL, from about 80 mg/mL to about 160 mg/mL, from about 80 mg/mL to about 140 mg/mL, from about 80 mg/mL to about 120 mg/mL, from about 80 mg/mL to about 100 mg/mL, from about 80 mg/mL to about 90 mg/mL, from about 70 mg/mL to about 225 mg/mL, from about 90 mg/mL to about 200 mg/mL, from about 90 mg/mL to about 180 mg/mL, from about 90 mg/mL to about 160 mg/mL, from about 90 mg/mL to about 140 mg/mL, from about 90 mg/mL to about 120 mg/mL, from about 90 mg/mL to about 100 mg/mL, from about 100 mg/mL to about 225 mg/mL, from about 100 mg/mL to about 200 mg/mL, from about 100 mg/mL to about 180 mg/mL, from about 100 mg/mL to about 160 mg/mL, from about 100 mg/mL to about 140 mg/mL, from about 100 mg/mL to about 120 mg/mL, from about 120 mg/mL to about 225 mg/mL, from about 120 mg/mL to about 200 mg/mL, from about 120 mg/mL to about 180 mg/mL, from about 120 mg/mL to about 160 mg/mL, from about 120 mg/mL to about 140 mg/mL, from about 140 mg/mL to about 225 mg/mL, from about 140 mg/mL to about 200 mg/mL, from about 140 mg/mL to about 180 mg/mL, from about 140 mg/mL to about 160 mg/mL, from about 160 mg/mL to about 225 mg/mL, from about 160 mg/mL to about 200 mg/mL, from about 160 mg/mL to about 180 mg/mL, from about 180 mg/mL to about 225 mg/mL, from about 180 mg/mL to about 200 mg/mL, or from about 200 mg/mL to about 225 mg/mL. In certain embodiments, the concentration of furosemide in a liquid pharmaceutical formulation described herein can be from about 1 mg/mL to about 250 mg/mL.

In certain embodiments, the concentration of furosemide in the liquid pharmaceutical formulations described herein can be greater than about 1 mg/mL, greater than about 5 mg/mL, greater than about 10 mg/mL, greater than about 20 mg/mL, greater than about 30 mg/mL, greater than about 40 mg/mL, greater than about 50 mg/mL, greater than about 60 mg/mL, greater than about 70 mg/mL, greater than about 80 mg/mL, greater than about 90 mg/mL, greater than about 100 mg/mL, greater than about 110 mg/mL, greater than about 120 mg/mL, greater than about 130 mg/mL, greater than about 140 mg/mL, greater than about 150 mg/mL, greater than about 160 mg/mL, greater than about 170 mg/mL, greater than about 180 mg/mL, greater than about 190 mg/mL, greater than about 200 mg/mL, greater than about 210 mg/mL, greater than about 220 mg/mL, greater than about 230 mg/mL, greater than about 240 mg/mL, or greater than about 250 mg/mL.

In certain embodiments, the concentration of furosemide in the liquid pharmaceutical formulations described herein can be about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 22 mg/mL, about 24 mg/mL, about 26 mg/mL, about 28 mg/mL, about 30 mg/mL, about 32 mg/mL, about 34 mg/mL, about 36 mg/mL, about 38 mg/mL, about 40 mg/mL, about 42 mg/mL, about 44 mg/mL, about 46 mg/mL, about 48 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, about 200 mg/mL, about 210 mg/mL, about 220 mg/mL, about 230 mg/mL, about 240 mg/mL, or about 250 mg/mL. In certain embodiments, the concentration of furosemide in the liquid pharmaceutical formulations described herein can be about 80 mg/mL.

In certain embodiments, the concentration of bumetanide in a liquid pharmaceutical formulation described herein can be from about 0.025 mg/mL to about 65 mg/mL, from about 0.1 mg/mL to about 65 mg/mL, from about 0.5 mg/mL to about 65 mg/mL, from about 1 mg/ml to about 65 mg/mL, from about 5 mg/mL to about 65 mg/mL, from about 10 mg/mL to about 65 mg/mL, from about 20 mg/mL to about 65 mg/mL, from about 30 mg/mL to about 65 mg/mL, from about 40 mg/mL to about 65 mg/mL, from about 50 mg/mL to about 65 mg/mL, from about 60 mg/mL to about 65 mg/mL, from about 0.025 mg/mL to about 60 mg/mL, from about 0.025 mg/mL to about 50 mg/mL, from about 0.025 mg/mL to about 40 mg/mL, from about 0.025 mg/mL to about 30 mg/mL, from about 0.025 mg/mL to about 20 mg/mL, from about 0.025 mg/mL to about 10 mg/mL, from about 0.025 mg/mL to about 5 mg/mL, from about 0.025 mg/mL to about 1 mg/mL, from about 0.025 mg/mL to about 0.5 mg/mL, from about 0.025 mg/mL to about 0.1 mg/mL, from about 0.1 mg/mL to about 60 mg/mL, from about 0.1 mg/mL to about 50 mg/mL, from about 0.1 mg/mL to about 40 mg/mL, from about 0.1 mg/mL to about 30 mg/mL, from about 0.1 mg/mL to about 20 mg/mL, from about 0.1 mg/mL to about 10 mg/mL, from about 0.1 mg/mL to about 5 mg/mL, from about 0.1 mg/mL to about 1 mg/mL, from about 0.5 mg/mL to about 60 mg/mL, from about 0.5 mg/mL to about 50 mg/mL, from about 0.5 mg/mL to about 40 mg/mL, from about 0.5 mg/mL to about 30 mg/mL, from about 0.5 mg/mL to about 20 mg/mL, from about 0.5 mg/mL to about 10 mg/mL, from about 0.5 mg/mL to about 10 mg/mL, from about 0.5 mg/mL to about 5 mg/mL, from about 0.5 mg/mL to about 1 mg/mL, from about 1 mg/mL to about 60 mg/mL, from about 1 mg/mL to about 50 mg/mL, from about 1 mg/mL to about 40 mg/mL, from about 1 mg/mL to about 30 mg/mL, from about 1 mg/mL to about 20 mg/mL, from about 1 mg/mL to about 10 mg/mL, from about 1 mg/mL to about 5 mg/mL, from about 5 mg/mL to about 60 mg/mL, from about 5 mg/mL to about 50 mg/mL, from about 5 mg/mL to about 40 mg/mL, from about 5 mg/mL to about 30 mg/mL, from about 5 mg/mL to about 20 mg/mL, from about 5 mg/mL to about 10 mg/mL, from about 10 mg/mL to about 60 mg/mL, from about 10 mg/mL to about 50 mg/mL, from about 10 mg/mL to about 40 mg/mL, from about 10 mg/mL to about 30 mg/mL, from about 10 mg/mL to about 20 mg/mL, from about 20 mg/mL to about 60 mg/mL, from about 20 mg/mL to about 50 mg/mL, from about 20 mg/mL to about 40 mg/mL, from about 20 mg/mL to about 30 mg/mL, from about 30 mg/mL to about 60 mg/mL, from about 30 mg/mL to about 50 mg/mL, from about 30 mg/mL to about 40 mg/mL, from about 40 mg/mL to about 60 mg/mL, from about 40 mg/mL to about 50 mg/mL, or from about 50 mg/mL to about 60 mg/mL. In certain embodiments, the concentration of bumetanide in a liquid pharmaceutical formulation described herein can be from about 0.025 mg/mL to about 65 mg/mL.

In certain embodiments, the concentration of bumetanide in the liquid pharmaceutical formulations described herein can be greater than about 0.025 mg/mL, greater than about 1 mg/mL, greater than about 5 mg/mL, greater than about 10 mg/mL, greater than about 20 mg/mL, greater than about 30 mg/mL, greater than about 40 mg/mL, greater than about 50 mg/mL, or greater than about 60 mg/mL.

In certain embodiments, the concentration of bumetanide in the liquid pharmaceutical formulations described herein can be about 0.025 mg/mL, about 0.05 mg/mL, about 0.075 mg/mL, about 0.1 mg/mL, about 0.25 mg/mL, about 0.5 mg/mL, about 0.75 mg/mL, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 22 mg/mL, about 24 mg/mL, about 26 mg/mL, about 28 mg/mL, about 30 mg/mL, about 32 mg/mL, about 34 mg/mL, about 36 mg/mL, about 38 mg/mL, about 40 mg/mL, about 42 mg/mL, about 44 mg/mL, about 46 mg/mL, about 48 mg/mL, about 50 mg/mL, about 55 mg/mL, about 60 mg/mL, or about 65 mg/mL.

In certain embodiments, the concentration of torsemide in a liquid pharmaceutical formulation described herein can be from about 0.5 mg/mL to about 125 mg/mL, from about 1 mg/mL to about 125 mg/mL, from about 5 mg/mL to about 125 mg/mL, from about 10 mg/mL to about 125 mg/mL, from about 20 mg/mL to about 125 mg/mL, from about 30 mg/mL to about 125 mg/mL, from about 40 mg/mL to about 125 mg/mL, from about 50 mg/mL to about 125 mg/mL, from about 75 mg/mL to about 125 mg/mL, from about 100 mg/mL to about 125 mg/mL, from about 0.5 mg/mL to about 100 mg/mL, from about 0.5 mg/mL to about 75 mg/mL, from about 0.5 mg/mL to about 50 mg/mL, from about 0.5 mg/mL to about 40 mg/mL, from about 0.5 mg/mL to about 30 mg/mL, from about 0.5 mg/mL to about 20 mg/mL, from about 0.5 mg/mL to about 10 mg/mL, from about 0.5 mg/mL to about 5 mg/mL, from about 0.5 mg/mL to about 1 mg/mL, from about 1 mg/mL to about 100 mg/mL, from about 1 mg/mL to about 75 mg/mL, from about 1 mg/mL to about 50 mg/mL, from about 1 mg/mL to about 40 mg/mL, from about 1 mg/mL to about 30 mg/mL, from about 1 mg/mL to about 20 mg/mL, from about 1 mg/mL to about 10 mg/mL, from about 1 mg/mL to about 5 mg/mL, from about 5 mg/mL to about 100 mg/mL, from about 5 mg/mL to about 75 mg/mL, from about 5 mg/mL to about 50 mg/mL, from about 5 mg/mL to about 40 mg/mL, from about 5 mg/mL to about 30 mg/mL, from about 5 mg/mL to about 20 mg/mL, from about 5 mg/mL to about 10 mg/mL, from about 10 mg/mL to about 100 mg/mL, from about 10 mg/mL to about 75 mg/mL, from about 10 mg/mL to about 50 mg/mL, from about 10 mg/mL to about 40 mg/mL, from about 10 mg/mL to about 30 mg/mL, from about 10 mg/mL to about 20 mg/mL, from about 20 mg/mL to about 100 mg/mL, from about 20 mg/mL to about 75 mg/mL, from about 20 mg/mL to about 50 mg/mL, from about 20 mg/mL to about 40 mg/mL, from about 20 mg/mL to about 30 mg/mL, from about 30 mg/mL to about 100 mg/mL, from about 30 mg/mL to about 75 mg/mL, from about 30 mg/mL to about 50 mg/mL, from about 30 mg/mL to about 40 mg/mL, from about 40 mg/mL to about 100 mg/mL, from about 40 mg/mL to about 75 mg/mL, from about 40 mg/mL to about 50 mg/mL, from about 50 mg/mL to about 100 mg/mL, from about 50 mg/mL to about 75 mg/mL, or from about 75 mg/mL to about 100 mg/mL.

In certain embodiments, the concentration of torsemide in the liquid pharmaceutical formulations described herein can be greater than about 0.5 mg/mL, greater than about 1 mg/ml, greater than about 5 mg/mL, greater than about 10 mg/mL, greater than about 20 mg/mL, greater than about 30 mg/mL, greater than about 40 mg/mL, greater than about 50 mg/mL, greater than about 60 mg/mL, greater than about 70 mg/mL, greater than about 80 mg/mL, greater than about 90 mg/mL, greater than about 100 mg/mL, or greater than about 125 mg/mL.

In certain embodiments, the concentration of torsemide in the liquid pharmaceutical formulations described herein can be about 0.5 mg/mL, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL, about 100 mg/mL, or about 125 mg/mL.

### pH, Buffering Agents & pH Modifiers

In certain embodiments, the pH of a liquid pharmaceutical formulation described herein can be from about 5.5 to about 8.5, from about 6 to about 8.5, from about 6.5 to about 8.5, from about 7 to about 8.5, from about 7.5 to about 8.5, from about 8 to about 8.5, from about 5.5 to about 8, from about 5.5 to about 7.5, from about 5.5 to about 7, from about 5.5 to about 6.5, from about 5.5 to about 6, from about 6 to about 8, from about 6 to about 7.5, from about 6 to about 7, from about 6 to about 6.5, from about 6.5 to about 8, from about 6.5 to about 7.5, from about 6.5 to about 7, from about 7 to about 8, from about 7 to about 7.5, or from about 7.5 to about 8. In certain embodiments, the pH of a liquid pharmaceutical formulation described herein is from about 6.5 to about 8.5.

In certain embodiments, the pH of a liquid pharmaceutical formulation described herein can be from about 7 to about 8.5, from about 7.1 to about 8.5, from about 7.3 to about 8.5, from about 7.5 to about 8.5, from about 7.7 to about 8.5, from about 7.9 to about 8.5, from about 8.1 to about 8.5, from about 8.3 to about 8.5, about 7 to about 8.3, about 7 to about 8.1, about 7 to about 7.9, about 7 to about 7.7, about 7 to about 7.5, about 7 to about 7.3, about 7 to about 7.1, about 7.1 to about 8.3, about 7.1 to about 8.1, about 7.1 to about 7.9, about 7.1 to about 7.7, about 7.1 to about 7.5, about 7.1 to about 7.3, about 7.3 to about 8.3, about 7.3 to about 8.1, about 7.3 to about 7.9, about 7.3 to about 7.7, about 7.3 to about 7.5, about 7.5 to about 8.3, about 7.5 to about 8.1, about 7.5 to about 7.9, about 7.5 to about 7.7, about 7.7 to about 8.3, about 7.7 to about 8.1, about 7.7 to about 7.9, about 7.9 to about 8.3, about 7.9 to about 8.1, or about 8.1 to about 8.3.

In certain embodiments, the pH of a liquid pharmaceutical formulation described herein can be about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, about 8, about 8.1, about 8.2, about 8.3, about 8.4, or about 8.5. In certain embodiments, the pH of a liquid pharmaceutical formulation described herein can be about 7.4. In certain embodiments, the pH of a liquid pharmaceutical formulation described herein can be about 7.7.

In certain embodiments, the pH of a liquid pharmaceutical formulation described herein can be 5.5±0.3, 5.6±0.3, 5.7±0.3, 5.8±0.3, 5.9±0.3, 6±0.3, 6.1±0.3, 6.2±0.3, 6.3±0.3, 6.4±0.3, 6.5±0.3, 6.6±0.3, 6.7±0.3, 6.8±0.3, 6.9±0.3, 7±0.3, 7.1±0.3, 7.2±0.3, 7.3±0.3, 7.4±0.3, 7.5±0.3, 7.6±0.3, 7.7±0.3, 7.8±0.3, 7.9±0.3, 8±0.3, 8.1±0.3, 8.2±0.3, 8.3±0.3, 8.4±0.3, or 8.5±0.3. In certain embodiments, the pH of a liquid pharmaceutical formulation described herein can be 7.4±0.3. In certain embodiments, the pH of a liquid pharmaceutical formulation described herein can be 7.7±0.3.

### (1) Pharmaceutically Acceptable Buffers

In certain embodiments, the concentration of the pharmaceutically acceptable buffer in a liquid pharmaceutical formulation described herein can be from about 10 mM to about 500 mM, from about 20 mM to about 500 mM, from about 30 mM to about 500 mM, from about 40 mM to about 500 mM, from about 50 mM to about 500 mM, from about 60 mM to about 500 mM, from about 70 mM to about 500 mM, from about 80 mM to about 500 mM, from about 90 mM to about 500 mM, from about 100 mM to about 500 mM, from about 125 mM to about 500 mM, from about 150 mM to about 500 mM, from about 200 mM to about 500 mM, from about 250 mM to about 500 mM, from about 300 mM to about 500 mM, from about 350 mM to about 500 mM, from about 400 mM to about 500 mM, from about 450 mM to about 500 mM, from about 10 mM to about 450 mM, from about 10 mM to about 400 mM, from about 10 mM to about 350 mM, from about 10 mM to about 300 mM, from about 10 mM to about 250 mM, from about 10 mM to about 200 mM, from about 10 mM to about 150 mM, from about 10 mM to about 125 mM, from about 10 mM to about 100 mM, from about 10 mM to about 90 mM, from about 10 mM to about 80 mM, from about 10 mM to about 70 mM, from about 10 mM to about 60 mM, from about 10 mM to about 50 mM, from about 10 mM to about 40 mM, from about 10 mM to about 30 mM, from about 10 mM to about 20 mM, from about 20 mM to about 450 mM, from about 20 mM to about 400 mM, from about 20 mM to about 350 mM, from about 20 mM to about 300 mM, from about 20 mM to about 250 mM, from about 20 mM to about 200 mM, from about 20 mM to about 150 mM, from about 20 mM to about 100 mM, from about 20 mM to about 90 mM, from about 20 mM to about 80 mM, from about 20 mM to about 70 mM, from about 20 mM to about 60 mM, from about 20 mM to about 50 mM, from about 20 mM to about 40 mM, from about 20 mM to about 30 mM, from about 30 mM to about 450 mM, from about 30 mM to about 400 mM, from about 30 mM to about 350 mM, from about 30 mM to about 300 mM, from about 30 mM to about 250 mM, from about 30 mM to about 200 mM, from about 30 mM to about 150 mM, from about 30 mM to about 125 mM, from about 30 mM to about 100 mM, from about 30 mM to about 90 mM, from about 30 mM to about 80 mM, from about 30 mM to about 70 mM, from about 30 mM to about 60 mM, from about 30 mM to about 50 mM, from about 30 mM to about 40 mM, from about 40 mM to about 450 mM, from about 40 mM to about 400 mM, from about 40 mM to about 350 mM, from about 40 mM to about 300 mM, from about 40 mM to about 250 mM, from about 40 mM to about 200 mM, from about 40 mM to about 150 mM, from about 40 mM to about 125 mM, from about 40 mM to about 100 mM, from about 40 mM to about 90 mM, from about 40 mM to about 80 mM, from about 40 mM to about 70 mM, from about 40 mM to about 60 mM, from about 40 mM to about 50 mM, from about 50 mM to about 450 mM, from about 50 mM to about 400 mM, from about 50 mM to about 350 mM, from about 50 mM to about 300 mM, from about 50 mM to about 250 mM, from about 50 mM to about 200 mM, from about 50 mM to about 150 mM, from about 50 mM to about 125 mM, from about 50 mM to about 100 mM, from about 50 mM to about 90 mM, from about 50 mM to about 80 mM, from about 50 mM to about 70 mM, from about 50 mM to about 60 mM, from about 60 mM to about 450 mM, from about 60 mM to about 400 mM, from about 60 mM to about 350 mM, from about 60 mM to about 300 mM, from about 60 mM to about 250 mM, from about 60 mM to about 200 mM, from about 60 mM to about 150 mM, from about 60 mM to about 125 mM, from about 60 mM to about 100 mM, from about 60 mM to about 90 mM, from about 60 mM to about 80 mM, from about 60 mM to about 70 mM, from about 70 mM to about 450 mM, from about 70 mM to about 400 mM, from about 70 mM to about 350 mM, from about 70 mM to about 300 mM, from about 70 mM to about 250 mM, from about 70 mM to about 200 mM, from about 70 mM to about 150 mM, from about 70 mM to about 100 mM, from about 70 mM to about 90 mM, from about 70 mM to about 80 mM, from about 80 mM to about 450 mM, from about 80 mM to about 400 mM, from about 80 mM to about 350 mM, from about 80 mM to about 300 mM, from about 80 mM to about 250 mM, from about 80 mM to about 200 mM, from about 80 mM to about 150 mM, from about 80 mM to about 100 mM, from about 80 mM to about 90 mM, from about 90 mM to about 450 mM, from about 90 mM to about 400 mM, from about 90 mM to about 350 mM, from about 90 mM to about 300 mM, from about 90 mM to about 250 mM, from about 90 mM to about 200 mM, from about 90 mM to about 150 mM, from about 90 mM to about 100 mM, from about 100 mM to about 450 mM, from about 100 mM to about 400 mM, from about 100 mM to about 350 mM, from about 100 mM to about 300 mM, from about 100 mM to about 250 mM, from about 100 mM to about 200 mM, from about 100 mM to about 150 mM, from about 150 mM to about 450 mM, from about 150 mM to about 400 mM, from about 150 mM to about 350 mM, from about 150 mM to about 300 mM, from about 150 mM to about 250 mM, from about 150 mM to about 200 mM, from about 200 mM to about 450 mM, from about 200 mM to about 400 mM, from about 200 mM to about 350 mM, from about 200 mM to about 300 mM, from about 200 mM to about 250 mM, from about 250 mM to about 450 mM, from about 250 mM to about 400 mM, from about 250 mM to about 350 mM, from about 250 mM to about 300 mM, from about 300 mM to about 450 mM, from about 300 mM to about 400 mM, from about 300 mM to about 350 mM, from about 350 mM to about 450 mM, from about 350 mM to about 400 mM, or from about 400 mM to about 450 mM. In certain embodiments, the concentration of the pharmaceutically acceptable buffer in a liquid pharmaceutical formulation described herein can be from about 50 mM to about 125 mM.

In certain embodiments, the concentration of the pharmaceutically acceptable buffer in a liquid pharmaceutical formulation described herein can be about 10 mM, about 15 mM, about 20 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 150 mM, about 160 mM, about 170 mM, about 180 mM, about 190 mM, about 200 mM, about 225 mM, about 250 mM, about 275 mM, about 300 mM, about 325 mM, about 350 mM, about 375 mM, about 400 mM, about 425 mM, about 450 mM, about 475 mM, or about 500 mM. In certain embodiments, the concentration of the pharmaceutically acceptable buffer in a liquid pharmaceutical formulation described herein can be about 50 mM. In certain embodiments, the concentration of the pharmaceutically acceptable buffer in a liquid pharmaceutical formulation described herein can be about 125 mM.

In certain embodiments, the concentration of the pharmaceutically acceptable buffer in a liquid pharmaceutical formulation described herein can be less than or equal to about 10 mM, less than or equal to about 20 mM, less than or equal to about 30 mM, less than or equal to about 40 mM, less than or equal to about 50 mM, less than or equal to about 60 mM, less than or equal to about 70 mM, less than or equal to about 80 mM, less than or equal to about 90 mM, less than or equal to about 100 mM, less than or equal to about 125 mM, less than or equal to about 150 mM, less than or equal to about 175 mM, less than or equal to about 200 mM, less than or equal to about 225 mM, less than or equal to about 250 mM, less than or equal to about 275 mM, less than or equal to about 300 mM, less than or equal to about 325 mM, less than or equal to about 350 mM, less than or equal to about 375 mM, less than or equal to about 400 mM, less than or equal to about 425 mM, less than or equal to about 450 mM, less than or equal to about 475 mM, or less than or equal to about 500 mM.

In certain embodiments, the pharmaceutically acceptable buffer comprises a buffering agent selected from the group consisting of histidine, a citrate salt, sodium phosphate, potassium phosphate, tromethamine or a pharmaceutically acceptable salt thereof, and any combination thereof. In certain embodiments, the buffering agent is histidine. In certain embodiments, the buffering agent is a citrate salt. In certain embodiments, the buffering agent is sodium phosphate. In certain embodiments, the buffering agent is potassium phosphate. In certain embodiments, the buffering agent is tromethamine or a pharmaceutically acceptable salt thereof. In some embodiments, the pharmaceutically acceptable salt of tromethamine is tromethamine hydrochloride.

In certain embodiments, the concentration of tromethamine or a pharmaceutically acceptable salt thereof in a liquid pharmaceutical formulation described herein can be from about 10 mM to about 250 mM, from about 15 mM to about 250 mM, from about 20 mM to about 250 mM, from about 25 mM to about 250 mM, from about 30 mM to about 250 mM, from about 40 mM to about 250 mM, from about 50 mM to about 250 mM, from about 60 mM to about 250 mM, from about 70 mM to about 250 mM, from about 80 mM to about 250 mM, from about 90 mM to about 250 mM, from about 100 mM to about 250 mM, from about 125 mM to about 250 mM, from about 150 mM to about 250 mM, from about 175 mM to about 250 mM, from about 200 mM to about 250 mM, from about 225 mM to about 250 mM, from about 10 mM to about 225 mM, from about 10 mM to about 200 mM, from about 10 mM to about 175 mM, from about 10 mM to about 150 mM, from about 10 mM to about 125 mM, from about 10 mM to about 100 mM, from about 10 mM to about 90 mM, from about 10 mM to about 80 mM, from about 10 mM to about 70 mM, from about 10 mM to about 60 mM, from about 10 mM to about 50 mM, from about 10 mM to about 40 mM, from about 10 mM to about 30 mM, from about 10 mM to about 25 mM, from about 10 mM to about 20 mM, from about 10 mM to about 15 mM, from about 15 mM to about 225 mM, from about 15 mM to about 200 mM, from about 15 mM to about 175 mM, from about 15 mM to about 150 mM, from about 15 mM to about 125 mM, from about 15 mM to about 100 mM, from about 15 mM to about 90 mM, from about 15 mM to about 80 mM, from about 15 mM to about 70 mM, from about 15 mM to about 60 mM, from about 15 mM to about 50 mM, from about 15 mM to about 40 mM, from about 15 mM to about 30 mM, from about 15 mM to about 25 mM, from about 15 mM to about 20 mM, from about 20 mM to about 225 mM, from about 20 mM to about 200 mM, from about 20 mM to about 175 mM, from about 20 mM to about 150 mM, from about 20 mM to about 125 mM, from about 20 mM to about 100 mM, from about 20 mM to about 90 mM, from about 20 mM to about 80 mM, from about 20 mM to about 70 mM, from about 20 mM to about 60 mM, from about 20 mM to about 50 mM, from about 20 mM to about 40 mM, from about 20 mM to about 30 mM, from about 20 mM to about 25 mM, from about 25 mM to about 225 mM, from about 25 mM to about 200 mM, from about 25 mM to about 175 mM, from about 25 mM to about 150 mM, from about 25 mM to about 125 mM, from about 25 mM to about 100 mM, from about 25 mM to about 90 mM, from about 25 mM to about 80 mM, from about 25 mM to about 70 mM, from about 25 mM to about 60 mM, from about 25 mM to about 50 mM, from about 25 mM to about 40 mM, from about 25 mM to about 30 mM, from about 30 mM to about 225 mM, from about 30 mM to about 200 mM, from about 30 mM to about 175 mM, from about 30 mM to about 150 mM, from about 30 mM to about 125 mM, from about 30 mM to about 100 mM, from about 30 mM to about 90 mM, from about 30 mM to about 80 mM, from about 30 mM to about 70 mM, from about 30 mM to about 60 mM, from about 30 mM to about 50 mM, from about 30 mM to about 40 mM, from about 40 mM to about 225 mM, from about 40 mM to about 200 mM, from about 40 mM to about 175 mM, from about 40 mM to about 150 mM, from about 40 mM to about 125 mM, from about 40 mM to about 100 mM, from about 40 mM to about 90 mM, from about 40 mM to about 80 mM, from about 40 mM to about 70 mM, from about 40 mM to about 60 mM, from about 40 mM to about 50 mM, from about 50 mM to about 225 mM, from about 50 mM to about 200 mM, from about 50 mM to about 175 mM, from about 50 mM to about 150 mM, from about 50 mM to about 125 mM, from about 50 mM to about 100 mM, from about 50 mM to about 90 mM, from about 50 mM to about 80 mM, from about 50 mM to about 70 mM, from about 50 mM to about 60 mM, from about 60 mM to about 225 mM, from about 60 mM to about 200 mM, from about 60 mM to about 175 mM, from about 60 mM to about 150 mM, from about 60 mM to about 125 mM, from about 60 mM to about 100 mM, from about 60 mM to about 90 mM, from about 60 mM to about 80 mM, from about 60 mM to about 70 mM, from about 60 mM to about 60 mM, from about 70 mM to about 225 mM, from about 70 mM to about 200 mM, from about 70 mM to about 175 mM, from about 70 mM to about 150 mM, from about 70 mM to about 125 mM, from about 70 mM to about 100 mM, from about 70 mM to about 90 mM, from about 70 mM to about 80 mM, from about 80 mM to about 225 mM, from about 80 mM to about 200 mM, from about 80 mM to about 175 mM, from about 80 mM to about 150 mM, from about 80 mM to about 125 mM, from about 80 mM to about 100 mM, from about 80 mM to about 90 mM, from about 90 mM to about 225 mM, from about 90 mM to about 200 mM, from about 90 mM to about 175 mM, from about 90 mM to about 150 mM, from about 90 mM to about 125 mM, from about 90 mM to about 100 mM, from about 100 mM to about 225 mM, from about 100 mM to about 200 mM, from about 100 mM to about 175 mM, from about 100 mM to about 150 mM, from about 100 mM to about 125 mM, from about 125 mM to about 225 mM, from about 125 mM to about 200 mM, from about 125 mM to about 175 mM, from about 125 mM to about 150 mM, from about 150 mM to about 225 mM, from about 150 mM to about 200 mM, from about 150 mM to about 175 mM, from about 150 mM to about 250 mM, from about 175 mM to about 225 mM, from about 175 mM to about 200 mM, or from about 200 mM to about 225 mM. In certain embodiments, the concentration of tromethamine or a pharmaceutically acceptable salt thereof in a liquid pharmaceutical formulation described herein is from about 10 mM to about 200 mM. In certain embodiments, the concentration of tromethamine or a pharmaceutically acceptable salt thereof in a liquid pharmaceutical formulation described herein is from about 25 mM to about 150 mM. In certain embodiments, the concentration of tromethamine or a pharmaceutically acceptable salt thereof in a liquid pharmaceutical formulation described herein is from about 50 mM to about 125 mM.

In certain embodiments, the concentration of tromethamine or a pharmaceutically acceptable salt thereof in a liquid pharmaceutical formulation described herein can be about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 32 mM, about 34 mM, about 36 mM, about 38 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 140 mM, about 150 mM, about 160 mM, about 170 mM, about 180 mM, about 190 mM, about 200 mM, about 225 mM, or about 250 mM. In certain embodiments, the concentration of tromethamine or a pharmaceutically acceptable salt thereof in a liquid pharmaceutical formulation described herein can be about 50 mM. In certain embodiments, the concentration of tromethamine or a pharmaceutically acceptable salt thereof in a liquid pharmaceutical formulation described herein can be about 125 mM.

In certain embodiments, the concentration of tromethamine or a pharmaceutically acceptable salt thereof in a liquid pharmaceutical formulation described herein can be less than or equal to about 10 mM, less than or equal to about 20 mM, less than or equal to about 30 mM, less than or equal to about 40 mM, less than or equal to about 50 mM, less than or equal to about 60 mM, less than or equal to about 70 mM, less than or equal to about 80 mM, less than or equal to about 90 mM, less than or equal to about 100 mM, less than or equal to about 125 mM, less than or equal to about 150 mM, less than or equal to about 175 mM, less than or equal to about 200 mM, less than or equal to about 225 mM, or less than or equal to about 250 mM.

### (ii) pH Modifiers

In certain embodiments, a liquid pharmaceutical formulation described herein further comprises a pharmaceutically acceptable pH adjuster.

Exemplary pharmaceutically acceptable pH adjusters include, but are not limited to, one or more of acetic acid, citric acid, fumaric acid, hydrochloric acid, malic acid, nitric acid, phosphoric acid, propionic acid, sulfuric acid, tartaric acid, ammonia solution, ammonium carbonate, diethanolamine, potassium hydroxide, sodium bicarbonate, sodium borate, sodium carbonate, sodium hydroxide, and trolamine.

In certain embodiments, the pharmaceutically acceptable pH adjuster is selected from the group consisting of potassium hydroxide, sodium hydroxide, hydrochloric acid, and combinations thereof. In certain embodiments, the pharmaceutically acceptable pH adjuster is hydrochloric acid and sodium hydroxide. In certain embodiments, the pharmaceutically acceptable pH adjuster is sodium hydroxide. In certain embodiments, the pharmaceutically acceptable pH adjuster is potassium hydroxide. In certain embodiments, the pharmaceutically acceptable pH adjuster is hydrochloric acid.

### Antioxidants

In certain embodiments, the liquid pharmaceutical formulation described herein can further comprise an antioxidant.

In certain embodiments, the amount of the antioxidant in a liquid pharmaceutical formulation described herein can be from about 0.01% (w/w) to about 2% (w/w), from about 0.05% (w/w) to about 2% (w/w), from about 0.1% (w/w) to about 2% (w/w), from about 0.2% (w/w) to about 2% (w/w), from about 0.3% (w/w) to about 2% (w/w), from about 0.4% (w/w) to about 2% (w/w), from about 0.5% (w/w) to about 2% (w/w), from about 0.6% (w/w) to about 2% (w/w), from about 0.7% (w/w) to about 2% (w/w), from about 0.8% (w/w) to about 2% (w/w), from about 0.9% (w/w) to about 2% (w/w), from about 1% (w/w) to about 2% (w/w), from about 1.1% (w/w) to about 2% (w/w), from about 1.2% (w/w) to about 2% (w/w), from about 1.3% (w/w) to about 2% (w/w), from about 1.4% (w/w) to about 2% (w/w), from about 1.5% (w/w) to about 2% (w/w), from about 1.6% (w/w) to about 2% (w/w), from about 1.7% (w/w) to about 2% (w/w), from about 1.8% (w/w) to about 2% (w/w), from about 1.9% (w/w) to about 2% (w/w), from about 0.01% (w/w) to about 1.9% (w/w), from about 0.01% (w/w) to about 1.8% (w/w), from about 0.01% (w/w) to about 1.7% (w/w), from about 0.01% (w/w) to about 1.6% (w/w), from about 0.01% (w/w) to about 1.5% (w/w), from about 0.01% (w/w) to about 1.4% (w/w), from about 0.01% (w/w) to about 1.3% (w/w), from about 0.01% (w/w) to about 1.2% (w/w), from about 0.01% (w/w) to about 1.1% (w/w), from about 0.01% (w/w) to about 1% (w/w), from about 0.01% (w/w) to about 0.9% (w/w), from about 0.01% (w/w) to about 0.8% (w/w), from about 0.01% (w/w) to about 0.7% (w/w), from about 0.01% (w/w) to about 0.6% (w/w), from about 0.01% (w/w) to about 0.5% (w/w), from about 0.01% (w/w) to about 0.4% (w/w), from about 0.01% (w/w) to about 0.3% (w/w), from about 0.01% (w/w) to about 0.2% (w/w), from about 0.01% (w/w) to about 0.1% (w/w), from about 0.01% (w/w) to about 0.05% (w/w), from about 0.05% (w/w) to about 1.9% (w/w), from about 0.05% (w/w) to about 1.8% (w/w), from about 0.05% (w/w) to about 1.7% (w/w), from about 0.05% (w/w) to about 1.6% (w/w), from about 0.05% (w/w) to about 1.5% (w/w), from about 0.05% (w/w) to about 1.4% (w/w), from about 0.05% (w/w) to about 1.3% (w/w), from about 0.05% (w/w) to about 1.2% (w/w), from about 0.05% (w/w) to about 1.1% (w/w), from about 0.05% (w/w) to about 1% (w/w), from about 0.05% (w/w) to about 0.9% (w/w), from about 0.05% (w/w) to about 0.8% (w/w), from about 0.05% (w/w) to about 0.7% (w/w), from about 0.05% (w/w) to about 0.6% (w/w), from about 0.05% (w/w) to about 0.5% (w/w), from about 0.05% (w/w) to about 0.4% (w/w), from about 0.05% (w/w) to about 0.3% (w/w), from about 0.05% (w/w) to about 0.2% (w/w), from about 0.05% (w/w) to about 0.1% (w/w), from about 0.1% (w/w) to about 1.9% (w/w), from about 0.1% (w/w) to about 1.8% (w/w), from about 0.1% (w/w) to about 1.7% (w/w), from about 0.1% (w/w) to about 1.6% (w/w), from about 0.1% (w/w) to about 1.5% (w/w), from about 0.1% (w/w) to about 1.4% (w/w), from about 0.1% (w/w) to about 1.3% (w/w), from about 0.1% (w/w) to about 1.2% (w/w), from about 0.1% (w/w) to about 1.1% (w/w), from about 0.1% (w/w) to about 1% (w/w), from about 0.1% (w/w) to about 0.9% (w/w), from about 0.1% (w/w) to about 0.8% (w/w), from about 0.1% (w/w) to about 0.7% (w/w), from about 0.1% (w/w) to about 0.6% (w/w), from about 0.1% (w/w) to about 0.5% (w/w), from about 0.1% (w/w) to about 0.4% (w/w), from about 0.1% (w/w) to about 0.3% (w/w), from about 0.1% (w/w) to about 0.2% (w/w), from about 0.2% (w/w) to about 1.9% (w/w), from about 0.2% (w/w) to about 1.8% (w/w), from about 0.2% (w/w) to about 1.7% (w/w), from about 0.2% (w/w) to about 1.6% (w/w), from about 0.2% (w/w) to about 1.5% (w/w), from about 0.2% (w/w) to about 1.4% (w/w), from about 0.2% (w/w) to about 1.3% (w/w), from about 0.2% (w/w) to about 1.2% (w/w), from about 0.2% (w/w) to about 1.1% (w/w), from about 0.2% (w/w) to about 1% (w/w), from about 0.2% (w/w) to about 0.9% (w/w), from about 0.2% (w/w) to about 0.8% (w/w), from about 0.2% (w/w) to about 0.7% (w/w), from about 0.2% (w/w) to about 0.6% (w/w), from about 0.2% (w/w) to about 0.5% (w/w), from about 0.2% (w/w) to about 0.4% (w/w), from about 0.2% (w/w) to about 0.3% (w/w), from about 0.3% (w/w) to about 1.9% (w/w), from about 0.3% (w/w) to about 1.8% (w/w), from about 0.3% (w/w) to about 1.7% (w/w), from about 0.3% (w/w) to about 1.6% (w/w), from about 0.3% (w/w) to about 1.5% (w/w), from about 0.3% (w/w) to about 1.4% (w/w), from about 0.3% (w/w) to about 1.3% (w/w), from about 0.3% (w/w) to about 1.2% (w/w), from about 0.3% (w/w) to about 1.1% (w/w), from about 0.3% (w/w) to about 1% (w/w), from about 0.3% (w/w) to about 0.9% (w/w), from about 0.3% (w/w) to about 0.8% (w/w), from about 0.3% (w/w) to about 0.7% (w/w), from about 0.3% (w/w) to about 0.6% (w/w), from about 0.3% (w/w) to about 0.5% (w/w), from about 0.3% (w/w) to about 0.4% (w/w), from about 0.4% (w/w) to about 1.9% (w/w), from about 0.4% (w/w) to about 1.8% (w/w), from about 0.4% (w/w) to about 1.7% (w/w), from about 0.4% (w/w) to about 1.6% (w/w), from about 0.4% (w/w) to about 1.5% (w/w), from about 0.4% (w/w) to about 1.4% (w/w), from about 0.4% (w/w) to about 1.3% (w/w), from about 0.4% (w/w) to about 1.2% (w/w), from about 0.4% (w/w) to about 1.1% (w/w), from about 0.4% (w/w) to about 1% (w/w), from about 0.4% (w/w) to about 0.9% (w/w), from about 0.4% (w/w) to about 0.8% (w/w), from about 0.4% (w/w) to about 0.7% (w/w), from about 0.4% (w/w) to about 0.6% (w/w), from about 0.4% (w/w) to about 0.5% (w/w), from about 0.5% (w/w) to about 1.9% (w/w), from about 0.5% (w/w) to about 1.8% (w/w), from about 0.5% (w/w) to about 1.7% (w/w), from about 0.5% (w/w) to about 1.6% (w/w), from about 0.5% (w/w) to about 1.5% (w/w), from about 0.5% (w/w) to about 1.4% (w/w), from about 0.5% (w/w) to about 1.3% (w/w), from about 0.5% (w/w) to about 1.2% (w/w), from about 0.5% (w/w) to about 1.1% (w/w), from about 0.5% (w/w) to about 1% (w/w), from about 0.5% (w/w) to about 0.9% (w/w), from about 0.5% (w/w) to about 0.8% (w/w), from about 0.5% (w/w) to about 0.7% (w/w), from about 0.5% (w/w) to about 0.6% (w/w), from about 0.6% (w/w) to about 1.9% (w/w), from about 0.6% (w/w) to about 1.8% (w/w), from about 0.6% (w/w) to about 1.7% (w/w), from about 0.6% (w/w) to about 1.6% (w/w), from about 0.6% (w/w) to about 1.5% (w/w), from about 0.6% (w/w) to about 1.4% (w/w), from about 0.6% (w/w) to about 1.3% (w/w), from about 0.6% (w/w) to about 1.2% (w/w), from about 0.6% (w/w) to about 1.1% (w/w), from about 0.6% (w/w) to about 1% (w/w), from about 0.6% (w/w) to about 0.9% (w/w), from about 0.6% (w/w) to about 0.8% (w/w), from about 0.6% (w/w) to about 0.7% (w/w), from about 0.7% (w/w) to about 1.9% (w/w), from about 0.7% (w/w) to about 1.8% (w/w), from about 0.7% (w/w) to about 1.7% (w/w), from about 0.7% (w/w) to about 1.6% (w/w), from about 0.7% (w/w) to about 1.5% (w/w), from about 0.7% (w/w) to about 1.4% (w/w), from about 0.7% (w/w) to about 1.3% (w/w), from about 0.7% (w/w) to about 1.2% (w/w), from about 0.7% (w/w) to about 1.1% (w/w), from about 0.7% (w/w) to about 1% (w/w), from about 0.7% (w/w) to about 0.9% (w/w), from about 0.7% (w/w) to about 0.8% (w/w), from about 0.8% (w/w) to about 1.9% (w/w), from about 0.8% (w/w) to about 1.8% (w/w), from about 0.8% (w/w) to about 1.7% (w/w), from about 0.8% (w/w) to about 1.6% (w/w), from about 0.8% (w/w) to about 1.5% (w/w), from about 0.8% (w/w) to about 1.4% (w/w), from about 0.8% (w/w) to about 1.3% (w/w), from about 0.8% (w/w) to about 1.2% (w/w), from about 0.8% (w/w) to about 1.1% (w/w), from about 0.8% (w/w) to about 1% (w/w), from about 0.8% (w/w) to about 0.9% (w/w), from about 0.9% (w/w) to about 1.9% (w/w), from about 0.9% (w/w) to about 1.8% (w/w), from about 0.9% (w/w) to about 1.7% (w/w), from about 0.9% (w/w) to about 1.6% (w/w), from about 0.9% (w/w) to about 1.5% (w/w), from about 0.9% (w/w) to about 1.4% (w/w), from about 0.9% (w/w) to about 1.3% (w/w), from about 0.9% (w/w) to about 1.2% (w/w), from about 0.9% (w/w) to about 1.1% (w/w), from about 0.9% (w/w) to about 1% (w/w), from about 1% (w/w) to about 1.9% (w/w), from about 1% (w/w) to about 1.8% (w/w), from about 1% (w/w) to about 1.7% (w/w), from about 1% (w/w) to about 1.6% (w/w), from about 1% (w/w) to about 1.5% (w/w), from about 1% (w/w) to about 1.4% (w/w), from about 1% (w/w) to about 1.3% (w/w), from about 1% (w/w) to about 1.2% (w/w), from about 1% (w/w) to about 1.1% (w/w), from about 1.1% (w/w) to about 1.9% (w/w), from about 1.1% (w/w) to about 1.8% (w/w), from about 1.1% (w/w) to about 1.7% (w/w), from about 1.1% (w/w) to about 1.6% (w/w), from about 1.1% (w/w) to about 1.5% (w/w), from about 1.1% (w/w) to about 1.4% (w/w), from about 1.1% (w/w) to about 1.3% (w/w), from about 1.1% (w/w) to about 1.2% (w/w), from about 1.2% (w/w) to about 1.9% (w/w), from about 1.2% (w/w) to about 1.8% (w/w), from about 1.2% (w/w) to about 1.7% (w/w), from about 1.2% (w/w) to about 1.6% (w/w), from about 1.2% (w/w) to about 1.5% (w/w), from about 1.2% (w/w) to about 1.4% (w/w), from about 1.2% (w/w) to about 1.3% (w/w), from about 1.3% (w/w) to about 1.9% (w/w), from about 1.3% (w/w) to about 1.8% (w/w), from about 1.3% (w/w) to about 1.7% (w/w), from about 1.3% (w/w) to about 1.6% (w/w), from about 1.3% (w/w) to about 1.5% (w/w), from about 1.3% (w/w) to about 1.4% (w/w), from about 1.4% (w/w) to about 1.9% (w/w), from about 1.4% (w/w) to about 1.8% (w/w), from about 1.4% (w/w) to about 1.7% (w/w), from about 1.4% (w/w) to about 1.6% (w/w), from about 1.4% (w/w) to about 1.5% (w/w), from about 1.5% (w/w) to about 1.9% (w/w), from about 1.5% (w/w) to about 1.8% (w/w), from about 1.5% (w/w) to about 1.7% (w/w), from about 1.5% (w/w) to about 1.6% (w/w), from about 1.6% (w/w) to about 1.9% (w/w), from about 1.6% (w/w) to about 1.8% (w/w), from about 1.6% (w/w) to about 1.7% (w/w), from about 1.7% (w/w) to about 1.9% (w/w), from about 1.7% (w/w) to about 1.8% (w/w), or from about 1.8% (w/w) to about 1.9% (w/w). In certain embodiments, the amount of the antioxidant in a liquid pharmaceutical formulation described herein can be from about 0.01% (w/w) to about 0.3% (w/w). In certain embodiments, the amount of the antioxidant in a liquid pharmaceutical formulation described herein can be from about 0.05% (w/w) to about 2% (w/w).

In certain embodiments, the amount of the antioxidant in a liquid pharmaceutical formulation described herein can be about 0.05% (w/w), about 0.1% (w/w), about 0.2% (w/w), about 0.3% (w/w), about 0.4% (w/w), about 0.5% (w/w), about 0.6% (w/w), about 0.7% (w/w), about 0.8% (w/w), about 0.9% (w/w), about 1.0% (w/w), about 1.1% (w/w), about 1.2% (w/w), about 1.3% (w/w), about 1.4% (w/w), about 1.5% (w/w), about 1.6% (w/w), about 1.7% (w/w), about 1.8% (w/w), about 1.9% (w/w), or about 2.0% (w/w). In certain embodiments, the amount of the antioxidant in a liquid pharmaceutical formulation described herein can be about 0.3% (w/w).

In certain embodiments, the antioxidant is selected from the group consisting of sodium metabisulfite, sodium bisulfite monothioglycerol, methionine, ethylenediaminetetraacetic acid (EDTA), and combinations thereof. In certain embodiments, the antioxidant is sodium metabisulfite. In certain embodiments, the antioxidant is sodium bisulfite monothioglycerol. In certain embodiments, the antioxidant is methionine. In certain embodiments, the antioxidant is EDTA.

In certain embodiments, the amount of sodium metabisulfite in a liquid pharmaceutical formulation described herein can be from about 0.01% (w/w) to about 2% (w/w), from about 0.05% (w/w) to about 2% (w/w), from about 0.1% (w/w) to about 2% (w/w), from about 0.2% (w/w) to about 2% (w/w), from about 0.3% (w/w) to about 2% (w/w), from about 0.4% (w/w) to about 2% (w/w), from about 0.5% (w/w) to about 2% (w/w), from about 0.6% (w/w) to about 2% (w/w), from about 0.7% (w/w) to about 2% (w/w), from about 0.8% (w/w) to about 2% (w/w), from about 0.9% (w/w) to about 2% (w/w), from about 1% (w/w) to about 2% (w/w), from about 1.1% (w/w) to about 2% (w/w), from about 1.2% (w/w) to about 2% (w/w), from about 1.3% (w/w) to about 2% (w/w), from about 1.4% (w/w) to about 2% (w/w), from about 1.5% (w/w) to about 2% (w/w), from about 1.6% (w/w) to about 2% (w/w), from about 1.7% (w/w) to about 2% (w/w), from about 1.8% (w/w) to about 2% (w/w), from about 1.9% (w/w) to about 2% (w/w), from about 0.01% (w/w) to about 1.9% (w/w), from about 0.01% (w/w) to about 1.8% (w/w), from about 0.01% (w/w) to about 1.7% (w/w), from about 0.01% (w/w) to about 1.6% (w/w), from about 0.01% (w/w) to about 1.5% (w/w), from about 0.01% (w/w) to about 1.4% (w/w), from about 0.01% (w/w) to about 1.3% (w/w), from about 0.01% (w/w) to about 1.2% (w/w), from about 0.01% (w/w) to about 1.1% (w/w), from about 0.01% (w/w) to about 1% (w/w), from about 0.01% (w/w) to about 0.9% (w/w), from about 0.01% (w/w) to about 0.8% (w/w), from about 0.01% (w/w) to about 0.7% (w/w), from about 0.01% (w/w) to about 0.6% (w/w), from about 0.01% (w/w) to about 0.5% (w/w), from about 0.01% (w/w) to about 0.4% (w/w), from about 0.01% (w/w) to about 0.3% (w/w), from about 0.01% (w/w) to about 0.2% (w/w), from about 0.01% (w/w) to about 0.1% (w/w), from about 0.01% (w/w) to about 0.05% (w/w), from about 0.05% (w/w) to about 1.9% (w/w), from about 0.05% (w/w) to about 1.8% (w/w), from about 0.05% (w/w) to about 1.7% (w/w), from about 0.05% (w/w) to about 1.6% (w/w), from about 0.05% (w/w) to about 1.5% (w/w), from about 0.05% (w/w) to about 1.4% (w/w), from about 0.05% (w/w) to about 1.3% (w/w), from about 0.05% (w/w) to about 1.2% (w/w), from about 0.05% (w/w) to about 1.1% (w/w), from about 0.05% (w/w) to about 1% (w/w), from about 0.05% (w/w) to about 0.9% (w/w), from about 0.05% (w/w) to about 0.8% (w/w), from about 0.05% (w/w) to about 0.7% (w/w), from about 0.05% (w/w) to about 0.6% (w/w), from about 0.05% (w/w) to about 0.5% (w/w), from about 0.05% (w/w) to about 0.4% (w/w), from about 0.05% (w/w) to about 0.3% (w/w), from about 0.05% (w/w) to about 0.2% (w/w), from about 0.05% (w/w) to about 0.1% (w/w), from about 0.1% (w/w) to about 1.9% (w/w), from about 0.1% (w/w) to about 1.8% (w/w), from about 0.1% (w/w) to about 1.7% (w/w), from about 0.1% (w/w) to about 1.6% (w/w), from about 0.1% (w/w) to about 1.5% (w/w), from about 0.1% (w/w) to about 1.4% (w/w), from about 0.1% (w/w) to about 1.3% (w/w), from about 0.1% (w/w) to about 1.2% (w/w), from about 0.1% (w/w) to about 1.1% (w/w), from about 0.1% (w/w) to about 1% (w/w), from about 0.1% (w/w) to about 0.9% (w/w), from about 0.1% (w/w) to about 0.8% (w/w), from about 0.1% (w/w) to about 0.7% (w/w), from about 0.1% (w/w) to about 0.6% (w/w), from about 0.1% (w/w) to about 0.5% (w/w), from about 0.1% (w/w) to about 0.4% (w/w), from about 0.1% (w/w) to about 0.3% (w/w), from about 0.1% (w/w) to about 0.2% (w/w), from about 0.2% (w/w) to about 1.9% (w/w), from about 0.2% (w/w) to about 1.8% (w/w), from about 0.2% (w/w) to about 1.7% (w/w), from about 0.2% (w/w) to about 1.6% (w/w), from about 0.2% (w/w) to about 1.5% (w/w), from about 0.2% (w/w) to about 1.4% (w/w), from about 0.2% (w/w) to about 1.3% (w/w), from about 0.2% (w/w) to about 1.2% (w/w), from about 0.2% (w/w) to about 1.1% (w/w), from about 0.2% (w/w) to about 1% (w/w), from about 0.2% (w/w) to about 0.9% (w/w), from about 0.2% (w/w) to about 0.8% (w/w), from about 0.2% (w/w) to about 0.7% (w/w), from about 0.2% (w/w) to about 0.6% (w/w), from about 0.2% (w/w) to about 0.5% (w/w), from about 0.2% (w/w) to about 0.4% (w/w), from about 0.2% (w/w) to about 0.3% (w/w), from about 0.3% (w/w) to about 1.9% (w/w), from about 0.3% (w/w) to about 1.8% (w/w), from about 0.3% (w/w) to about 1.7% (w/w), from about 0.3% (w/w) to about 1.6% (w/w), from about 0.3% (w/w) to about 1.5% (w/w), from about 0.3% (w/w) to about 1.4% (w/w), from about 0.3% (w/w) to about 1.3% (w/w), from about 0.3% (w/w) to about 1.2% (w/w), from about 0.3% (w/w) to about 1.1% (w/w), from about 0.3% (w/w) to about 1% (w/w), from about 0.3% (w/w) to about 0.9% (w/w), from about 0.3% (w/w) to about 0.8% (w/w), from about 0.3% (w/w) to about 0.7% (w/w), from about 0.3% (w/w) to about 0.6% (w/w), from about 0.3% (w/w) to about 0.5% (w/w), from about 0.3% (w/w) to about 0.4% (w/w), from about 0.4% (w/w) to about 1.9% (w/w), from about 0.4% (w/w) to about 1.8% (w/w), from about 0.4% (w/w) to about 1.7% (w/w), from about 0.4% (w/w) to about 1.6% (w/w), from about 0.4% (w/w) to about 1.5% (w/w), from about 0.4% (w/w) to about 1.4% (w/w), from about 0.4% (w/w) to about 1.3% (w/w), from about 0.4% (w/w) to about 1.2% (w/w), from about 0.4% (w/w) to about 1.1% (w/w), from about 0.4% (w/w) to about 1% (w/w), from about 0.4% (w/w) to about 0.9% (w/w), from about 0.4% (w/w) to about 0.8% (w/w), from about 0.4% (w/w) to about 0.7% (w/w), from about 0.4% (w/w) to about 0.6% (w/w), from about 0.4% (w/w) to about 0.5% (w/w), from about 0.5% (w/w) to about 1.9% (w/w), from about 0.5% (w/w) to about 1.8% (w/w), from about 0.5% (w/w) to about 1.7% (w/w), from about 0.5% (w/w) to about 1.6% (w/w), from about 0.5% (w/w) to about 1.5% (w/w), from about 0.5% (w/w) to about 1.4% (w/w), from about 0.5% (w/w) to about 1.3% (w/w), from about 0.5% (w/w) to about 1.2% (w/w), from about 0.5% (w/w) to about 1.1% (w/w), from about 0.5% (w/w) to about 1% (w/w), from about 0.5% (w/w) to about 0.9% (w/w), from about 0.5% (w/w) to about 0.8% (w/w), from about 0.5% (w/w) to about 0.7% (w/w), from about 0.5% (w/w) to about 0.6% (w/w), from about 0.6% (w/w) to about 1.9% (w/w), from about 0.6% (w/w) to about 1.8% (w/w), from about 0.6% (w/w) to about 1.7% (w/w), from about 0.6% (w/w) to about 1.6% (w/w), from about 0.6% (w/w) to about 1.5% (w/w), from about 0.6% (w/w) to about 1.4% (w/w), from about 0.6% (w/w) to about 1.3% (w/w), from about 0.6% (w/w) to about 1.2% (w/w), from about 0.6% (w/w) to about 1.1% (w/w), from about 0.6% (w/w) to about 1% (w/w), from about 0.6% (w/w) to about 0.9% (w/w), from about 0.6% (w/w) to about 0.8% (w/w), from about 0.6% (w/w) to about 0.7% (w/w), from about 0.7% (w/w) to about 1.9% (w/w), from about 0.7% (w/w) to about 1.8% (w/w), from about 0.7% (w/w) to about 1.7% (w/w), from about 0.7% (w/w) to about 1.6% (w/w), from about 0.7% (w/w) to about 1.5% (w/w), from about 0.7% (w/w) to about 1.4% (w/w), from about 0.7% (w/w) to about 1.3% (w/w), from about 0.7% (w/w) to about 1.2% (w/w), from about 0.7% (w/w) to about 1.1% (w/w), from about 0.7% (w/w) to about 1% (w/w), from about 0.7% (w/w) to about 0.9% (w/w), from about 0.7% (w/w) to about 0.8% (w/w), from about 0.8% (w/w) to about 1.9% (w/w), from about 0.8% (w/w) to about 1.8% (w/w), from about 0.8% (w/w) to about 1.7% (w/w), from about 0.8% (w/w) to about 1.6% (w/w), from about 0.8% (w/w) to about 1.5% (w/w), from about 0.8% (w/w) to about 1.4% (w/w), from about 0.8% (w/w) to about 1.3% (w/w), from about 0.8% (w/w) to about 1.2% (w/w), from about 0.8% (w/w) to about 1.1% (w/w), from about 0.8% (w/w) to about 1% (w/w), from about 0.8% (w/w) to about 0.9% (w/w), from about 0.9% (w/w) to about 1.9% (w/w), from about 0.9% (w/w) to about 1.8% (w/w), from about 0.9% (w/w) to about 1.7% (w/w), from about 0.9% (w/w) to about 1.6% (w/w), from about 0.9% (w/w) to about 1.5% (w/w), from about 0.9% (w/w) to about 1.4% (w/w), from about 0.9% (w/w) to about 1.3% (w/w), from about 0.9% (w/w) to about 1.2% (w/w), from about 0.9% (w/w) to about 1.1% (w/w), from about 0.9% (w/w) to about 1% (w/w), from about 1% (w/w) to about 1.9% (w/w), from about 1% (w/w) to about 1.8% (w/w), from about 1% (w/w) to about 1.7% (w/w), from about 1% (w/w) to about 1.6% (w/w), from about 1% (w/w) to about 1.5% (w/w), from about 1% (w/w) to about 1.4% (w/w), from about 1% (w/w) to about 1.3% (w/w), from about 1% (w/w) to about 1.2% (w/w), from about 1% (w/w) to about 1.1% (w/w), from about 1.1% (w/w) to about 1.9% (w/w), from about 1.1% (w/w) to about 1.8% (w/w), from about 1.1% (w/w) to about 1.7% (w/w), from about 1.1% (w/w) to about 1.6% (w/w), from about 1.1% (w/w) to about 1.5% (w/w), from about 1.1% (w/w) to about 1.4% (w/w), from about 1.1% (w/w) to about 1.3% (w/w), from about 1.1% (w/w) to about 1.2% (w/w), from about 1.2% (w/w) to about 1.9% (w/w), from about 1.2% (w/w) to about 1.8% (w/w), from about 1.2% (w/w) to about 1.7% (w/w), from about 1.2% (w/w) to about 1.6% (w/w), from about 1.2% (w/w) to about 1.5% (w/w), from about 1.2% (w/w) to about 1.4% (w/w), from about 1.2% (w/w) to about 1.3% (w/w), from about 1.3% (w/w) to about 1.9% (w/w), from about 1.3% (w/w) to about 1.8% (w/w), from about 1.3% (w/w) to about 1.7% (w/w), from about 1.3% (w/w) to about 1.6% (w/w), from about 1.3% (w/w) to about 1.5% (w/w), from about 1.3% (w/w) to about 1.4% (w/w), from about 1.4% (w/w) to about 1.9% (w/w), from about 1.4% (w/w) to about 1.8% (w/w), from about 1.4% (w/w) to about 1.7% (w/w), from about 1.4% (w/w) to about 1.6% (w/w), from about 1.4% (w/w) to about 1.5% (w/w), from about 1.5% (w/w) to about 1.9% (w/w), from about 1.5% (w/w) to about 1.8% (w/w), from about 1.5% (w/w) to about 1.7% (w/w), from about 1.5% (w/w) to about 1.6% (w/w), from about 1.6% (w/w) to about 1.9% (w/w), from about 1.6% (w/w) to about 1.8% (w/w), from about 1.6% (w/w) to about 1.7% (w/w), from about 1.7% (w/w) to about 1.9% (w/w), from about 1.7% (w/w) to about 1.8% (w/w), or from about 1.8% (w/w) to about 1.9% (w/w). In certain embodiments, the amount of sodium metabisulfite in a liquid pharmaceutical formulation described herein can be from about 0.01% (w/w) to about 0.3% (w/w). In certain embodiments, the amount of sodium metabisulfite in a liquid pharmaceutical formulation described herein can be from about 0.05% (w/w) to about 2% (w/w).

In certain embodiments, the amount of sodium metabisulfite in a liquid pharmaceutical formulation described herein can be about 0.05% (w/w), about 0.1% (w/w), about 0.2% (w/w), about 0.3% (w/w), about 0.4% (w/w), about 0.5% (w/w), about 0.6% (w/w), about 0.7% (w/w), about 0.8% (w/w), about 0.9% (w/w), about 1.0% (w/w), about 1.1% (w/w), about 1.2% (w/w), about 1.3% (w/w), about 1.4% (w/w), about 1.5% (w/w), about 1.6% (w/w), about 1.7% (w/w), about 1.8% (w/w), about 1.9% (w/w), or about 2.0% (w/w). In certain embodiments, the amount of the antioxidant in a liquid pharmaceutical formulation described herein can be about 0.3% (w/w).

### Osmolarity and Osmolality

In certain embodiments, a liquid pharmaceutical formulation described herein has an osmolarity in the range of from about 100 mOsm/kg to about 1600 mOsm/kg, from about 200 mOsm/kg to about 1600 mOsm/kg, from about 300 mOsm/kg to about 1600 mOsm/kg, from about 400 mOsm/kg to about 1600 mOsm/kg, from about 600 mOsm/kg to about 1600 mOsm/kg, from about 800 mOsm/kg to about 1600 mOsm/kg, from about 1200 mOsm/kg to about 1600 mOsm/kg, from about 100 mOsm/kg to about 1200 mOsm/kg, from about 100 mOsm/kg to about 800 mOsm/kg, from about 100 mOsm/kg to about 600 mOsm/kg, from about 100 mOsm/kg to about 400 mOsm/kg, from about 100 mOsm/kg to about 300 mOsm/kg, from about 100 mOsm/kg to about 200 mOsm/kg, from about 200 mOsm/kg to about 1200 mOsm/kg, from about 200 mOsm/kg to about 800 mOsm/kg, from about 200 mOsm/kg to about 600 mOsm/kg, from about 200 mOsm/kg to about 400 mOsm/kg, from about 200 mOsm/kg to about 300 mOsm/kg, from about 300 mOsm/kg to about 1200 mOsm/kg, from about 300 mOsm/kg to about 800 mOsm/kg, from about 300 mOsm/kg to about 600 mOsm/kg, from about 300 mOsm/kg to about 400 mOsm/kg, from about 400 mOsm/kg to about 1200 mOsm/kg, from about 400 mOsm/kg to about 800 mOsm/kg, from about 400 mOsm/kg to about 600 mOsm/kg, from about 600 mOsm/kg to about 1200 mOsm/kg, from about 600 mOsm/kg to about 800 mOsm/kg, or from about 800 mOsm/kg to about 1200 mOsm/kg. In some embodiments, a liquid pharmaceutical formulation described herein has an osmolarity in the range of from about 200 mOsm/kg to about 400 mOsm/kg. In some embodiments, a liquid pharmaceutical formulation described herein has an osmolarity in the range of from about 300 mOsm/kg to about 600 mOsm/kg. In certain embodiments, a liquid pharmaceutical formulation described herein has an osmolarity in the range of from about 300 mOsm/kg to about 450 mOsm/kg.

In certain embodiments, a liquid pharmaceutical formulation described herein has an osmolarity in the range of about 100 mOsm/kg to about 1600 mOsm/kg. In certain embodiments, a liquid pharmaceutical formulation described herein has an osmolarity in the range of about 200 mOsm/kg to about 800 mOsm/kg. In certain embodiments, a liquid pharmaceutical formulation described herein has an osmolarity in the range of about 200 mOsm/kg to about 600 mOsm/kg. In certain embodiments, a liquid pharmaceutical formulation described herein has an osmolarity in the range of about 200 mOsm/kg to about 400 mOsm/kg. In certain embodiments, a liquid pharmaceutical formulation described herein has an osmolarity in the range of from about 275 mOsm/kg to about 400 mOsm/kg.

It should be understood that the above ranges and description of osmolarity are equally applicable to osmolality but with the units of “Osm/kg.”

In certain embodiments, a liquid pharmaceutical formulation described herein can further comprise an osmolarity or an osmolality adjuster.

In certain embodiments, the osmolarity or the osmolality adjuster is selected from the group comprising sodium chloride, potassium chloride, isosorbide, mannitol, xylitol, and combinations thereof. In certain embodiments, the osmolarity or the osmolality adjuster is sodium chloride, potassium chloride, or a combination thereof. In certain embodiments, the osmolarity or the osmolality adjuster is sodium chloride. In certain embodiments, the osmolarity or the osmolality adjuster is potassium chloride.

### Other Pharmaceutically Acceptable Excipients

In certain embodiments, a liquid pharmaceutical formulation described herein can further comprise one or more additional pharmaceutically acceptable excipients (e.g., a solubilizing agent, a pharmaceutically acceptable carrier, etc.).

In certain embodiments, the amount of the one or more additional pharmaceutically acceptable excipients in a liquid pharmaceutical formulation described herein can be from about 0.1% (w/w) to about 30% (w/w), from about 0.5% (w/w) to about 30% (w/w), from about 1% (w/w) to about 30% (w/w), from about 1.5% (w/w) to about 30% (w/w), (w/w), from about 2% (w/w) to about 30% (w/w), from about 2.5% (w/w) to about 30% (w/w), from about 3% (w/w) to about 30% (w/w), from about 3.5% (w/w) to about 30% (w/w), from about 4% (w/w) to about 30% (w/w), from about 4.5% (w/w) to about 30% (w/w), from about 5% (w/w) to about 30% (w/w), from about 10% (w/w) to about 30% (w/w), from about 15% (w/w) to about 30% (w/w), from about 20% (w/w) to about 30% (w/w), from about 25% (w/w) to about 30% (w/w), from about 0.1% (w/w) to about 25% (w/w), from about 0.1% (w/w) to about 20% (w/w), from about 0.1% (w/w) to about 15% (w/w), from about 0.1% (w/w) to about 10% (w/w), from about 0.1% (w/w) to about 5% (w/w), from about 0.1% (w/w) to about 4.5% (w/w), from about 0.1% (w/w) to about 4% (w/w), from about 0.1% (w/w) to about 3.5% (w/w), from about 0.1% (w/w) to about 3% (w/w), from about 0.1% (w/w) to about 2.5% (w/w), from about 0.1% (w/w) to about 2% (w/w), from about 0.1% (w/w) to about 1.5% (w/w), from about 0.1% (w/w) to about 1% (w/w), from about 0.1% (w/w) to about 0.5% (w/w), from about 0.5% (w/w) to about 25% (w/w), from about 0.5% (w/w) to about 20% (w/w), from about 0.5% (w/w) to about 15% (w/w), from about 0.5% (w/w) to about 10% (w/w), from about 0.5% (w/w) to about 5% (w/w), from about 0.5% (w/w) to about 4.5% (w/w), from about 0.5% (w/w) to about 4% (w/w), from about 0.5% (w/w) to about 3.5% (w/w), from about 0.5% (w/w) to about 3% (w/w), from about 0.5% (w/w) to about 2.5% (w/w), from about 0.5% (w/w) to about 2% (w/w), from about 0.5% (w/w) to about 1.5% (w/w), from about 0.5% (w/w) to about 1% (w/w), from about 1% (w/w) to about 25% (w/w), from about 1% (w/w) to about 20% (w/w), from about 1% (w/w) to about 15% (w/w), from about 1% (w/w) to about 10% (w/w), from about 1% (w/w) to about 5% (w/w), from about 1% (w/w) to about 4.5% (w/w), from about 1% (w/w) to about 4% (w/w), from about 1% (w/w) to about 3.5% (w/w), from about 1% (w/w) to about 2.5% (w/w), from about 1% (w/w) to about 2% (w/w), from about 1% (w/w) to about 1.5% (w/w), from about 1.5% (w/w) to about 25% (w/w), from about 1.5% (w/w) to about 20% (w/w), from about 1.5% (w/w) to about 15% (w/w), from about 1.5% (w/w) to about 10% (w/w), from about 1.5% (w/w) to about 5% (w/w), from about 1.5% (w/w) to about 4.5% (w/w), from about 1.5% (w/w) to about 4% (w/w), from about 1.5% (w/w) to about 3.5% (w/w), from about 1.5% (w/w) to about 3% (w/w), from about 1.5% (w/w) to about 2.5% (w/w), from about 1.5% (w/w) to about 2% (w/w), from about 2% (w/w) to about 25% (w/w), from about 2% (w/w) to about 20% (w/w), from about 2% (w/w) to about 15% (w/w), from about 2% (w/w) to about 10% (w/w), from about 2% (w/w) to about 5% (w/w), from about 2% (w/w) to about 4.5% (w/w), from about 2% (w/w) to about 4% (w/w), from about 2% (w/w) to about 3.5% (w/w), from about 2% (w/w) to about 3% (w/w), from about 2% (w/w) to about 2.5% (w/w), from about 2.5% (w/w) to about 25% (w/w), from about 2.5% (w/w) to about 20% (w/w), from about 2.5% (w/w) to about 15% (w/w), from about 2.5% (w/w) to about 10% (w/w), from about 2.5% (w/w) to about 5% (w/w), from about 2.5% (w/w) to about 4.5% (w/w), from about 2.5% (w/w) to about 4% (w/w), from about 2.5% (w/w) to about 3.5% (w/w), from about 2.5% (w/w) to about 3% (w/w), from about 3% (w/w) to about 25% (w/w), from about 3% (w/w) to about 20% (w/w), from about 3% (w/w) to about 15% (w/w), from about 3% (w/w) to about 10% (w/w), from about 3% (w/w) to about 5% (w/w), from about 3% (w/w) to about 4.5% (w/w), from about 3% (w/w) to about 4% (w/w), from about 3% (w/w) to about 3.5% (w/w), from about 3.5% (w/w) to about 25% (w/w), from about 3.5% (w/w) to about 20% (w/w), from about 3.5% (w/w) to about 15% (w/w), from about 3.5% (w/w) to about 10% (w/w), from about 3.5% (w/w) to about 5% (w/w), from about 3.5% (w/w) to about 4.5% (w/w), from about 3.5% (w/w) to about 4% (w/w), from about 4% (w/w) to about 25% (w/w), from about 4% (w/w) to about 20% (w/w), from about 4% (w/w) to about 15% (w/w), from about 4% (w/w) to about 10% (w/w), from about 4% (w/w) to about 5% (w/w), from about 4% (w/w) to about 4.5% (w/w), from about 4.5% (w/w) to about 25% (w/w), from about 4.5% (w/w) to about 20% (w/w), from about 4.5% (w/w) to about 15% (w/w), from about 4.5% (w/w) to about 10% (w/w), from about 4.5% (w/w) to about 5% (w/w), from about 5% (w/w) to about 25% (w/w), from about 5% (w/w) to about 20% (w/w), from about 5% (w/w) to about 15% (w/w), from about 5% (w/w) to about 10% (w/w), from about 10% (w/w) to about 25% (w/w), from about 10% (w/w) to about 20% (w/w), from about 10% (w/w) to about 15% (w/w), from about 15% (w/w) to about 25% (w/w), from about 15% (w/w) to about 20% (w/w), or from about 20% (w/w) to about 25% (w/w).

In certain embodiments, the amount of the one or more additional pharmaceutically acceptable excipients in a liquid pharmaceutical formulation described herein can be about 0.1% (w/w), about 0.25% (w/w), about 0.5% (w/w), about 0.75% (w/w), about 1% (w/w), about 1.25% (w/w), about 1.5% (w/w), about 1.75% (w/w), about 2% (w/w), about 2.25% (w/w), about 2.5% (w/w), about 2.75% (w/w), about 3% (w/w), about 3.5% (w/w), about 4% (w/w), about 4.5% (w/w), about 5% (w/w), about 6% (w/w), about 7% (w/w), about 8% (w/w), about 9% (w/w), about 10% (w/w), about 11% (w/w), about 12% (w/w), about 13% (w/w), about 14% (w/w), about 15% (w/w), about 16% (w/w), about 17% (w/w), about 18% (w/w), about 19% (w/w), about 20% (w/w), about 21% (w/w), about 22% (w/w), about 23% (w/w), about 24% (w/w), about 25% (w/w), about 26% (w/w), about 27% (w/w), about 28% (w/w), about 29% (w/w), or about 30% (w/w). In certain embodiments, the amount of the one or more additional pharmaceutically acceptable excipients in a liquid pharmaceutical formulation described herein can be about 1% (w/w). In certain embodiments, the amount of the one or more additional pharmaceutically acceptable excipients in a liquid pharmaceutical formulation described herein can be about 3.5% (w/w).

In certain embodiments, the one or more additional pharmaceutically acceptable excipients is selected from the group consisting of ethanol, benzyl alcohol, glycerin, N-methyl-pyrrolidone (NMP), sodium chloride, sodium carbonate, mannitol, lactose, dextrose, a polyethylene glycol (PEG), propylene glycol, a polysorbate, a polyvinylpyrrolidone (PVP), a cyclodextrin or a derivative thereof, vitamin E or a derivative thereof, and combinations thereof.

In certain embodiments, the one or more additional pharmaceutically acceptable excipients comprises ethanol. In certain embodiments, the one or more additional pharmaceutically acceptable excipients comprises benzyl alcohol. In certain embodiments, the one or more additional pharmaceutically acceptable excipients comprises glycerin. In certain embodiments, the one or more additional pharmaceutically acceptable excipients comprises N-methyl-pyrrolidone (NMP). In certain embodiments, the one or more additional pharmaceutically acceptable excipients comprises sodium chloride. In certain embodiments, the one or more additional pharmaceutically acceptable excipients comprises sodium carbonate. In certain embodiments, the one or more additional pharmaceutically acceptable excipients comprises mannitol. In certain embodiments, the one or more additional pharmaceutically acceptable excipients comprises lactose. In certain embodiments, the one or more additional pharmaceutically acceptable excipients comprises dextrose. In certain embodiments, the one or more additional pharmaceutically acceptable excipients comprises a polyethylene glycol (PEG). In certain embodiments, the one or more additional pharmaceutically acceptable excipients comprises propylene glycol. In certain embodiments, the one or more additional pharmaceutically acceptable excipients comprises a polysorbate (e.g., polyoxyethylene (20) sorbitan monolaurate, polyoxyethylene (20) sorbitan monopalmitate, polyoxyethylene (20) sorbitan monostearate, polyoxyethylene (20) sorbitan monooleate). In certain embodiments, the one or more additional pharmaceutically acceptable excipients comprises a polyvinylpyrrolidone (PVP). In certain embodiments, the one or more additional pharmaceutically acceptable excipients comprises a cyclodextrin. In certain embodiments, the one or more additional pharmaceutically acceptable excipients comprises a cyclodextrin derivative. In certain embodiments, the one or more additional pharmaceutically acceptable excipients comprises vitamin E. In certain embodiments, the one or more additional pharmaceutically acceptable excipients comprises one or more naturally occurring forms of vitamin E (e.g., alpha-, beta-, gamma-, and delta-tocopherol and alpha-, beta-, gamma-, and delta-tocotrienol). In certain embodiments, the one or more additional pharmaceutically acceptable excipients comprises vitamin E derivative (e.g., tocopheryl acetate, tocopheryl glucoside, tocopheryl phosphate).

### (1) Benzyl Alcohol

In certain embodiments, a liquid pharmaceutical formulation described herein can further comprise benzyl alcohol. In certain embodiments, the benzyl alcohol in a liquid pharmaceutical formulation described herein may act as a solubilizing agent.

In certain embodiments, the amount of benzyl alcohol in a liquid pharmaceutical formulation described herein can be from about 0.1% (w/w) to about 10% (w/w), from about 0.5% (w/w) to about 10% (w/w), from about 1% (w/w) to about 10% (w/w), from about 1.5% (w/w) to about 10% (w/w), from about 2% (w/w) to about 10% (w/w), from about 2.5% (w/w) to about 10% (w/w), from about 3% (w/w) to about 10% (w/w), from about 3.5% (w/w) to about 10% (w/w), from about 4% (w/w) to about 10% (w/w), from about 4.5% (w/w) to about 10% (w/w), from about 5% (w/w) to about 10% (w/w), from about 6% (w/w) to about 10% (w/w), from about 7% (w/w) to about 10% (w/w), from about 8% (w/w) to about 10% (w/w), from about 9% (w/w) to about 10% (w/w), from about 0.1% (w/w) to about 9% (w/w), from about 0.1% (w/w) to about 8% (w/w), from about 0.1% (w/w) to about 7% (w/w), from about 0.1% (w/w) to about 6% (w/w), from about 0.1% (w/w) to about 4.5% (w/w), from about 0.1% (w/w) to about 4% (w/w), from about 0.1% (w/w) to about 3.5% (w/w), from about 0.1% (w/w) to about 3% (w/w), from about 0.1% (w/w) to about 2.5% (w/w), from about 0.1% (w/w) to about 2% (w/w), from about 0.1% (w/w) to about 1.5% (w/w), from about 0.1% (w/w) to about 1% (w/w), from about 0.1% (w/w) to about 0.5% (w/w), from about 0.5% (w/w) to about 9% (w/w), from about 0.5% (w/w) to about 8% (w/w), from about 0.5% (w/w) to about 7% (w/w), from about 0.5% (w/w) to about 6% (w/w), from about 0.5% (w/w) to about 5% (w/w), from about 0.5% (w/w) to about 4.5% (w/w), from about 0.5% (w/w) to about 4% (w/w), from about 0.5% (w/w) to about 3.5% (w/w), from about 0.5% (w/w) to about 3% (w/w), from about 0.5% (w/w) to about 2.5% (w/w), from about 0.5% (w/w) to about 2% (w/w), from about 0.5% (w/w) to about 1.5% (w/w), from about 0.5% (w/w) to about 1% (w/w), from about 1% (w/w) to about 9% (w/w), from about 1% (w/w) to about 8% (w/w), from about 1% (w/w) to about 7% (w/w), from about 1% (w/w) to about 6% (w/w), from about 1% (w/w) to about 5% (w/w), from about 1% (w/w) to about 4.5% (w/w), from about 1% (w/w) to about 4% (w/w), from about 1% (w/w) to about 3.5% (w/w), from about 1% (w/w) to about 3% (w/w), from about 1% (w/w) to about 2.5% (w/w), from about 1% (w/w) to about 2% (w/w), from about 1% (w/w) to about 1.5% (w/w), from about 1.5% (w/w) to about 9% (w/w), from about 1.5% (w/w) to about 8% (w/w), from about 1.5% (w/w) to about 7% (w/w), from about 1.5% (w/w) to about 6% (w/w), from about 1.5% (w/w) to about 5% (w/w), from about 1.5% (w/w) to about 4.5% (w/w), from about 1.5% (w/w) to about 4% (w/w), from about 1.5% (w/w) to about 3.5% (w/w), from about 1.5% (w/w) to about 3% (w/w), from about 1.5% (w/w) to about 2.5% (w/w), from about 1.5% (w/w) to about 2% (w/w), from about 2% (w/w) to about 9% (w/w), from about 2% (w/w) to about 8% (w/w), from about 2% (w/w) to about 7% (w/w), from about 2% (w/w) to about 6% (w/w), from about 2% (w/w) to about 5% (w/w), from about 2% (w/w) to about 4.5% (w/w), from about 2% (w/w) to about 4% (w/w), from about 2% (w/w) to about 3.5% (w/w), from about 2% (w/w) to about 3% (w/w), from about 2% (w/w) to about 2.5% (w/w), from about 2.5% (w/w) to about 9% (w/w), from about 2.5% (w/w) to about 8% (w/w), from about 2.5% (w/w) to about 7% (w/w), from about 2.5% (w/w) to about 6% (w/w), from about 2.5% (w/w) to about 5% (w/w), from about 2.5% (w/w) to about 4.5% (w/w), from about 2.5% (w/w) to about 4% (w/w), from about 2.5% (w/w) to about 3.5% (w/w), from about 2.5% (w/w) to about 3% (w/w), from about 3% (w/w) to about 9% (w/w), from about 3% (w/w) to about 8% (w/w), from about 3% (w/w) to about 7% (w/w), from about 3% (w/w) to about 6% (w/w), from about 3% (w/w) to about 5% (w/w), from about 3% (w/w) to about 4.5% (w/w), from about 3% (w/w) to about 4% (w/w), from about 3% (w/w) to about 3.5% (w/w), from about 3.5% (w/w) to about 9% (w/w), from about 3.5% (w/w) to about 8% (w/w), from about 3.5% (w/w) to about 7% (w/w), from about 3.5% (w/w) to about 6% (w/w), from about 3.5% (w/w) to about 5% (w/w), from about 3.5% (w/w) to about 4.5% (w/w), from about 3.5% (w/w) to about 4% (w/w), from about 4% (w/w) to about 9% (w/w), from about 4% (w/w) to about 8% (w/w), from about 4% (w/w) to about 7% (w/w), from about 4% (w/w) to about 6% (w/w), from about 4% (w/w) to about 5% (w/w), from about 4% (w/w) to about 4.5% (w/w), from about 4.5% (w/w) to about 9% (w/w), from about 4.5% (w/w) to about 8% (w/w), from about 4.5% (w/w) to about 7% (w/w), from about 4.5% (w/w) to about 6% (w/w), from about 4.5% (w/w) to about 5% (w/w), from about 5% (w/w) to about 9% (w/w), from about 5% (w/w) to about 8% (w/w), from about 5% (w/w) to about 7% (w/w), from about 5% (w/w) to about 6% (w/w), from about 6% (w/w) to about 9% (w/w), from about 6% (w/w) to about 8% (w/w), from about 6% (w/w) to about 7% (w/w), from about 7% (w/w) to about 9% (w/w), from about 7% (w/w) to about 8% (w/w), or from about 8% (w/w) to about 9% (w/w). In certain embodiments, the amount of benzyl alcohol in a liquid pharmaceutical formulation described herein is from about 0.1% (w/w) to about 10% (w/w). In certain embodiments, the amount of benzyl alcohol in a liquid pharmaceutical formulation described herein is from about 1% (w/w) to about 3.5% (w/w).

In certain embodiments, the amount of benzyl alcohol in a liquid pharmaceutical formulation described herein can be about 0.1% (w/w), about 0.5% (w/w), about 1% (w/w), about 1.5% (w/w), about 2% (w/w), about 2.5% (w/w), about 3% (w/w), about 3.5% (w/w), about 4% (w/w), about 4.5% (w/w), about 5% (w/w), about 6% (w/w), about 7% (w/w), about 8% (w/w), about 9% (w/w), or about 10% (w/w). In certain embodiments, the amount of benzyl alcohol in a liquid pharmaceutical formulation described herein can be about 1% (w/w). In certain embodiments, the amount of benzyl alcohol in a liquid pharmaceutical formulation described herein can be about 3.5% (w/w).

### (2) Cyclodextrins and Derivatives

In certain embodiments, a liquid pharmaceutical formulation described herein may comprise a cyclodextrin. In certain embodiments, a liquid pharmaceutical formulation described herein may comprise a cyclodextrin derivative.

Cyclodextrins contemplated to be useful as pharmaceutically acceptable excipients include, but are not limited to, an α (alpha)-cyclodextrin, a β (beta)-cyclodextrin, and a (gamma)-cyclodextrin. In certain embodiments, a liquid pharmaceutical formulation described herein may comprise an α-cyclodextrin. In certain embodiments, a liquid pharmaceutical formulation described herein may comprise an β-cyclodextrin. In certain embodiments, a liquid pharmaceutical formulation described herein may comprise an γ-cyclodextrin.

Cyclodextrin derivatives contemplated to be useful as pharmaceutically acceptable excipients include, but are not limited to, a sulfobutyl-ether-β-cyclodextrin (e.g., captisol), a hydroxypropyl-β-cyclodextrin (e.g., 2-hydroxypropyl-β-cyclodextrin), and a methylated β-cyclodextrin (e.g., a randomly methylated β-cyclodextrin). In certain embodiments, a liquid pharmaceutical formulation described herein may comprise a sulfobutyl-ether-β-cyclodextrin. In certain embodiments, a liquid pharmaceutical formulation described herein may comprise Captisol®. In certain embodiments, a liquid pharmaceutical formulation described herein may comprise a hydroxypropyl-β-cyclodextrin. In certain embodiments, a liquid pharmaceutical formulation described herein may comprise a methylated β-cyclodextrin.

In certain embodiments, the amount of the cyclodextrin in the liquid pharmaceutical formulation is from about 5% to about 50% (w/w), from about 10% to about 50% (w/w), from about 15% to about 50% (w/w), from about 20% to about 50% (w/w), from about 20% to about 50% (w/w), from about 25% to about 50% (w/w), from about 30% to about 50% (w/w), from about 35% to about 50% (w/w), from about 40% to about 50% (w/w), from about 45% to about 50% (w/w), from about 5% to about 45% (w/w), from about 5% to about 40% (w/w), from about 5% to about 35% (w/w), from about 5% to about 30% (w/w), from about 5% to about 25% (w/w), from about 5% to about 20% (w/w), from about 5% to about 15% (w/w), from about 5% to about 10% (w/w), from about 10% to about 45% (w/w), from about 10% to about 40% (w/w), from about 10% to about 35% (w/w), from about 10% to about 30% (w/w), from about 10% to about 25% (w/w), from about 10% to about 20% (w/w), from about 10% to about 15% (w/w), from about 15% to about 45% (w/w), from about 15% to about 40% (w/w), from about 15% to about 35% (w/w), from about 15% to about 30% (w/w), from about 15% to about 25% (w/w), from about 15% to about 20% (w/w), from about 20% to about 45% (w/w), from about 20% to about 40% (w/w), from about 20% to about 35% (w/w), from about 20% to about 30% (w/w), from about 20% to about 25% (w/w), from about 25% to about 45% (w/w), from about 25% to about 40% (w/w), from about 25% to about 35% (w/w), from about 25% to about 30% (w/w), from about 30% to about 45% (w/w), from about 30% to about 40% (w/w), from about 30% to about 35% (w/w), from about 35% to about 45% (w/w), from about 35% to about 40% (w/w), or from about 40% to about 45% (w/w).

In certain embodiments, the amount of the cyclodextrin derivative in the liquid pharmaceutical formulation is from about 5% to about 50% (w/w), from about 10% to about 50% (w/w), from about 15% to about 50% (w/w), from about 20% to about 50% (w/w), from about 20% to about 50% (w/w), from about 25% to about 50% (w/w), from about 30% to about 50% (w/w), from about 35% to about 50% (w/w), from about 40% to about 50% (w/w), from about 45% to about 50% (w/w), from about 5% to about 45% (w/w), from about 5% to about 40% (w/w), from about 5% to about 35% (w/w), from about 5% to about 30% (w/w), from about 5% to about 25% (w/w), from about 5% to about 20% (w/w), from about 5% to about 15% (w/w), from about 5% to about 10% (w/w), from about 10% to about 45% (w/w), from about 10% to about 40% (w/w), from about 10% to about 35% (w/w), from about 10% to about 30% (w/w), from about 10% to about 25% (w/w), from about 10% to about 20% (w/w), from about 10% to about 15% (w/w), from about 15% to about 45% (w/w), from about 15% to about 40% (w/w), from about 15% to about 35% (w/w), from about 15% to about 30% (w/w), from about 15% to about 25% (w/w), from about 15% to about 20% (w/w), from about 20% to about 45% (w/w), from about 20% to about 40% (w/w), from about 20% to about 35% (w/w), from about 20% to about 30% (w/w), from about 20% to about 25% (w/w), from about 25% to about 45% (w/w), from about 25% to about 40% (w/w), from about 25% to about 35% (w/w), from about 25% to about 30% (w/w), from about 30% to about 45% (w/w), from about 30% to about 40% (w/w), from about 30% to about 35% (w/w), from about 35% to about 45% (w/w), from about 35% to about 40% (w/w), or from about 40% to about 45% (w/w).

In certain embodiments, the amount of the cyclodextrin in the liquid pharmaceutical formulation is less than or equal to 5% (w/w), less than or equal to 10% (w/w), less than or equal to 15% (w/w), less than or equal to 20% (w/w), less than or equal to 25% (w/w), less than or equal to 30% (w/w), less than or equal to 35% (w/w), less than or equal to 40% (w/w), less than or equal to 45% (w/w), or less than or equal to 50% (w/w).

In certain embodiments, the amount of the cyclodextrin derivative in the liquid pharmaceutical formulation is less than or equal to 5% (w/w), less than or equal to 10% (w/w), less than or equal to 15% (w/w), less than or equal to 20% (w/w), less than or equal to 25% (w/w), less than or equal to 30% (w/w), less than or equal to 35% (w/w), less than or equal to 40% (w/w), less than or equal to 45% (w/w), or less than or equal to 50% (w/w).

In certain embodiments, the amount of the cyclodextrin in the liquid pharmaceutical formulation is about 5% (w/w), about 6% (w/w), about 7% (w/w), about 8% (w/w), about 9% (w/w), about 10% (w/w), about 11% (w/w), about 12% (w/w), about 13% (w/w), about 14% (w/w), about 15% (w/w), about 16% (w/w), about 17% (w/w), about 18% (w/w), about 19% (w/w), about 20% (w/w), about 25% (w/w), about 30% (w/w), about 35% (w/w), about 40% (w/w), about 45% (w/w), or about 50% (w/w).

In certain embodiments, the amount of the cyclodextrin derivative in the liquid pharmaceutical formulation is about 5% (w/w), about 6% (w/w), about 7% (w/w), about 8% (w/w), about 9% (w/w), about 10% (w/w), about 11% (w/w), about 12% (w/w), about 13% (w/w), about 14% (w/w), about 15% (w/w), about 16% (w/w), about 17% (w/w), about 18% (w/w), about 19% (w/w), about 20% (w/w), about 25% (w/w), about 30% (w/w), about 35% (w/w), about 40% (w/w), about 45% (w/w), or about 50% (w/w).

In certain embodiments, the concentration of the cyclodextrin in the liquid pharmaceutical formulation is from about 40 mM to about 200 mM, from about 50 mM to about 200 mM, from about 60 mM to about 200 mM, from about 70 mM to about 200 mM, from about 80 mM to about 200 mM, from about 90 mM to about 200 mM, from about 100 mM to about 200 mM, from about 120 mM to about 200 mM, from about 140 mM to about 200 mM, from about 160 mM to about 200 mM, from about 180 mM to about 200 mM, from about 40 mM to about 180 mM, from about 40 mM to about 160 mM, from about 40 mM to about 140 mM, from about 40 mM to about 120 mM, from about 40 mM to about 100 mM, from about 40 mM to about 90 mM, from about 40 mM to about 80 mM, from about 40 mM to about 70 mM, from about 40 mM to about 60 mM, from about 40 mM to about 50 mM, from about 50 mM to about 180 mM, from about 50 mM to about 160 mM, from about 50 mM to about 140 mM, from about 50 mM to about 120 mM, from about 50 mM to about 100 mM, from about 50 mM to about 90 mM, from about 50 mM to about 80 mM, from about 50 mM to about 70 mM, from about 50 mM to about 60 mM, from about 60 mM to about 180 mM, from about 60 mM to about 160 mM, from about 60 mM to about 140 mM, from about 60 mM to about 120 mM, from about 60 mM to about 100 mM, from about 60 mM to about 90 mM, from about 60 mM to about 80 mM, from about 60 mM to about 70 mM, from about 70 mM to about 180 mM, from about 70 mM to about 160 mM, from about 70 mM to about 140 mM, from about 70 mM to about 120 mM, from about 70 mM to about 100 mM, from about 70 mM to about 90 mM, from about 70 mM to about 80 mM, from about 80 mM to about 180 mM, from about 80 mM to about 160 mM, from about 80 mM to about 140 mM, from about 80 mM to about 120 mM, from about 80 mM to about 100 mM, from about 80 mM to about 90 mM, from about 90 mM to about 180 mM, from about 90 mM to about 160 mM, from about 90 mM to about 140 mM, from about 90 mM to about 120 mM, from about 90 mM to about 100 mM, from about 100 mM to about 180 mM, from about 100 mM to about 160 mM, from about 100 mM to about 140 mM, from about 100 mM to about 120 mM, from about 120 mM to about 180 mM, from about 120 mM to about 160 mM, from about 120 mM to about 140 mM, from about 140 mM to about 180 mM, from about 140 mM to about 160 mM, or from about 160 mM to about 180 mM.

In certain embodiments, the concentration of the cyclodextrin derivative in the liquid pharmaceutical formulation is from about 40 mM to about 200 mM, from about 50 mM to about 200 mM, from about 60 mM to about 200 mM, from about 70 mM to about 200 mM, from about 80 mM to about 200 mM, from about 90 mM to about 200 mM, from about 100 mM to about 200 mM, from about 120 mM to about 200 mM, from about 140 mM to about 200 mM, from about 160 mM to about 200 mM, from about 180 mM to about 200 mM, from about 40 mM to about 180 mM, from about 40 mM to about 160 mM, from about 40 mM to about 140 mM, from about 40 mM to about 120 mM, from about 40 mM to about 100 mM, from about 40 mM to about 90 mM, from about 40 mM to about 80 mM, from about 40 mM to about 70 mM, from about 40 mM to about 60 mM, from about 40 mM to about 50 mM, from about 50 mM to about 180 mM, from about 50 mM to about 160 mM, from about 50 mM to about 140 mM, from about 50 mM to about 120 mM, from about 50 mM to about 100 mM, from about 50 mM to about 90 mM, from about 50 mM to about 80 mM, from about 50 mM to about 70 mM, from about 50 mM to about 60 mM, from about 60 mM to about 180 mM, from about 60 mM to about 160 mM, from about 60 mM to about 140 mM, from about 60 mM to about 120 mM, from about 60 mM to about 100 mM, from about 60 mM to about 90 mM, from about 60 mM to about 80 mM, from about 60 mM to about 70 mM, from about 70 mM to about 180 mM, from about 70 mM to about 160 mM, from about 70 mM to about 140 mM, from about 70 mM to about 120 mM, from about 70 mM to about 100 mM, from about 70 mM to about 90 mM, from about 70 mM to about 80 mM, from about 80 mM to about 180 mM, from about 80 mM to about 160 mM, from about 80 mM to about 140 mM, from about 80 mM to about 120 mM, from about 80 mM to about 100 mM, from about 80 mM to about 90 mM, from about 90 mM to about 180 mM, from about 90 mM to about 160 mM, from about 90 mM to about 140 mM, from about 90 mM to about 120 mM, from about 90 mM to about 100 mM, from about 100 mM to about 180 mM, from about 100 mM to about 160 mM, from about 100 mM to about 140 mM, from about 100 mM to about 120 mM, from about 120 mM to about 180 mM, from about 120 mM to about 160 mM, from about 120 mM to about 140 mM, from about 140 mM to about 180 mM, from about 140 mM to about 160 mM, or from about 160 mM to about 180 mM.

In certain embodiments, the concentration of the cyclodextrin in the liquid pharmaceutical formulation is about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 110 mM, about 120 mM, about 130 mM, about 140 mM, about 150 mM, about 160 mM, about 170 mM, about 180 mM, about 190 mM, or about 200 mM.

In certain embodiments, the concentration of the cyclodextrin derivative in the liquid pharmaceutical formulation is about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 110 mM, about 120 mM, about 130 mM, about 140 mM, about 150 mM, about 160 mM, about 170 mM, about 180 mM, about 190 mM, or about 200 mM.

In certain embodiments, the liquid pharmaceutical formulation further comprises water (e.g., water for injection (WFI)).

### Exemplary Furosemide Liquid Pharmaceutical Formulations

In various embodiments, provided herein are liquid pharmaceutical formulations for treating a disease or disorder described herein (e.g., congestion, edema, fluid overload, or hypertension) in a patient in need thereof, which comprise a therapeutically effective amount of furosemide, and a pharmaceutically acceptable excipient. The liquid pharmaceutical formulation can include, among other things, a pharmaceutically acceptable pH adjuster, an antioxidant, an osmolarity adjuster, and/or a pharmaceutically acceptable buffer as described herein.

In various embodiments, a liquid pharmaceutical formulation comprises:
(i) about 1 mg/mL to about 250 mg/mL furosemide, or a pharmaceutically acceptable salt thereof; and
(ii) about 10 mM to about 500 mM of a pharmaceutically acceptable buffer.

In various embodiments, a liquid pharmaceutical formulation comprises:
(i) furosemide, or a pharmaceutically acceptable salt thereof, in a therapeutically effective amount; and
(ii) tromethamine, or a pharmaceutically acceptable salt thereof.

In various embodiments, a liquid pharmaceutical formulation comprises:
(i) about 1 mg/mL to about 250 mg/mL furosemide, or a pharmaceutically acceptable salt thereof; and
(ii) about 10 mM to about 250 mM tromethamine, or a pharmaceutically acceptable salt thereof.

In various embodiments, a liquid pharmaceutical formulation comprises:
(i) furosemide, or a pharmaceutically acceptable salt thereof, in a therapeutically effective amount;
(ii) a pharmaceutically acceptable buffer; and
(iii) a pharmaceutically acceptable pH adjuster.

In various embodiments, a liquid pharmaceutical formulation comprises:
(i) furosemide, or a pharmaceutically acceptable salt thereof, in a therapeutically effective amount;
(ii) a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) a pharmaceutically acceptable pH adjuster selected from the group consisting of acetic acid, citric acid, fumaric acid, hydrochloric acid, malic acid, nitric acid, phosphoric acid, propionic acid, sulfuric acid, tartaric acid, ammonia solution, ammonium carbonate, diethanolamine, potassium hydroxide, sodium bicarbonate, sodium borate, sodium carbonate, sodium hydroxide, trolamine, and combinations thereof.

In various embodiments, a liquid pharmaceutical formulation comprises:
(i) about 1 mg/mL to about 250 mg/mL furosemide, or a pharmaceutically acceptable salt thereof;
(ii) about 10 mM to about 500 mM of a pharmaceutically acceptable buffer; and
(iii) a pharmaceutically acceptable pH adjuster.

In various embodiments, a liquid pharmaceutical formulation comprises:
(i) about 1 mg/mL to about 250 mg/mL furosemide, or a pharmaceutically acceptable salt thereof;
(ii) about 10 mM to about 500 mM of a pharmaceutically acceptable buffer; and
(iii) a pharmaceutically acceptable pH adjuster.

In various embodiments, a liquid pharmaceutical formulation comprises:
(i) about 1 mg/mL to about 250 mg/mL furosemide, or a pharmaceutically acceptable salt thereof;
(ii) about 10 mM to about 500 mM of a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof, and
(iii) a pharmaceutically acceptable pH adjuster selected from the group consisting of acetic acid, citric acid, fumaric acid, hydrochloric acid, malic acid, nitric acid, phosphoric acid, propionic acid, sulfuric acid, tartaric acid, ammonia solution, ammonium carbonate, diethanolamine, potassium hydroxide, sodium bicarbonate, sodium borate, sodium carbonate, sodium hydroxide, trolamine, and combinations thereof.

In various embodiments, a liquid pharmaceutical formulation comprises:
(i) about 1 mg/mL to about 250 mg/mL furosemide, or a pharmaceutically acceptable salt thereof;
(ii) about 10 mM to about 250 mM tromethamine, or a pharmaceutically acceptable salt thereof; and
(iii) a pharmaceutically acceptable pH adjuster comprising sodium hydroxide and hydrochloric acid.

In various embodiments, a liquid pharmaceutical formulation comprises:
(i) furosemide, or a pharmaceutically acceptable salt thereof, in a therapeutically effective amount;
(ii) a pharmaceutically acceptable buffer; and
(iii) an antioxidant.

In various embodiments, a liquid pharmaceutical formulation comprises:
(i) furosemide, or a pharmaceutically acceptable salt thereof, in a therapeutically effective amount;
(ii) a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) an antioxidant selected from the group consisting of sodium metabisulfite, sodium bisulfite monothioglycerol, methionine, ethylenediaminetetraacetic acid (EDTA), and combinations thereof.

In various embodiments, a liquid pharmaceutical formulation comprises:
(i) about 1 mg/mL to about 250 mg/mL furosemide, or a pharmaceutically acceptable salt thereof;
(ii) about 10 mM to about 500 mM of a pharmaceutically acceptable buffer; and (iii) about 0.01% (w/w) to about 2% (w/w) of an antioxidant.

In various embodiments, a liquid pharmaceutical formulation comprises:
(i) about 1 mg/mL to about 250 mg/mL furosemide, or a pharmaceutically acceptable salt thereof;
(ii) about 10 mM to about 500 mM of a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) about 0.01% (w/w) to about 2% (w/w) of an antioxidant selected from the group consisting of sodium metabisulfite, sodium bisulfite monothioglycerol, methionine, ethylenediaminetetraacetic acid (EDTA), and combinations thereof.

In various embodiments, a liquid pharmaceutical formulation comprises:
(i) about 1 mg/mL to about 250 mg/mL furosemide, or a pharmaceutically acceptable salt thereof;
(ii) about 10 mM to about 250 mM tromethamine, or a pharmaceutically acceptable salt thereof; and
(iii) about 0.01% (w/w) to about 2% (w/w) sodium metabisulfite.

In various embodiments, a liquid pharmaceutical formulation comprises:
(i) furosemide, or a pharmaceutically acceptable salt thereof, in a therapeutically effective amount;
(ii) a pharmaceutically acceptable buffer; and
(iii) an osmolarity adjuster.

In various embodiments, a liquid pharmaceutical formulation comprises:
(i) furosemide, or a pharmaceutically acceptable salt thereof, in a therapeutically effective amount;
(ii) a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) an osmolarity adjuster selected from the group consisting of sodium chloride, potassium chloride, isosorbide, mannitol, xylitol, and combinations thereof.

In various embodiments, a liquid pharmaceutical formulation comprises:
(i) about 1 mg/mL to about 250 mg/mL furosemide, or a pharmaceutically acceptable salt thereof;
(ii) about 10 mM to about 500 mM of a pharmaceutically acceptable buffer; and
(iii) an osmolarity adjuster.

In various embodiments, a liquid pharmaceutical formulation comprises:
(i) about 1 mg/mL to about 250 mg/mL furosemide, or a pharmaceutically acceptable salt thereof;
(ii) about 10 mM to about 500 mM of a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof, and
(iii) an osmolarity adjuster selected from the group consisting of sodium chloride, potassium chloride, isosorbide, mannitol, xylitol, and combinations thereof.

In various embodiments, a liquid pharmaceutical formulation comprises:
(i) about 1 mg/mL to about 250 mg/mL furosemide, or a pharmaceutically acceptable salt thereof;
(ii) about 10 mM to about 250 mM tromethamine, or a pharmaceutically acceptable salt thereof; and
(iii) sodium chloride.

In various embodiments, a liquid pharmaceutical formulation comprises:
(i) furosemide, or a pharmaceutically acceptable salt thereof, in a therapeutically effective amount;
(ii) a pharmaceutically acceptable buffer; and
(iii) one or more additional pharmaceutically acceptable excipients.

In various embodiments, a liquid pharmaceutical formulation comprises:
(i) furosemide, or a pharmaceutically acceptable salt thereof, in a therapeutically effective amount;
(ii) a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) one or more additional pharmaceutically acceptable excipients selected from the group consisting of ethanol, benzyl alcohol, glycerin, N-methyl-pyrrolidone (NMP), sodium chloride, sodium carbonate, mannitol, lactose, dextrose, a polyethylene glycol (PEG), propylene glycol, a polysorbate, a polyvinylpyrrolidone (PVP), a cyclodextrin or a derivative thereof, vitamin E or a derivative thereof, and combinations thereof.

In various embodiments, a liquid pharmaceutical formulation comprises:
(i) about 1 mg/mL to about 250 mg/mL furosemide, or a pharmaceutically acceptable salt thereof;
(ii) about 10 mM to about 500 mM of a pharmaceutically acceptable buffer; and
(iii) about 0.1% (w/w) to about 30% (w/w) one or more additional pharmaceutically acceptable excipients.

In various embodiments, a liquid pharmaceutical formulation comprises:
(i) about 1 mg/mL to about 250 mg/mL furosemide, or a pharmaceutically acceptable salt thereof;
(ii) about 10 mM to about 500 mM of a pharmaceutically acceptable buffer comprising a buffering agent selected from the group consisting of histidine, a citrate salt, sodium phosphate, potassium phosphate, tromethamine or a pharmaceutically acceptable salt thereof, and
(iii) about 0.1% (w/w) to about 30% (w/w) one or more additional pharmaceutically acceptable excipients selected from the group consisting of ethanol, benzyl alcohol, glycerin, N-methyl-pyrrolidone (NMP), sodium chloride, sodium carbonate, mannitol, lactose, dextrose, a polyethylene glycol (PEG), propylene glycol, a polysorbate, a polyvinylpyrrolidone (PVP), a cyclodextrin or a derivative thereof, vitamin E or a derivative thereof, and combinations thereof.

In various embodiments, a liquid pharmaceutical formulation comprises:
(i) about 1 mg/mL to about 250 mg/mL furosemide, or a pharmaceutically acceptable salt thereof;
(ii) about 10 mM to about 250 mM tromethamine, or a pharmaceutically acceptable salt thereof; and
(iii) about 0.1% (w/w) to about 30% (w/w) one or more additional pharmaceutically acceptable excipients.

In various embodiments, a liquid pharmaceutical formulation comprises:
(i) about 1 mg/mL to about 250 mg/mL furosemide, or a pharmaceutically acceptable salt thereof;
(ii) about 10 mM to about 250 mM tromethamine, or a pharmaceutically acceptable salt thereof; and
(iii) about 0.1% (w/w) to about 30% (w/w) one or more additional pharmaceutically acceptable excipients selected from the group consisting of ethanol, benzyl alcohol, glycerin, N-methyl-pyrrolidone (NMP), sodium chloride, sodium carbonate, mannitol, lactose, dextrose, a polyethylene glycol (PEG), propylene glycol, a polysorbate, a polyvinylpyrrolidone (PVP), a cyclodextrin or a derivative thereof, vitamin E or a derivative thereof, and combinations thereof.

In various embodiments, a liquid pharmaceutical formulation comprises:
(i) furosemide, or a pharmaceutically acceptable salt thereof, in a therapeutically effective amount; and
(ii) a cyclodextrin or a derivative thereof.

In various embodiments, a liquid pharmaceutical formulation comprises:
(i) about 1 mg/mL to about 250 mg/mL furosemide, or a pharmaceutically acceptable salt thereof; and
(ii) about 5% to about 50% (w/w) of a cyclodextrin or a derivative thereof.

In various embodiments, a liquid pharmaceutical formulation comprises:
(i) furosemide, or a pharmaceutically acceptable salt thereof, in a therapeutically effective amount;
(ii) a pharmaceutically acceptable buffer; and
(iii) a cyclodextrin or a derivative thereof.

In various embodiments, a liquid pharmaceutical formulation comprises:
(i) about 1 mg/mL to about 250 mg/mL furosemide, or a pharmaceutically acceptable salt thereof;
(ii) about 10 mM to about 500 mM of a pharmaceutically acceptable buffer; and
(iii) about 5% to about 50% (w/w) of a cyclodextrin or a derivative thereof.

In various embodiments, a liquid pharmaceutical formulation comprises:
(i) furosemide, or a pharmaceutically acceptable salt thereof, in a therapeutically effective amount;
(ii) tromethamine, or a pharmaceutically acceptable salt thereof; and
(iii) a cyclodextrin or a derivative thereof.

In various embodiments, a liquid pharmaceutical formulation comprises:
(i) about 1 mg/mL to about 250 mg/mL furosemide, or a pharmaceutically acceptable salt thereof;
(ii) about 10 mM to about 250 mM tromethamine, or a pharmaceutically acceptable salt thereof; and
(iii) about 5% to about 50% (w/w) of a cyclodextrin or a derivative thereof.

In various embodiments, a liquid pharmaceutical formulation comprises:
(i) furosemide, or a pharmaceutically acceptable salt thereof, in a therapeutically effective amount; and
(ii) benzyl alcohol.

In various embodiments, a liquid pharmaceutical formulation comprises:
(i) about 1 mg/mL to about 250 mg/mL furosemide, or a pharmaceutically acceptable salt thereof; and
(ii) about 0.1% (w/w) to about 10% (w/w) benzyl alcohol.

In various embodiments, a liquid pharmaceutical formulation comprises:
(i) furosemide, or a pharmaceutically acceptable salt thereof, in a therapeutically effective amount;
(ii) a pharmaceutically acceptable buffer; and
(iii) benzyl alcohol

In various embodiments, a liquid pharmaceutical formulation comprises:
(i) about 1 mg/mL to about 250 mg/mL furosemide, or a pharmaceutically acceptable salt thereof;
(ii) about 10 mM to about 500 mM of a pharmaceutically acceptable buffer; and
(iii) about 0.1% (w/w) to about 10% (w/w) benzyl alcohol.

In various embodiments, a liquid pharmaceutical formulation comprises:
(i) furosemide, or a pharmaceutically acceptable salt thereof, in a therapeutically effective amount;
(ii) tromethamine, or a pharmaceutically acceptable salt thereof; and
(iii) benzyl alcohol.

In various embodiments, a liquid pharmaceutical formulation comprises:
(i) about 1 mg/mL to about 250 mg/mL furosemide, or a pharmaceutically acceptable salt thereof;
(ii) about 10 mM to about 250 mM tromethamine, or a pharmaceutically acceptable salt thereof; and
(iii) about 0.1% (w/w) to about 10% (w/w) benzyl alcohol.

In various embodiments, a liquid pharmaceutical formulation comprises:
(i) furosemide, or a pharmaceutically acceptable salt thereof, in a therapeutically effective amount;
(ii) tromethamine, or a pharmaceutically acceptable salt thereof;
(iii) benzyl alcohol;
(iv) sodium chloride; and
(v) a pH adjuster comprising sodium hydroxide and/or hydrochloric acid.

In various embodiments, a liquid pharmaceutical formulation comprises:
(i) about 1 mg/mL to about 250 mg/mL furosemide, or a pharmaceutically acceptable salt thereof;
(ii) about 10 mM to about 250 mM tromethamine, or a pharmaceutically acceptable salt thereof;
(iii) about 0.1% (w/w) to about 10% (w/w) benzyl alcohol;
(iv) sodium chloride; and
(v) a pH adjuster comprising sodium hydroxide and/or hydrochloric acid.

In various embodiments, a liquid pharmaceutical formulation comprises:
(i) about 1 mg/mL to about 250 mg/mL furosemide, or a pharmaceutically acceptable salt thereof;
(ii) about 10 mM to about 250 mM tromethamine, or a pharmaceutically acceptable salt thereof; and
(iii) about 0.01% (w/w) to about 2% (w/w) sodium metabisulfite.

### Exemplary Bumetanide Liquid Pharmaceutical Formulations

In various embodiments, provided herein are liquid pharmaceutical formulations for treating a disease or disorder described herein (e.g., congestion, edema, fluid overload, or hypertension) in a patient in need thereof, which comprise a therapeutically effective amount of bumetanide, and a pharmaceutically acceptable excipient. The liquid pharmaceutical formulation can include, among other things, a pharmaceutically acceptable pH adjuster, an antioxidant, an osmolarity adjuster, and/or a pharmaceutically acceptable buffer as described herein.

In various embodiments, a liquid pharmaceutical formulation comprises:
(i) about 0.025 mg/mL to about 65 mg/mL bumetanide, or a pharmaceutically acceptable salt thereof; and
(ii) about 10 mM to about 500 mM of a pharmaceutically acceptable buffer.

In various embodiments, a liquid pharmaceutical formulation comprises:
(i) bumetanide, or a pharmaceutically acceptable salt thereof, in a therapeutically effective amount; and
(ii) tromethamine, or a pharmaceutically acceptable salt thereof.

In various embodiments, a liquid pharmaceutical formulation comprises:
(i) about 0.025 mg/mL to about 65 mg/mL bumetanide, or a pharmaceutically acceptable salt thereof; and
(ii) about 10 mM to about 250 mM tromethamine, or a pharmaceutically acceptable salt thereof.

In various embodiments, a liquid pharmaceutical formulation comprises:
(i) bumetanide, or a pharmaceutically acceptable salt thereof, in a therapeutically effective amount;
(ii) a pharmaceutically acceptable buffer; and
(iii) a pharmaceutically acceptable pH adjuster.

In various embodiments, a liquid pharmaceutical formulation comprises:
(i) bumetanide, or a pharmaceutically acceptable salt thereof, in a therapeutically effective amount;
(ii) a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) a pharmaceutically acceptable pH adjuster selected from the group consisting of acetic acid, citric acid, fumaric acid, hydrochloric acid, malic acid, nitric acid, phosphoric acid, propionic acid, sulfuric acid, tartaric acid, ammonia solution, ammonium carbonate, diethanolamine, potassium hydroxide, sodium bicarbonate, sodium borate, sodium carbonate, sodium hydroxide, trolamine, and combinations thereof.

In various embodiments, a liquid pharmaceutical formulation comprises:
(i) about 0.025 mg/mL to about 65 mg/mL bumetanide, or a pharmaceutically acceptable salt thereof;
(ii) about 10 mM to about 500 mM of a pharmaceutically acceptable buffer; and
(iii) a pharmaceutically acceptable pH adjuster.

In various embodiments, a liquid pharmaceutical formulation comprises:
(i) about 0.025 mg/mL to about 65 mg/mL bumetanide, or a pharmaceutically acceptable salt thereof;
(ii) about 10 mM to about 500 mM of a pharmaceutically acceptable buffer; and (iii) a pharmaceutically acceptable pH adjuster.

In various embodiments, a liquid pharmaceutical formulation comprises:
(i) about 0.025 mg/mL to about 65 mg/mL bumetanide, or a pharmaceutically acceptable salt thereof;
(ii) about 10 mM to about 500 mM of a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof, and
(iii) a pharmaceutically acceptable pH adjuster selected from the group consisting of acetic acid, citric acid, fumaric acid, hydrochloric acid, malic acid, nitric acid, phosphoric acid, propionic acid, sulfuric acid, tartaric acid, ammonia solution, ammonium carbonate, diethanolamine, potassium hydroxide, sodium bicarbonate, sodium borate, sodium carbonate, sodium hydroxide, trolamine, and combinations thereof.

In various embodiments, a liquid pharmaceutical formulation comprises:
(i) about 0.025 mg/mL to about 65 mg/mL bumetanide, or a pharmaceutically acceptable salt thereof;
(ii) about 10 mM to about 250 mM tromethamine, or a pharmaceutically acceptable salt thereof; and
(iii) a pharmaceutically acceptable pH adjuster comprising sodium hydroxide and hydrochloric acid.

In various embodiments, a liquid pharmaceutical formulation comprises:
(i) bumetanide, or a pharmaceutically acceptable salt thereof, in a therapeutically effective amount;
(ii) a pharmaceutically acceptable buffer; and
(iii) an antioxidant.

In various embodiments, a liquid pharmaceutical formulation comprises:
(i) bumetanide, or a pharmaceutically acceptable salt thereof, in a therapeutically effective amount;
(ii) a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) an antioxidant selected from the group consisting of sodium metabisulfite, sodium bisulfite monothioglycerol, methionine, ethylenediaminetetraacetic acid (EDTA), and combinations thereof.

In various embodiments, a liquid pharmaceutical formulation comprises:
(i) about 0.025 mg/mL to about 65 mg/mL bumetanide, or a pharmaceutically acceptable salt thereof;
(ii) about 10 mM to about 500 mM of a pharmaceutically acceptable buffer; and
(iii) about 0.01% (w/w) to about 2% (w/w) of an antioxidant.

In various embodiments, a liquid pharmaceutical formulation comprises:
(i) about 0.025 mg/mL to about 65 mg/mL bumetanide, or a pharmaceutically acceptable salt thereof;
(ii) about 10 mM to about 500 mM of a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) about 0.01% (w/w) to about 2% (w/w) of an antioxidant selected from the group consisting of sodium metabisulfite, sodium bisulfite monothioglycerol, methionine, ethylenediaminetetraacetic acid (EDTA), and combinations thereof.

In various embodiments, a liquid pharmaceutical formulation comprises:
(i) about 0.025 mg/mL to about 65 mg/mL bumetanide, or a pharmaceutically acceptable salt thereof;
(ii) about 10 mM to about 250 mM tromethamine, or a pharmaceutically acceptable salt thereof; and
(iii) about 0.01% (w/w) to about 2% (w/w) sodium metabisulfite.

In various embodiments, a liquid pharmaceutical formulation comprises:
(i) bumetanide, or a pharmaceutically acceptable salt thereof, in a therapeutically effective amount;
(ii) a pharmaceutically acceptable buffer; and
(iii) an osmolarity adjuster.

In various embodiments, a liquid pharmaceutical formulation comprises:
(i) bumetanide, or a pharmaceutically acceptable salt thereof, in a therapeutically effective amount;
(ii) a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) an osmolarity adjuster selected from the group consisting of sodium chloride, potassium chloride, isosorbide, mannitol, xylitol, and combinations thereof.

In various embodiments, a liquid pharmaceutical formulation comprises:
(i) about 0.025 mg/mL to about 65 mg/mL bumetanide, or a pharmaceutically acceptable salt thereof;
(ii) about 10 mM to about 500 mM of a pharmaceutically acceptable buffer; and
(iii) an osmolarity adjuster.

In various embodiments, a liquid pharmaceutical formulation comprises:
(i) about 0.025 mg/mL to about 65 mg/mL bumetanide, or a pharmaceutically acceptable salt thereof;
(ii) about 10 mM to about 500 mM of a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) an osmolarity adjuster selected from the group consisting of sodium chloride, potassium chloride, isosorbide, mannitol, xylitol, and combinations thereof.

In various embodiments, a liquid pharmaceutical formulation comprises:
(i) about 0.025 mg/mL to about 65 mg/mL bumetanide, or a pharmaceutically acceptable salt thereof;
(ii) about 10 mM to about 250 mM tromethamine, or a pharmaceutically acceptable salt thereof; and
(iii) sodium chloride.

In various embodiments, a liquid pharmaceutical formulation comprises:
(i) bumetanide, or a pharmaceutically acceptable salt thereof, in a therapeutically effective amount;
(ii) a pharmaceutically acceptable buffer; and
(iii) one or more additional pharmaceutically acceptable excipients.

In various embodiments, a liquid pharmaceutical formulation comprises:
(i) bumetanide, or a pharmaceutically acceptable salt thereof, in a therapeutically effective amount;
(ii) a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) one or more additional pharmaceutically acceptable excipients selected from the group consisting of ethanol, benzyl alcohol, glycerin, N-methyl-pyrrolidone (NMP), sodium chloride, sodium carbonate, mannitol, lactose, dextrose, a polyethylene glycol (PEG), propylene glycol, a polysorbate, a polyvinylpyrrolidone (PVP), a cyclodextrin or a derivative thereof, vitamin E or a derivative thereof, and combinations thereof.

In various embodiments, a liquid pharmaceutical formulation comprises:
(i) about 0.025 mg/mL to about 65 mg/mL bumetanide, or a pharmaceutically acceptable salt thereof;
(ii) about 10 mM to about 500 mM of a pharmaceutically acceptable buffer; and
(iii) about 0.1% (w/w) to about 30% (w/w) one or more additional pharmaceutically acceptable excipients.

In various embodiments, a liquid pharmaceutical formulation comprises:
(i) about 0.025 mg/mL to about 65 mg/mL bumetanide, or a pharmaceutically acceptable salt thereof;
(ii) about 10 mM to about 500 mM of a pharmaceutically acceptable buffer comprising a buffering agent selected from the group consisting of histidine, a citrate salt, sodium phosphate, potassium phosphate, tromethamine or a pharmaceutically acceptable salt thereof; and
(iii) about 0.1% (w/w) to about 30% (w/w) one or more additional pharmaceutically acceptable excipients selected from the group consisting of ethanol, benzyl alcohol, glycerin, N-methyl-pyrrolidone (NMP), sodium chloride, sodium carbonate, mannitol, lactose, dextrose, a polyethylene glycol (PEG), propylene glycol, a polysorbate, a polyvinylpyrrolidone (PVP), a cyclodextrin or a derivative thereof, vitamin E or a derivative thereof, and combinations thereof.

In various embodiments, a liquid pharmaceutical formulation comprises:
(i) about 0.025 mg/mL to about 65 mg/mL bumetanide, or a pharmaceutically acceptable salt thereof;
(ii) about 10 mM to about 250 mM tromethamine, or a pharmaceutically acceptable salt thereof; and
(iii) about 0.1% (w/w) to about 30% (w/w) one or more additional pharmaceutically acceptable excipients.

In various embodiments, a liquid pharmaceutical formulation comprises:
(i) about 0.025 mg/mL to about 65 mg/mL bumetanide, or a pharmaceutically acceptable salt thereof;
(ii) about 10 mM to about 250 mM tromethamine, or a pharmaceutically acceptable salt thereof; and
(iii) about 0.1% (w/w) to about 30% (w/w) one or more additional pharmaceutically acceptable excipients selected from the group consisting of ethanol, benzyl alcohol, glycerin, N-methyl-pyrrolidone (NMP), sodium chloride, sodium carbonate, mannitol, lactose, dextrose, a polyethylene glycol (PEG), propylene glycol, a polysorbate, a polyvinylpyrrolidone (PVP), a cyclodextrin or a derivative thereof, vitamin E or a derivative thereof, and combinations thereof.

In various embodiments, a liquid pharmaceutical formulation comprises:
(i) bumetanide, or a pharmaceutically acceptable salt thereof, in a therapeutically effective amount; and
(ii) a cyclodextrin or a derivative thereof.

In various embodiments, a liquid pharmaceutical formulation comprises:
(i) about 0.025 mg/mL to about 65 mg/mL bumetanide, or a pharmaceutically acceptable salt thereof; and
(ii) about 5% to about 50% (w/w) of a cyclodextrin or a derivative thereof.

In various embodiments, a liquid pharmaceutical formulation comprises:
(i) bumetanide, or a pharmaceutically acceptable salt thereof, in a therapeutically effective amount;
(ii) a pharmaceutically acceptable buffer; and
(iii) a cyclodextrin or a derivative thereof.

In various embodiments, a liquid pharmaceutical formulation comprises:
(i) about 0.025 mg/mL to about 65 mg/mL bumetanide, or a pharmaceutically acceptable salt thereof;
(ii) about 10 mM to about 500 mM of a pharmaceutically acceptable buffer; and
(iii) about 5% to about 50% (w/w) of a cyclodextrin or a derivative thereof.

In various embodiments, a liquid pharmaceutical formulation comprises:
(i) bumetanide, or a pharmaceutically acceptable salt thereof, in a therapeutically effective amount;
(ii) tromethamine, or a pharmaceutically acceptable salt thereof; and
(iii) a cyclodextrin or a derivative thereof.

In various embodiments, a liquid pharmaceutical formulation comprises:
(i) about 0.025 mg/mL to about 65 mg/mL bumetanide, or a pharmaceutically acceptable salt thereof;
(ii) about 10 mM to about 250 mM tromethamine, or a pharmaceutically acceptable salt thereof; and
(iii) about 5% to about 50% (w/w) of a cyclodextrin or a derivative thereof.

In various embodiments, a liquid pharmaceutical formulation comprises:
(i) bumetanide, or a pharmaceutically acceptable salt thereof, in a therapeutically effective amount; and
(ii) benzyl alcohol.

In various embodiments, a liquid pharmaceutical formulation comprises:
(i) about 0.025 mg/mL to about 65 mg/mL bumetanide, or a pharmaceutically acceptable salt thereof; and
(ii) about 0.1% (w/w) to about 10% (w/w) benzyl alcohol.

In various embodiments, a liquid pharmaceutical formulation comprises:
(i) bumetanide, or a pharmaceutically acceptable salt thereof, in a therapeutically effective amount;
(ii) a pharmaceutically acceptable buffer; and
(iii) benzyl alcohol

In various embodiments, a liquid pharmaceutical formulation comprises:
(i) about 0.025 mg/mL to about 65 mg/mL bumetanide, or a pharmaceutically acceptable salt thereof;
(ii) about 10 mM to about 500 mM of a pharmaceutically acceptable buffer; and
(iii) about 0.1% (w/w) to about 10% (w/w) benzyl alcohol.

In various embodiments, a liquid pharmaceutical formulation comprises:
(i) bumetanide, or a pharmaceutically acceptable salt thereof, in a therapeutically effective amount;
(ii) tromethamine, or a pharmaceutically acceptable salt thereof; and
(iii) benzyl alcohol.

In various embodiments, a liquid pharmaceutical formulation comprises:
(i) about 0.025 mg/mL to about 65 mg/mL bumetanide, or a pharmaceutically acceptable salt thereof;
(ii) about 10 mM to about 250 mM tromethamine, or a pharmaceutically acceptable salt thereof; and
(iii) about 0.1% (w/w) to about 10% (w/w) benzyl alcohol.

In various embodiments, a liquid pharmaceutical formulation comprises:
(i) bumetanide, or a pharmaceutically acceptable salt thereof, in a therapeutically effective amount;
(ii) tromethamine, or a pharmaceutically acceptable salt thereof;
(iii) benzyl alcohol;
(iv) sodium chloride; and
(v) a pH adjuster comprising sodium hydroxide and/or hydrochloric acid.

In various embodiments, a liquid pharmaceutical formulation comprises:
(i) about 0.025 mg/mL to about 65 mg/mL bumetanide, or a pharmaceutically acceptable salt thereof;
(ii) about 10 mM to about 250 mM tromethamine, or a pharmaceutically acceptable salt thereof;
(iii) about 0.1% (w/w) to about 10% (w/w) benzyl alcohol;
(iv) sodium chloride; and
(v) a pH adjuster comprising sodium hydroxide and/or hydrochloric acid.

In various embodiments, a liquid pharmaceutical formulation comprises:
(i) about 0.025 mg/mL to about 65 mg/mL bumetanide, or a pharmaceutically acceptable salt thereof;
(ii) about 10 mM to about 250 mM tromethamine, or a pharmaceutically acceptable salt thereof; and
(iii) about 0.01% (w/w) to about 2% (w/w) sodium metabisulfite.

### Exemplary Torsemide Liquid Pharmaceutical Formulations

In various embodiments, provided herein are liquid pharmaceutical formulations for treating a disease or disorder described herein (e.g., congestion, edema, fluid overload, or hypertension) in a patient in need thereof, which comprise a therapeutically effective amount of torsemide, and a pharmaceutically acceptable excipient. The liquid pharmaceutical formulation can include, among other things, a pharmaceutically acceptable pH adjuster, an antioxidant, an osmolarity adjuster, and/or a pharmaceutically acceptable buffer as described herein.

In various embodiments, a liquid pharmaceutical formulation comprises:
(i) about 0.5 mg/mL to about 125 mg/mL torsemide, or a pharmaceutically acceptable salt thereof; and
(ii) about 10 mM to about 500 mM of a pharmaceutically acceptable buffer.

In various embodiments, a liquid pharmaceutical formulation comprises:
(i) torsemide, or a pharmaceutically acceptable salt thereof, in a therapeutically effective amount; and
(ii) tromethamine, or a pharmaceutically acceptable salt thereof.

In various embodiments, a liquid pharmaceutical formulation comprises:
(i) about 0.5 mg/mL to about 125 mg/mL torsemide, or a pharmaceutically acceptable salt thereof; and
(ii) about 10 mM to about 250 mM tromethamine, or a pharmaceutically acceptable salt thereof.

In various embodiments, a liquid pharmaceutical formulation comprises:
(i) torsemide, or a pharmaceutically acceptable salt thereof, in a therapeutically effective amount;
(ii) a pharmaceutically acceptable buffer; and
(iii) a pharmaceutically acceptable pH adjuster.

In various embodiments, a liquid pharmaceutical formulation comprises:
(i) torsemide, or a pharmaceutically acceptable salt thereof, in a therapeutically effective amount;
(ii) a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) a pharmaceutically acceptable pH adjuster selected from the group consisting of acetic acid, citric acid, fumaric acid, hydrochloric acid, malic acid, nitric acid, phosphoric acid, propionic acid, sulfuric acid, tartaric acid, ammonia solution, ammonium carbonate, diethanolamine, potassium hydroxide, sodium bicarbonate, sodium borate, sodium carbonate, sodium hydroxide, trolamine, and combinations thereof.

In various embodiments, a liquid pharmaceutical formulation comprises:
(i) about 0.5 mg/mL to about 125 mg/mL torsemide, or a pharmaceutically acceptable salt thereof;
(ii) about 10 mM to about 500 mM of a pharmaceutically acceptable buffer; and
(iii) a pharmaceutically acceptable pH adjuster.

In various embodiments, a liquid pharmaceutical formulation comprises:
(i) about 0.5 mg/mL to about 125 mg/mL torsemide, or a pharmaceutically acceptable salt thereof;
(ii) about 10 mM to about 500 mM of a pharmaceutically acceptable buffer; and
(iii) a pharmaceutically acceptable pH adjuster.

In various embodiments, a liquid pharmaceutical formulation comprises:
(i) about 0.5 mg/mL to about 125 mg/mL torsemide, or a pharmaceutically acceptable salt thereof;
(ii) about 10 mM to about 500 mM of a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) a pharmaceutically acceptable pH adjuster selected from the group consisting of acetic acid, citric acid, fumaric acid, hydrochloric acid, malic acid, nitric acid, phosphoric acid, propionic acid, sulfuric acid, tartaric acid, ammonia solution, ammonium carbonate, diethanolamine, potassium hydroxide, sodium bicarbonate, sodium borate, sodium carbonate, sodium hydroxide, trolamine, and combinations thereof.

In various embodiments, a liquid pharmaceutical formulation comprises:
(i) about 0.5 mg/mL to about 125 mg/mL torsemide, or a pharmaceutically acceptable salt thereof;
(ii) about 10 mM to about 250 mM tromethamine, or a pharmaceutically acceptable salt thereof; and
(iii) a pharmaceutically acceptable pH adjuster comprising sodium hydroxide and hydrochloric acid.

In various embodiments, a liquid pharmaceutical formulation comprises:
(i) torsemide, or a pharmaceutically acceptable salt thereof, in a therapeutically effective amount;
(ii) a pharmaceutically acceptable buffer; and
(iii) an antioxidant.

In various embodiments, a liquid pharmaceutical formulation comprises:
(i) torsemide, or a pharmaceutically acceptable salt thereof, in a therapeutically effective amount;
(ii) a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) an antioxidant selected from the group consisting of sodium metabisulfite, sodium bisulfite monothioglycerol, methionine, ethylenediaminetetraacetic acid (EDTA), and combinations thereof.

In various embodiments, a liquid pharmaceutical formulation comprises:
(i) about 0.5 mg/mL to about 125 mg/mL torsemide, or a pharmaceutically acceptable salt thereof;
(ii) about 10 mM to about 500 mM of a pharmaceutically acceptable buffer; and
(iii) about 0.01% (w/w) to about 2% (w/w) of an antioxidant.

In various embodiments, a liquid pharmaceutical formulation comprises:
(i) about 0.5 mg/mL to about 125 mg/mL torsemide, or a pharmaceutically acceptable salt thereof;
(ii) about 10 mM to about 500 mM of a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) about 0.01% (w/w) to about 2% (w/w) of an antioxidant selected from the group consisting of sodium metabisulfite, sodium bisulfite monothioglycerol, methionine, ethylenediaminetetraacetic acid (EDTA), and combinations thereof.

In various embodiments, a liquid pharmaceutical formulation comprises:
(i) about 0.5 mg/mL to about 125 mg/mL torsemide, or a pharmaceutically acceptable salt thereof;
(ii) about 10 mM to about 250 mM tromethamine, or a pharmaceutically acceptable salt thereof; and
(iii) about 0.01% (w/w) to about 2% (w/w) sodium metabisulfite.

In various embodiments, a liquid pharmaceutical formulation comprises:
(i) torsemide, or a pharmaceutically acceptable salt thereof, in a therapeutically effective amount;
(ii) a pharmaceutically acceptable buffer; and
(iii) an osmolarity adjuster.

In various embodiments, a liquid pharmaceutical formulation comprises:
(i) torsemide, or a pharmaceutically acceptable salt thereof, in a therapeutically effective amount;
(ii) a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) an osmolarity adjuster selected from the group consisting of sodium chloride, potassium chloride, isosorbide, mannitol, xylitol, and combinations thereof.

In various embodiments, a liquid pharmaceutical formulation comprises:
(i) about 0.5 mg/mL to about 125 mg/mL torsemide, or a pharmaceutically acceptable salt thereof;
(ii) about 10 mM to about 500 mM of a pharmaceutically acceptable buffer; and
(iii) an osmolarity adjuster.

In various embodiments, a liquid pharmaceutical formulation comprises:
(i) about 0.5 mg/mL to about 125 mg/mL torsemide, or a pharmaceutically acceptable salt thereof;
(ii) about 10 mM to about 500 mM of a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof, and
(iii) an osmolarity adjuster selected from the group consisting of sodium chloride, potassium chloride, isosorbide, mannitol, xylitol, and combinations thereof.

In various embodiments, a liquid pharmaceutical formulation comprises:
(i) about 0.5 mg/mL to about 125 mg/mL torsemide, or a pharmaceutically acceptable salt thereof;
(ii) about 10 mM to about 250 mM tromethamine, or a pharmaceutically acceptable salt thereof; and
(iii) sodium chloride.

In various embodiments, a liquid pharmaceutical formulation comprises:
(i) torsemide, or a pharmaceutically acceptable salt thereof, in a therapeutically effective amount;
(ii) a pharmaceutically acceptable buffer; and
(iii) one or more additional pharmaceutically acceptable excipients.

In various embodiments, a liquid pharmaceutical formulation comprises:
(i) torsemide, or a pharmaceutically acceptable salt thereof, in a therapeutically effective amount;
(ii) a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) one or more additional pharmaceutically acceptable excipients selected from the group consisting of ethanol, benzyl alcohol, glycerin, N-methyl-pyrrolidone (NMP), sodium chloride, sodium carbonate, mannitol, lactose, dextrose, a polyethylene glycol (PEG), propylene glycol, a polysorbate, a polyvinylpyrrolidone (PVP), a cyclodextrin or a derivative thereof, vitamin E or a derivative thereof, and combinations thereof.

In various embodiments, a liquid pharmaceutical formulation comprises:
(i) about 0.5 mg/mL to about 125 mg/mL torsemide, or a pharmaceutically acceptable salt thereof;
(ii) about 10 mM to about 500 mM of a pharmaceutically acceptable buffer; and
(iii) about 0.1% (w/w) to about 30% (w/w) one or more additional pharmaceutically acceptable excipients.

In various embodiments, a liquid pharmaceutical formulation comprises:
(i) about 0.5 mg/mL to about 125 mg/mL torsemide, or a pharmaceutically acceptable salt thereof;
(ii) about 10 mM to about 500 mM of a pharmaceutically acceptable buffer comprising a buffering agent selected from the group consisting of histidine, a citrate salt, sodium phosphate, potassium phosphate, tromethamine or a pharmaceutically acceptable salt thereof; and
(iii) about 0.1% (w/w) to about 30% (w/w) one or more additional pharmaceutically acceptable excipients selected from the group consisting of ethanol, benzyl alcohol, glycerin, N-methyl-pyrrolidone (NMP), sodium chloride, sodium carbonate, mannitol, lactose, dextrose, a polyethylene glycol (PEG), propylene glycol, a polysorbate, a polyvinylpyrrolidone (PVP), a cyclodextrin or a derivative thereof, vitamin E or a derivative thereof, and combinations thereof.

In various embodiments, a liquid pharmaceutical formulation comprises:
(i) about 0.5 mg/mL to about 125 mg/mL torsemide, or a pharmaceutically acceptable salt thereof;
(ii) about 10 mM to about 250 mM tromethamine, or a pharmaceutically acceptable salt thereof; and
(iii) about 0.1% (w/w) to about 30% (w/w) one or more additional pharmaceutically acceptable excipients.

In various embodiments, a liquid pharmaceutical formulation comprises:
(i) about 0.5 mg/mL to about 125 mg/mL torsemide, or a pharmaceutically acceptable salt thereof;
(ii) about 10 mM to about 250 mM tromethamine, or a pharmaceutically acceptable salt thereof; and
(iii) about 0.1% (w/w) to about 30% (w/w) one or more additional pharmaceutically acceptable excipients selected from the group consisting of ethanol, benzyl alcohol, glycerin, N-methyl-pyrrolidone (NMP), sodium chloride, sodium carbonate, mannitol, lactose, dextrose, a polyethylene glycol (PEG), propylene glycol, a polysorbate, a polyvinylpyrrolidone (PVP), a cyclodextrin or a derivative thereof, vitamin E or a derivative thereof, and combinations thereof.

In various embodiments, a liquid pharmaceutical formulation comprises:
(i) torsemide, or a pharmaceutically acceptable salt thereof, in a therapeutically effective amount; and
(ii) a cyclodextrin or a derivative thereof.

In various embodiments, a liquid pharmaceutical formulation comprises:
(i) about 0.5 mg/mL to about 125 mg/mL torsemide, or a pharmaceutically acceptable salt thereof; and
(ii) about 5% to about 50% (w/w) of a cyclodextrin or a derivative thereof.

In various embodiments, a liquid pharmaceutical formulation comprises:
(i) torsemide, or a pharmaceutically acceptable salt thereof, in a therapeutically effective amount;
(ii) a pharmaceutically acceptable buffer; and
(iii) a cyclodextrin or a derivative thereof.

In various embodiments, a liquid pharmaceutical formulation comprises:
(i) about 0.5 mg/mL to about 125 mg/mL torsemide, or a pharmaceutically acceptable salt thereof;
(ii) about 10 mM to about 500 mM of a pharmaceutically acceptable buffer; and
(iii) about 5% to about 50% (w/w) of a cyclodextrin or a derivative thereof.

In various embodiments, a liquid pharmaceutical formulation comprises:
(i) torsemide, or a pharmaceutically acceptable salt thereof, in a therapeutically effective amount;
(ii) tromethamine, or a pharmaceutically acceptable salt thereof; and
(iii) a cyclodextrin or a derivative thereof.

In various embodiments, a liquid pharmaceutical formulation comprises:
(i) about 0.5 mg/mL to about 125 mg/mL torsemide, or a pharmaceutically acceptable salt thereof;
(ii) about 10 mM to about 250 mM tromethamine, or a pharmaceutically acceptable salt thereof; and
(iii) about 5% to about 50% (w/w) of a cyclodextrin or a derivative thereof.

In various embodiments, a liquid pharmaceutical formulation comprises:
(i) torsemide, or a pharmaceutically acceptable salt thereof, in a therapeutically effective amount; and
(ii) benzyl alcohol.

In various embodiments, a liquid pharmaceutical formulation comprises:
(i) about 0.5 mg/mL to about 125 mg/mL torsemide, or a pharmaceutically acceptable salt thereof; and
(ii) about 0.1% (w/w) to about 10% (w/w) benzyl alcohol.

In various embodiments, a liquid pharmaceutical formulation comprises:
(i) torsemide, or a pharmaceutically acceptable salt thereof, in a therapeutically effective amount;
(ii) a pharmaceutically acceptable buffer; and
(iii) benzyl alcohol

In various embodiments, a liquid pharmaceutical formulation comprises:
(i) about 0.5 mg/mL to about 125 mg/mL torsemide, or a pharmaceutically acceptable salt thereof;
(ii) about 10 mM to about 500 mM of a pharmaceutically acceptable buffer; and
(iii) about 0.1% (w/w) to about 10% (w/w) benzyl alcohol.

In various embodiments, a liquid pharmaceutical formulation comprises:
(i) torsemide, or a pharmaceutically acceptable salt thereof, in a therapeutically effective amount;
(ii) tromethamine, or a pharmaceutically acceptable salt thereof; and
(iii) benzyl alcohol.

In various embodiments, a liquid pharmaceutical formulation comprises:
(i) about 0.5 mg/mL to about 125 mg/mL torsemide, or a pharmaceutically acceptable salt thereof;
(ii) about 10 mM to about 250 mM tromethamine, or a pharmaceutically acceptable salt thereof; and
(iii) about 0.1% (w/w) to about 10% (w/w) benzyl alcohol.

In various embodiments, a liquid pharmaceutical formulation comprises:
(i) torsemide, or a pharmaceutically acceptable salt thereof, in a therapeutically effective amount;
(ii) tromethamine, or a pharmaceutically acceptable salt thereof;
(iii) benzyl alcohol;
(iv) sodium chloride; and
(v) a pH adjuster comprising sodium hydroxide and/or hydrochloric acid.

In various embodiments, a liquid pharmaceutical formulation comprises:
(i) about 0.5 mg/mL to about 125 mg/mL torsemide, or a pharmaceutically acceptable salt thereof;
(ii) about 10 mM to about 250 mM tromethamine, or a pharmaceutically acceptable salt thereof;
(iii) about 0.1% (w/w) to about 10% (w/w) benzyl alcohol;
(iv) sodium chloride; and
(v) a pH adjuster comprising sodium hydroxide and/or hydrochloric acid.

In various embodiments, a liquid pharmaceutical formulation comprises:
(i) about 0.5 mg/mL to about 125 mg/mL torsemide, or a pharmaceutically acceptable salt thereof;
(ii) about 10 mM to about 250 mM tromethamine, or a pharmaceutically acceptable salt thereof; and
(iii) about 0.01% (w/w) to about 2% (w/w) sodium metabisulfite.

### Unit Liquid Pharmaceutical Formulations of Furosemide

As described herein, in one aspect, the invention provides unit liquid pharmaceutical formulations of a loop diuretic (e.g., bumetanide, furosemide, or torsemide).

In various embodiments, a liquid pharmaceutical formulation generally comprises:
(i) a loop diuretic, or a pharmaceutically acceptable salt thereof, in a therapeutically effective amount; and
(ii) a pharmaceutically acceptable excipient, for example, a pharmaceutically acceptable pH adjuster, an antioxidant, an osmolarity adjuster, and/or a pharmaceutically acceptable buffer as described herein.

### Loop Diuretics

In certain embodiments, the amount of the loop diuretic in a unit liquid pharmaceutical formulation described herein can be from about 0.025 mg to about 250 mg, from about 0.1 mg to about 250 mg, from about 1 mg to about 250 mg, from about 5 mg to about 250 mg, from about 10 mg to about 250 mg, from about 20 mg to about 250 mg, from about 30 mg to about 250 mg, from about 40 mg to about 250 mg, from about 50 mg to about 250 mg, from about 60 mg to about 250 mg, from about 70 mg to about 250 mg, from about 80 mg to about 250 mg, from about 90 mg to about 250 mg, from about 100 mg to about 250 mg, from about 120 mg to about 250 mg, from about 140 mg to about 250 mg, from about 160 mg to about 250 mg, from about 180 mg to about 250 mg, from about 200 mg to about 250 mg, from about 225 mg to about 250 mg, from about 0.025 mg to about 225 mg, from about 0.025 mg to about 200 mg, from about 0.025 mg to about 180 mg, from about 0.025 mg to about 160 mg, from about 0.025 mg to about 140 mg, from about 0.025 mg to about 120 mg, from about 0.025 mg to about 100 mg, from about 0.025 mg to about 90 mg, from about 0.025 mg to about 80 mg, from about 0.025 mg to about 70 mg, from about 0.025 mg to about 60 mg, from about 0.025 mg to about 50 mg, from about 0.025 mg to about 40 mg, from about 0.025 mg to about 30 mg, from about 0.025 mg to about 20 mg, from about 0.025 mg to about 10 mg, from about 0.025 mg to about 5 mg, from about 0.025 mg to about 1 mg, from about 0.025 mg to about 0.1 mg, from about 0.1 mg to about 225 mg, from about 0.1 mg to about 200 mg, from about 0.1 mg to about 180 mg, from about 0.1 mg to about 160 mg, from about 0.1 mg to about 140 mg, from about 0.1 mg to about 120 mg, from about 0.1 mg to about 100 mg, from about 0.1 mg to about 90 mg, from about 0.1 mg to about 80 mg, from about 0.1 mg to about 70 mg, from about 0.1 mg to about 60 mg, from about 0.1 mg to about 50 mg, from about 0.1 mg to about 40 mg, from about 0.1 mg to about 30 mg, from about 0.1 mg to about 20 mg, from about 0.1 mg to about 10 mg, from about 0.1 mg to about 5 mg, from about 0.1 mg to about 1 mg, from about 1 mg to about 225 mg, from about 1 mg to about 200 mg, from about 1 mg to about 180 mg, from about 1 mg to about 160 mg, from about 1 mg to about 140 mg, from about 1 mg to about 120 mg, from about 1 mg to about 100 mg, from about 1 mg to about 90 mg, from about 1 mg to about 80 mg, from about 1 mg to about 70 mg, from about 1 mg to about 60 mg, from about 1 mg to about 50 mg, from about 1 mg to about 40 mg, from about 1 mg to about 30 mg, from about 1 mg to about 20 mg, from about 1 mg to about 10 mg, from about 1 mg to about 5 mg, from about 5 mg to about 225 mg, from about 5 mg to about 200 mg, from about 5 mg to about 180 mg, from about 5 mg to about 160 mg, from about 5 mg to about 140 mg, from about 5 mg to about 120 mg, from about 5 mg to about 100 mg, from about 5 mg to about 90 mg, from about 5 mg to about 80 mg, from about 5 mg to about 70 mg, from about 5 mg to about 60 mg, from about 5 mg to about 50 mg, from about 5 mg to about 40 mg, from about 5 mg to about 30 mg, from about 5 mg to about 20 mg, from about 5 mg to about 10 mg, from about 10 mg to about 225 mg, from about 10 mg to about 200 mg, from about 10 mg to about 180 mg, from about 10 mg to about 160 mg, from about 10 mg to about 140 mg, from about 10 mg to about 120 mg, from about 10 mg to about 100 mg, from about 10 mg to about 90 mg, from about 10 mg to about 80 mg, from about 10 mg to about 70 mg, from about 10 mg to about 60 mg, from about 10 mg to about 50 mg, from about 10 mg to about 40 mg, from about 10 mg to about 30 mg, from about 10 mg to about 20 mg, from about 20 mg to about 225 mg, from about 20 mg to about 200 mg, from about 20 mg to about 180 mg, from about 20 mg to about 160 mg, from about 20 mg to about 140 mg, from about 20 mg to about 120 mg, from about 20 mg to about 100 mg, from about 20 mg to about 90 mg, from about 20 mg to about 80 mg, from about 20 mg to about 70 mg, from about 20 mg to about 60 mg, from about 20 mg to about 50 mg, from about 20 mg to about 40 mg, from about 20 mg to about 30 mg, from about 30 mg to about 225 mg, from about 30 mg to about 200 mg, from about 30 mg to about 180 mg, from about 30 mg to about 160 mg, from about 30 mg to about 140 mg, from about 30 mg to about 120 mg, from about 30 mg to about 100 mg, from about 30 mg to about 90 mg, from about 30 mg to about 80 mg, from about 30 mg to about 70 mg, from about 30 mg to about 60 mg, from about 30 mg to about 50 mg, from about 30 mg to about 40 mg, from about 40 mg to about 225 mg, from about 40 mg to about 200 mg, from about 40 mg to about 180 mg, from about 40 mg to about 160 mg, from about 40 mg to about 140 mg, from about 40 mg to about 120 mg, from about 40 mg to about 100 mg, from about 40 mg to about 90 mg, from about 40 mg to about 80 mg, from about 40 mg to about 70 mg, from about 40 mg to about 60 mg, from about 40 mg to about 50 mg, from about 50 mg to about 225 mg, from about 50 mg to about 200 mg, from about 50 mg to about 180 mg, from about 50 mg to about 160 mg, from about 50 mg to about 140 mg, from about 50 mg to about 120 mg, from about 50 mg to about 100 mg, from about 50 mg to about 90 mg, from about 50 mg to about 80 mg, from about 50 mg to about 70 mg, from about 50 mg to about 60 mg, from about 60 mg to about 225 mg, from about 60 mg to about 200 mg, from about 60 mg to about 180 mg, from about 60 mg to about 160 mg, from about 60 mg to about 140 mg, from about 60 mg to about 120 mg, from about 60 mg to about 100 mg, from about 60 mg to about 90 mg, from about 60 mg to about 80 mg, from about 60 mg to about 70 mg, from about 70 mg to about 225 mg, from about 70 mg to about 200 mg, from about 70 mg to about 180 mg, from about 70 mg to about 160 mg, from about 70 mg to about 140 mg, from about 70 mg to about 120 mg, from about 70 mg to about 100 mg, from about 70 mg to about 90 mg, from about 70 mg to about 80 mg, from about 70 mg to about 225 mg, from about 80 mg to about 200 mg, from about 80 mg to about 180 mg, from about 80 mg to about 160 mg, from about 80 mg to about 140 mg, from about 80 mg to about 120 mg, from about 80 mg to about 100 mg, from about 80 mg to about 90 mg, from about 70 mg to about 225 mg, from about 90 mg to about 200 mg, from about 90 mg to about 180 mg, from about 90 mg to about 160 mg, from about 90 mg to about 140 mg, from about 90 mg to about 120 mg, from about 90 mg to about 100 mg, from about 100 mg to about 225 mg, from about 100 mg to about 200 mg, from about 100 mg to about 180 mg, from about 100 mg to about 160 mg, from about 100 mg to about 140 mg, from about 100 mg to about 120 mg, from about 120 mg to about 225 mg, from about 120 mg to about 200 mg, from about 120 mg to about 180 mg, from about 120 mg to about 160 mg, from about 120 mg to about 140 mg, from about 140 mg to about 225 mg, from about 140 mg to about 200 mg, from about 140 mg to about 180 mg, from about 140 mg to about 160 mg, from about 160 mg to about 225 mg, from about 160 mg to about 200 mg, from about 160 mg to about 180 mg, from about 180 mg to about 225 mg, from about 180 mg to about 200 mg, or from about 200 mg to about 225 mg. In certain embodiments, the amount of the loop diuretic in a unit liquid pharmaceutical formulation described herein can be from about 0.025 mg to about 250 mg.

In certain embodiments, the amount of the loop diuretic in the unit liquid pharmaceutical formulations described herein can be greater than about 0.025 mg, greater than about 0.1 mg, greater than about 1 mg, greater than about 5 mg, greater than about 10 mg, greater than about 20 mg, greater than about 30 mg, greater than about 40 mg, greater than about 50 mg, greater than about 60 mg, greater than about 70 mg, greater than about 80 mg, greater than about 90 mg, greater than about 100 mg, greater than about 110 mg, greater than about 120 mg, greater than about 130 mg, greater than about 140 mg, greater than about 150 mg, greater than about 160 mg, greater than about 170 mg, greater than about 180 mg, greater than about 190 mg, greater than about 200 mg, greater than about 210 mg, greater than about 220 mg, greater than about 230 mg, greater than about 240 mg, or greater than about 250 mg.

In certain embodiments, the amount of the loop diuretic in the unit liquid pharmaceutical formulations described herein can be about 0.025 mg, about 0.05 mg, about 0.075 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 22 mg, about 24 mg, about 26 mg, about 28 mg, about 30 mg, about 32 mg, about 34 mg, about 36 mg, about 38 mg, about 40 mg, about 42 mg, about 44 mg, about 46 mg, about 48 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, or about 250 mg.

In certain embodiments, the loop diuretic is selected from the group consisting of furosemide, bumetanide, ethacrynic acid, torsemide, or a pharmaceutically acceptable salt thereof. In certain embodiments, the loop diuretic is furosemide, or a pharmaceutically acceptable salt thereof. In certain embodiments, the loop diuretic is bumetanide, or a pharmaceutically acceptable salt thereof. In certain embodiments, the loop diuretic is ethacrynic acid, or a pharmaceutically acceptable salt thereof. In certain embodiments, the loop diuretic is torsemide, or a pharmaceutically acceptable salt thereof.

In certain embodiments, the amount of furosemide in a unit liquid pharmaceutical formulation described herein can be from about 1 mg to about 250 mg, from about 5 mg to about 250 mg, from about 10 mg to about 250 mg, from about 20 mg to about 250 mg, from about 30 mg to about 250 mg, from about 40 mg to about 250 mg, from about 50 mg to about 250 mg, from about 60 mg to about 250 mg, from about 70 mg to about 250 mg, from about 80 mg to about 250 mg, from about 90 mg to about 250 mg, from about 100 mg to about 250 mg, from about 120 mg to about 250 mg, from about 140 mg to about 250 mg, from about 160 mg to about 250 mg, from about 180 mg to about 250 mg, from about 200 mg to about 250 mg, from about 225 mg to about 250 mg, from about 1 mg to about 225 mg, from about 1 mg to about 200 mg, from about 1 mg to about 180 mg, from about 1 mg to about 160 mg, from about 1 mg to about 140 mg, from about 1 mg to about 120 mg, from about 1 mg to about 100 mg, from about 1 mg to about 90 mg, from about 1 mg to about 80 mg, from about 1 mg to about 70 mg, from about 1 mg to about 60 mg, from about 1 mg to about 50 mg, from about 1 mg to about 40 mg, from about 1 mg to about 30 mg, from about 1 mg to about 20 mg, from about 1 mg to about 10 mg, from about 1 mg to about 5 mg, from about 5 mg to about 225 mg, from about 5 mg to about 200 mg, from about 5 mg to about 180 mg, from about 5 mg to about 160 mg, from about 5 mg to about 140 mg, from about 5 mg to about 120 mg, from about 5 mg to about 100 mg, from about 5 mg to about 90 mg, from about 5 mg to about 80 mg, from about 5 mg to about 70 mg, from about 5 mg to about 60 mg, from about 5 mg to about 50 mg, from about 5 mg to about 40 mg, from about 5 mg to about 30 mg, from about 5 mg to about 20 mg, from about 5 mg to about mg, from about 10 mg to about 225 mg, from about 10 mg to about 200 mg, from about 10 mg to about 180 mg, from about 10 mg to about 160 mg, from about 10 mg to about 140 mg, from about 10 mg to about 120 mg, from about 10 mg to about 100 mg, from about 10 mg to 10 about 90 mg, from about 10 mg to about 80 mg, from about 10 mg to about 70 mg, from about 10 mg to about 60 mg, from about 10 mg to about 50 mg, from about 10 mg to about 40 mg, from about 10 mg to about 30 mg, from about 10 mg to about 20 mg, from about 20 mg to about 225 mg, from about 20 mg to about 200 mg, from about 20 mg to about 180 mg, from about 20 mg to about 160 mg, from about 20 mg to about 140 mg, from about 20 mg to about 120 mg, from about 20 mg to about 100 mg, from about 20 mg to about 90 mg, from about 20 mg to about 80 mg, from about 20 mg to about 70 mg, from about 20 mg to about 60 mg, from about mg to about 50 mg, from about 20 mg to about 40 mg, from about 20 mg to about 30 mg, from about 30 mg to about 225 mg, from about 30 mg to about 200 mg, from about 30 mg to about 180 mg, from about 30 mg to about 160 mg, from about 30 mg to about 140 mg, from about 30 mg to about 120 mg, from about 30 mg to about 100 mg, from about 30 mg to about 90 mg, from about 30 mg to about 80 mg, from about 30 mg to about 70 mg, from about 30 mg to about 60 mg, from about 30 mg to about 50 mg, from about 30 mg to about 40 mg, from about 40 mg to about 225 mg, from about 40 mg to about 200 mg, from about 40 mg to about 180 mg, from about 40 mg to about 160 mg, from about 40 mg to about 140 mg, from about 40 mg to about 120 mg, from about 40 mg to about 100 mg, from about 40 mg to about 90 mg, from about 40 mg to about 80 mg, from about 40 mg to about 70 mg, from about 40 mg to about 60 mg, from about 40 mg to about 50 mg, from about 50 mg to about 225 mg, from about 50 mg to about 200 mg, from about 50 mg to about 180 mg, from about 50 mg to about 160 mg, from about 50 mg to about 140 mg, from about 50 mg to about 120 mg, from about 50 mg to about 100 mg, from about 50 mg to about 90 mg, from about 50 mg to about 80 mg, from about 50 mg to about 70 mg, from about 50 mg to about 60 mg, from about 60 mg to about 225 mg, from about 60 mg to about 200 mg, from about 60 mg to about 180 mg, from about 60 mg to about 160 mg, from about 60 mg to about 140 mg, from about 60 mg to about 120 mg, from about 60 mg to about 100 mg, from about 60 mg to about 90 mg, from about 60 mg to about 80 mg, from about 60 mg to about 70 mg, from about 70 mg to about 225 mg, from about 70 mg to about 200 mg, from about 70 mg to about 180 mg, from about 70 mg to about 160 mg, from about 70 mg to about 140 mg, from about 70 mg to about 120 mg, from about 70 mg to about 100 mg, from about 70 mg to about 90 mg, from about 70 mg to about 80 mg, from about 70 mg to about 225 mg, from about 80 mg to about 200 mg, from about 80 mg to about 180 mg, from about 80 mg to about 160 mg, from about 80 mg to about 140 mg, from about 80 mg to about 120 mg, from about 80 mg to about 100 mg, from about 80 mg to about 90 mg, from about 70 mg to about 225 mg, from about 90 mg to about 200 mg, from about 90 mg to about 180 mg, from about 90 mg to about 160 mg, from about 90 mg to about 140 mg, from about 90 mg to about 120 mg, from about 90 mg to about 100 mg, from about 100 mg to about 225 mg, from about 100 mg to about 200 mg, from about 100 mg to about 180 mg, from about 100 mg to about 160 mg, from about 100 mg to about 140 mg, from about 100 mg to about 120 mg, from about 120 mg to about 225 mg, from about 120 mg to about 200 mg, from about 120 mg to about 180 mg, from about 120 mg to about 160 mg, from about 120 mg to about 140 mg, from about 140 mg to about 225 mg, from about 140 mg to about 200 mg, from about 140 mg to about 180 mg, from about 140 mg to about 160 mg, from about 160 mg to about 225 mg, from about 160 mg to about 200 mg, from about 160 mg to about 180 mg, from about 180 mg to about 225 mg, from about 180 mg to about 200 mg, or from about 200 mg to about 225 mg. In certain embodiments, the amount of furosemide in a unit liquid pharmaceutical formulation described herein can be from about 1 mg to about 250 mg.

In certain embodiments, the amount of furosemide in the unit liquid pharmaceutical formulations described herein can be greater than about 1 mg, greater than about 5 mg, greater than about 10 mg, greater than about 20 mg, greater than about 30 mg, greater than about 40 mg, greater than about 50 mg, greater than about 60 mg, greater than about 70 mg, greater than about 80 mg, greater than about 90 mg, greater than about 100 mg, greater than about 110 mg, greater than about 120 mg, greater than about 130 mg, greater than about 140 mg, greater than about 150 mg, greater than about 160 mg, greater than about 170 mg, greater than about 180 mg, greater than about 190 mg, greater than about 200 mg, greater than about 210 mg, greater than about 220 mg, greater than about 230 mg, greater than about 240 mg, or greater than about 250 mg.

In certain embodiments, the amount of furosemide in the unit liquid pharmaceutical formulations described herein can be about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 22 mg, about 24 mg, about 26 mg, about 28 mg, about 30 mg, about 32 mg, about 34 mg, about 36 mg, about 38 mg, about 40 mg, about 42 mg, about 44 mg, about 46 mg, about 48 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, or about 250 mg. In certain embodiments, the amount of furosemide in the unit liquid pharmaceutical formulations described herein can be about 80 mg.

In certain embodiments, the amount of bumetanide in a unit liquid pharmaceutical formulation described herein can be from about 0.025 mg to about 65 mg, from about 0.1 mg to about 65 mg, from about 0.5 mg to about 65 mg, from about 1 mg to about 65 mg, from about 5 mg to about 65 mg, from about 10 mg to about 65 mg, from about 20 mg to about 65 mg, from about 30 mg to about 65 mg, from about 40 mg to about 65 mg, from about 50 mg to about 65 mg, from about 60 mg to about 65 mg, from about 0.025 mg to about 60 mg, from about 0.025 mg to about 50 mg, from about 0.025 mg to about 40 mg, from about 0.025 mg to about 30 mg, from about 0.025 mg to about 20 mg, from about 0.025 mg to about 10 mg, from about 0.025 mg to about 5 mg, from about 0.025 mg to about 1 mg, from about 0.025 mg to about 0.5 mg, from about 0.025 mg to about 0.1 mg, from about 0.1 mg to about 60 mg, from about 0.1 mg to about 50 mg, from about 0.1 mg to about 40 mg, from about 0.1 mg to about 30 mg, from about 0.1 mg to about 20 mg, from about 0.1 mg to about 10 mg, from about 0.1 mg to about 5 mg, from about 0.1 mg to about 1 mg, from about 0.5 mg to about 60 mg, from about 0.5 mg to about 50 mg, from about 0.5 mg to about 40 mg, from about 0.5 mg to about 30 mg, from about 0.5 mg to about 20 mg, from about 0.5 mg to about 10 mg, from about 0.5 mg to about 10 mg, from about 0.5 mg to about 5 mg, from about 0.5 mg to about 1 mg, from about 1 mg to about 60 mg, from about 1 mg to about 50 mg, from about 1 mg to about 40 mg, from about 1 mg to about 30 mg, from about 1 mg to about 20 mg, from about 1 mg to about 10 mg, from about 1 mg to about 5 mg, from about 5 mg to about 60 mg, from about 5 mg to about 50 mg, from about 5 mg to about 40 mg, from about 5 mg to about 30 mg, from about 5 mg to about 20 mg, from about 5 mg to about 10 mg, from about 10 mg to about 60 mg, from about 10 mg to about 50 mg, from about 10 mg to about 40 mg, from about 10 mg to about 30 mg, from about 10 mg to about 20 mg, from about 20 mg to about 60 mg, from about 20 mg to about 50 mg, from about 20 mg to about 40 mg, from about 20 mg to about 30 mg, from about 30 mg to about 60 mg, from about 30 mg to about 50 mg, from about 30 mg to about 40 mg, from about 40 mg to about 60 mg, from about 40 mg to about 50 mg, or from about 50 mg to about 60 mg. In certain embodiments, the amount of bumetanide in a unit liquid pharmaceutical formulation described herein can be from about 0.025 mg to about 65 mg.

In certain embodiments, the amount of bumetanide in the unit liquid pharmaceutical formulations described herein can be greater than about 0.025 mg, greater than about 1 mg, greater than about 5 mg, greater than about 10 mg, greater than about 20 mg, greater than about 30 mg, greater than about 40 mg, greater than about 50 mg, or greater than about 60 mg.

In certain embodiments, the amount of bumetanide in the unit liquid pharmaceutical formulations described herein can be about 0.025 mg, about 0.05 mg, about 0.075 mg, about 0.1 mg, about 0.25 mg, about 0.5 mg, about 0.75 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 22 mg, about 24 mg, about 26 mg, about 28 mg, about 30 mg, about 32 mg, about 34 mg, about 36 mg, about 38 mg, about 40 mg, about 42 mg, about 44 mg, about 46 mg, about 48 mg, about 50 mg, about 55 mg, about 60 mg, or about 65 mg.

In certain embodiments, the amount of torsemide in a unit liquid pharmaceutical formulation described herein can be from about 0.5 mg to about 125 mg, from about 1 mg to about 125 mg, from about 5 mg to about 125 mg, from about 10 mg to about 125 mg, from about 20 mg to about 125 mg, from about 30 mg to about 125 mg, from about 40 mg to about 125 mg, from about 50 mg to about 125 mg, from about 75 mg to about 125 mg, from about 100 mg to about 125 mg, from about 0.5 mg to about 100 mg, from about 0.5 mg to about 75 mg, from about 0.5 mg to about 50 mg, from about 0.5 mg to about 40 mg, from about 0.5 mg to about 30 mg, from about 0.5 mg to about 20 mg, from about 0.5 mg to about 10 mg, from about 0.5 mg to about 5 mg, from about 0.5 mg to about 1 mg, from about 1 mg to about 100 mg, from about 1 mg to about 75 mg, from about 1 mg to about 50 mg, from about 1 mg to about 40 mg, from about 1 mg to about 30 mg, from about 1 mg to about 20 mg, from about 1 mg to about 10 mg, from about 1 mg to about 5 mg, from about 5 mg to about 100 mg, from about 5 mg to about 75 mg, from about 5 mg to about 50 mg, from about 5 mg to about 40 mg, from about 5 mg to about 30 mg, from about 5 mg to about 20 mg, from about 5 mg to about 10 mg, from about 10 mg to about 100 mg, from about 10 mg to about 75 mg, from about 10 mg to about 50 mg, from about 10 mg to about 40 mg, from about 10 mg to about 30 mg, from about 10 mg to about 20 mg, from about 20 mg to about 100 mg, from about 20 mg to about 75 mg, from about 20 mg to about 50 mg, from about 20 mg to about 40 mg, from about 20 mg to about 30 mg, from about 30 mg to about 100 mg, from about 30 mg to about 75 mg, from about 30 mg to about 50 mg, from about 30 mg to about 40 mg, from about 40 mg to about 100 mg, from about 40 mg to about 75 mg, from about 40 mg to about 50 mg, from about 50 mg to about 100 mg, from about 50 mg to about 75 mg, or from about 75 mg to about 100 mg.

In certain embodiments, the amount of torsemide in the liquid pharmaceutical formulations described herein can be greater than about 0.5 mg, greater than about 1 mg, greater than about 5 mg, greater than about 10 mg, greater than about 20 mg, greater than about 30 mg, greater than about 40 mg, greater than about 50 mg, greater than about 60 mg, greater than about 70 mg, greater than about 80 mg, greater than about 90 mg, greater than about 100 mg, or greater than about 125 mg.

In certain embodiments, the amount of torsemide in the unit liquid pharmaceutical formulations described herein can be about 0.5 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 60 mg, about 70 mg, about 80 mg, about 90 mg, about 100 mg, or about 125 mg.

### Excipients and More for Unit Liquid Pharmaceutical Formulations

It should be understood that the excipients, amounts and concentrations, and other conditions and parameters described herein for liquid pharmaceutical formulations apply equally to unit liquid pharmaceutical formulations described herein as if those excipients, amounts and concentrations, and other conditions and parameters were written here in this section but for brevity, will not be repeated.

More specifically, the pH, the pharmaceutically acceptable buffers and concentrations thereof, the pharmaceutically acceptable pH adjusters and concentrations thereof, the antioxidants and concentrations thereof, the osmolarity, and additional pharmaceutically acceptable excipients (such as benzyl alcohol, cyclodextrins and cyclodextrin derivatives) and concentrations thereof, described herein for a liquid pharmaceutical formulation apply equally to a unit liquid pharmaceutical formulation described herein.

### Exemplary Furosemide Unit Liquid Pharmaceutical Formulations

In various embodiments, provided herein are unit liquid pharmaceutical formulations for treating a disease or disorder described herein (e.g., congestion, edema, fluid overload, or hypertension) in a patient in need thereof, which comprise a therapeutically effective amount of furosemide, and a pharmaceutically acceptable excipient. The unit liquid pharmaceutical formulation can include, among other things, a pharmaceutically acceptable pH adjuster, an antioxidant, an osmolarity adjuster, and/or a pharmaceutically acceptable buffer as described herein.

In various embodiments, a unit liquid pharmaceutical formulation comprises:
(i) about 1 mg to about 250 mg furosemide, or a pharmaceutically acceptable salt thereof; and
(ii) about 10 mM to about 500 mM of a pharmaceutically acceptable buffer.

In various embodiments, a unit liquid pharmaceutical formulation comprises:
(i) furosemide, or a pharmaceutically acceptable salt thereof, in a therapeutically effective amount; and
(ii) tromethamine, or a pharmaceutically acceptable salt thereof.

In various embodiments, a unit liquid pharmaceutical formulation comprises:
(i) about 1 mg to about 250 mg furosemide, or a pharmaceutically acceptable salt thereof; and
(ii) about 10 mM to about 250 mM tromethamine, or a pharmaceutically acceptable salt thereof.

In various embodiments, a unit liquid pharmaceutical formulation comprises:
(i) furosemide, or a pharmaceutically acceptable salt thereof, in a therapeutically effective amount;
(ii) a pharmaceutically acceptable buffer; and
(iii) a pharmaceutically acceptable pH adjuster.

In various embodiments, a unit liquid pharmaceutical formulation comprises:
(i) furosemide, or a pharmaceutically acceptable salt thereof, in a therapeutically effective amount;
(ii) a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) a pharmaceutically acceptable pH adjuster selected from the group consisting of acetic acid, citric acid, fumaric acid, hydrochloric acid, malic acid, nitric acid, phosphoric acid, propionic acid, sulfuric acid, tartaric acid, ammonia solution, ammonium carbonate, diethanolamine, potassium hydroxide, sodium bicarbonate, sodium borate, sodium carbonate, sodium hydroxide, trolamine, and combinations thereof.

In various embodiments, a unit liquid pharmaceutical formulation comprises:
(i) about 1 mg to about 250 mg furosemide, or a pharmaceutically acceptable salt thereof;
(ii) about 10 mM to about 500 mM of a pharmaceutically acceptable buffer; and
(iii) a pharmaceutically acceptable pH adjuster.

In various embodiments, a unit liquid pharmaceutical formulation comprises:
(i) about 1 mg to about 250 mg furosemide, or a pharmaceutically acceptable salt thereof;
(ii) about 10 mM to about 500 mM of a pharmaceutically acceptable buffer; and
(iii) a pharmaceutically acceptable pH adjuster.

In various embodiments, a unit liquid pharmaceutical formulation comprises:
(i) about 1 mg to about 250 mg furosemide, or a pharmaceutically acceptable salt thereof;
(ii) about 10 mM to about 500 mM of a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) a pharmaceutically acceptable pH adjuster selected from the group consisting of acetic acid, citric acid, fumaric acid, hydrochloric acid, malic acid, nitric acid, phosphoric acid, propionic acid, sulfuric acid, tartaric acid, ammonia solution, ammonium carbonate, diethanolamine, potassium hydroxide, sodium bicarbonate, sodium borate, sodium carbonate, sodium hydroxide, trolamine, and combinations thereof.

In various embodiments, a unit liquid pharmaceutical formulation comprises:
(i) about 1 mg to about 250 mg furosemide, or a pharmaceutically acceptable salt thereof;
(ii) about 10 mM to about 250 mM tromethamine, or a pharmaceutically acceptable salt thereof; and
(iii) a pharmaceutically acceptable pH adjuster comprising sodium hydroxide and hydrochloric acid.

In various embodiments, a unit liquid pharmaceutical formulation comprises:
(i) furosemide, or a pharmaceutically acceptable salt thereof, in a therapeutically effective amount;
(ii) a pharmaceutically acceptable buffer; and
(iii) an antioxidant.

In various embodiments, a unit liquid pharmaceutical formulation comprises:
(i) furosemide, or a pharmaceutically acceptable salt thereof, in a therapeutically effective amount;
(ii) a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) an antioxidant selected from the group consisting of sodium metabisulfite, sodium bisulfite monothioglycerol, methionine, ethylenediaminetetraacetic acid (EDTA), and combinations thereof.

In various embodiments, a unit liquid pharmaceutical formulation comprises:
(i) about 1 mg to about 250 mg furosemide, or a pharmaceutically acceptable salt thereof;
(ii) about 10 mM to about 500 mM of a pharmaceutically acceptable buffer; and
(iii) about 0.01% (w/w) to about 2% (w/w) of an antioxidant.

In various embodiments, a unit liquid pharmaceutical formulation comprises:
(i) about 1 mg to about 250 mg furosemide, or a pharmaceutically acceptable salt thereof;
(ii) about 10 mM to about 500 mM of a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) about 0.01% (w/w) to about 2% (w/w) of an antioxidant selected from the group consisting of sodium metabisulfite, sodium bisulfite monothioglycerol, methionine, ethylenediaminetetraacetic acid (EDTA), and combinations thereof.

In various embodiments, a unit liquid pharmaceutical formulation comprises:
(i) about 1 mg to about 250 mg furosemide, or a pharmaceutically acceptable salt thereof;
(ii) about 10 mM to about 250 mM tromethamine, or a pharmaceutically acceptable salt thereof; and
(iii) about 0.01% (w/w) to about 2% (w/w) sodium metabisulfite.

In various embodiments, a unit liquid pharmaceutical formulation comprises:
(i) furosemide, or a pharmaceutically acceptable salt thereof, in a therapeutically effective amount;
(ii) a pharmaceutically acceptable buffer; and
(iii) an osmolarity adjuster.

In various embodiments, a unit liquid pharmaceutical formulation comprises:
(i) furosemide, or a pharmaceutically acceptable salt thereof, in a therapeutically effective amount;
(ii) a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) an osmolarity adjuster selected from the group consisting of sodium chloride, potassium chloride, isosorbide, mannitol, xylitol, and combinations thereof.

In various embodiments, a unit liquid pharmaceutical formulation comprises:
(i) about 1 mg to about 250 mg furosemide, or a pharmaceutically acceptable salt thereof;
(ii) about 10 mM to about 500 mM of a pharmaceutically acceptable buffer; and
(iii) an osmolarity adjuster.

In various embodiments, a unit liquid pharmaceutical formulation comprises:
(i) about 1 mg to about 250 mg furosemide, or a pharmaceutically acceptable salt thereof;
(ii) about 10 mM to about 500 mM of a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof, and
(iii) an osmolarity adjuster selected from the group consisting of sodium chloride, potassium chloride, isosorbide, mannitol, xylitol, and combinations thereof.

In various embodiments, a unit liquid pharmaceutical formulation comprises:
(i) about 1 mg to about 250 mg furosemide, or a pharmaceutically acceptable salt thereof;
(ii) about 10 mM to about 250 mM tromethamine, or a pharmaceutically acceptable salt thereof; and
(iii) sodium chloride.

In various embodiments, a unit liquid pharmaceutical formulation comprises:
(i) furosemide, or a pharmaceutically acceptable salt thereof, in a therapeutically effective amount;
(ii) a pharmaceutically acceptable buffer; and
(iii) one or more additional pharmaceutically acceptable excipients.

In various embodiments, a unit liquid pharmaceutical formulation comprises:
(i) furosemide, or a pharmaceutically acceptable salt thereof, in a therapeutically effective amount;
(ii) a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) one or more additional pharmaceutically acceptable excipients selected from the group consisting of ethanol, benzyl alcohol, glycerin, N-methyl-pyrrolidone (NMP), sodium chloride, sodium carbonate, mannitol, lactose, dextrose, a polyethylene glycol (PEG), propylene glycol, a polysorbate, a polyvinylpyrrolidone (PVP), a cyclodextrin or a derivative thereof, vitamin E or a derivative thereof, and combinations thereof.

In various embodiments, a unit liquid pharmaceutical formulation comprises:
(i) about 1 mg to about 250 mg furosemide, or a pharmaceutically acceptable salt thereof;
(ii) about 10 mM to about 500 mM of a pharmaceutically acceptable buffer; and
(iii) about 0.1% (w/w) to about 30% (w/w) one or more additional pharmaceutically acceptable excipients.

In various embodiments, a unit liquid pharmaceutical formulation comprises:
(i) about 1 mg to about 250 mg furosemide, or a pharmaceutically acceptable salt thereof;
(ii) about 10 mM to about 500 mM of a pharmaceutically acceptable buffer comprising a buffering agent selected from the group consisting of histidine, a citrate salt, sodium phosphate, potassium phosphate, tromethamine or a pharmaceutically acceptable salt thereof; and
(iii) about 0.1% (w/w) to about 30% (w/w) one or more additional pharmaceutically acceptable excipients selected from the group consisting of ethanol, benzyl alcohol, glycerin, N-methyl-pyrrolidone (NMP), sodium chloride, sodium carbonate, mannitol, lactose, dextrose, a polyethylene glycol (PEG), propylene glycol, a polysorbate, a polyvinylpyrrolidone (PVP), a cyclodextrin or a derivative thereof, vitamin E or a derivative thereof, and combinations thereof.

In various embodiments, a unit liquid pharmaceutical formulation comprises:
(i) about 1 mg to about 250 mg furosemide, or a pharmaceutically acceptable salt thereof;
(ii) about 10 mM to about 250 mM tromethamine, or a pharmaceutically acceptable salt thereof; and
(iii) about 0.1% (w/w) to about 30% (w/w) one or more additional pharmaceutically acceptable excipients.

In various embodiments, a unit liquid pharmaceutical formulation comprises:
(i) about 1 mg to about 250 mg furosemide, or a pharmaceutically acceptable salt thereof;
(ii) about 10 mM to about 250 mM tromethamine, or a pharmaceutically acceptable salt thereof; and
(iii) about 0.1% (w/w) to about 30% (w/w) one or more additional pharmaceutically acceptable excipients selected from the group consisting of ethanol, benzyl alcohol, glycerin, N-methyl-pyrrolidone (NMP), sodium chloride, sodium carbonate, mannitol, lactose, dextrose, a polyethylene glycol (PEG), propylene glycol, a polysorbate, a polyvinylpyrrolidone (PVP), a cyclodextrin or a derivative thereof, vitamin E or a derivative thereof, and combinations thereof.

In various embodiments, a unit liquid pharmaceutical formulation comprises:
(i) furosemide, or a pharmaceutically acceptable salt thereof, in a therapeutically effective amount; and
(ii) a cyclodextrin or a derivative thereof.

In various embodiments, a unit liquid pharmaceutical formulation comprises:
(i) about 1 mg to about 250 mg furosemide, or a pharmaceutically acceptable salt thereof; and
(ii) about 5% to about 50% (w/w) of a cyclodextrin or a derivative thereof.

In various embodiments, a unit liquid pharmaceutical formulation comprises:
(i) furosemide, or a pharmaceutically acceptable salt thereof, in a therapeutically effective amount;
(ii) a pharmaceutically acceptable buffer; and
(iii) a cyclodextrin or a derivative thereof.

In various embodiments, a unit liquid pharmaceutical formulation comprises:
(i) about 1 mg to about 250 mg furosemide, or a pharmaceutically acceptable salt thereof;
(ii) about 10 mM to about 500 mM of a pharmaceutically acceptable buffer; and
(iii) about 5% to about 50% (w/w) of a cyclodextrin or a derivative thereof.

In various embodiments, a unit liquid pharmaceutical formulation comprises:
(i) furosemide, or a pharmaceutically acceptable salt thereof, in a therapeutically effective amount;
(ii) tromethamine, or a pharmaceutically acceptable salt thereof; and
(iii) a cyclodextrin or a derivative thereof.

In various embodiments, a unit liquid pharmaceutical formulation comprises:
(i) about 1 mg to about 250 mg furosemide, or a pharmaceutically acceptable salt thereof;
(ii) about 10 mM to about 250 mM tromethamine, or a pharmaceutically acceptable salt thereof; and
(iii) about 5% to about 50% (w/w) of a cyclodextrin or a derivative thereof.

In various embodiments, a unit liquid pharmaceutical formulation comprises:
(i) furosemide, or a pharmaceutically acceptable salt thereof, in a therapeutically effective amount; and
(ii) benzyl alcohol.

In various embodiments, a unit liquid pharmaceutical formulation comprises:
(i) about 1 mg to about 250 mg furosemide, or a pharmaceutically acceptable salt thereof; and
(ii) about 0.1% (w/w) to about 10% (w/w) benzyl alcohol.

In various embodiments, a unit liquid pharmaceutical formulation comprises:
(i) furosemide, or a pharmaceutically acceptable salt thereof, in a therapeutically effective amount;
(ii) a pharmaceutically acceptable buffer; and
(iii) benzyl alcohol.

In various embodiments, a unit liquid pharmaceutical formulation comprises:
(i) about 1 mg to about 250 mg furosemide, or a pharmaceutically acceptable salt thereof;
(ii) about 10 mM to about 500 mM of a pharmaceutically acceptable buffer; and
(iii) about 0.1% (w/w) to about 10% (w/w) benzyl alcohol.

In various embodiments, a unit liquid pharmaceutical formulation comprises:
(i) furosemide, or a pharmaceutically acceptable salt thereof, in a therapeutically effective amount;
(ii) tromethamine, or a pharmaceutically acceptable salt thereof; and
(iii) benzyl alcohol.

In various embodiments, a unit liquid pharmaceutical formulation comprises:
(i) about 1 mg to about 250 mg furosemide, or a pharmaceutically acceptable salt thereof;
(ii) about 10 mM to about 250 mM tromethamine, or a pharmaceutically acceptable salt thereof; and
(iii) about 0.1% (w/w) to about 10% (w/w) benzyl alcohol.

In various embodiments, a unit liquid pharmaceutical formulation comprises:
(i) furosemide, or a pharmaceutically acceptable salt thereof, in a therapeutically effective amount;
(ii) tromethamine, or a pharmaceutically acceptable salt thereof;
(iii) benzyl alcohol;
(iv) sodium chloride; and
(v) a pH adjuster comprising sodium hydroxide and/or hydrochloric acid.

In various embodiments, a unit liquid pharmaceutical formulation comprises:
(i) about 1 mg to about 250 mg furosemide, or a pharmaceutically acceptable salt thereof;
(ii) about 10 mM to about 250 mM tromethamine, or a pharmaceutically acceptable salt thereof;
(iii) about 0.1% (w/w) to about 10% (w/w) benzyl alcohol;
(iv) sodium chloride; and
(v) a pH adjuster comprising sodium hydroxide and/or hydrochloric acid.

In various embodiments, a unit liquid pharmaceutical formulation comprises:
(i) about 1 mg to about 250 mg furosemide, or a pharmaceutically acceptable salt thereof;
(ii) about 10 mM to about 250 mM tromethamine, or a pharmaceutically acceptable salt thereof; and
(iii) about 0.01% (w/w) to about 2% (w/w) sodium metabisulfite.

### Exemplary Bumetanide Unit Liquid Pharmaceutical Formulations

In various embodiments, provided herein are unit liquid pharmaceutical formulations for treating a disease or disorder described herein (e.g., congestion, edema, fluid overload, or hypertension) in a patient in need thereof, which comprise a therapeutically effective amount of bumetanide, and a pharmaceutically acceptable excipient. The unit liquid pharmaceutical formulation can include, among other things, a pharmaceutically acceptable pH adjuster, an antioxidant, an osmolarity adjuster, and/or a pharmaceutically acceptable buffer as described herein.

In various embodiments, a unit liquid pharmaceutical formulation comprises:
(i) about 0.025 mg to about 65 mg bumetanide, or a pharmaceutically acceptable salt thereof; and
(ii) about 10 mM to about 500 mM of a pharmaceutically acceptable buffer.

In various embodiments, a unit liquid pharmaceutical formulation comprises:
(i) bumetanide, or a pharmaceutically acceptable salt thereof, in a therapeutically effective amount; and
(ii) tromethamine, or a pharmaceutically acceptable salt thereof.

In various embodiments, a unit liquid pharmaceutical formulation comprises:
(i) about 0.025 mg to about 65 mg bumetanide, or a pharmaceutically acceptable salt thereof; and
(ii) about 10 mM to about 250 mM tromethamine, or a pharmaceutically acceptable salt thereof.

In various embodiments, a unit liquid pharmaceutical formulation comprises:
(i) bumetanide, or a pharmaceutically acceptable salt thereof, in a therapeutically effective amount;
(ii) a pharmaceutically acceptable buffer; and
(iii) a pharmaceutically acceptable pH adjuster.

In various embodiments, a unit liquid pharmaceutical formulation comprises:
(i) bumetanide, or a pharmaceutically acceptable salt thereof, in a therapeutically effective amount;
(ii) a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) a pharmaceutically acceptable pH adjuster selected from the group consisting of acetic acid, citric acid, fumaric acid, hydrochloric acid, malic acid, nitric acid, phosphoric acid, propionic acid, sulfuric acid, tartaric acid, ammonia solution, ammonium carbonate, diethanolamine, potassium hydroxide, sodium bicarbonate, sodium borate, sodium carbonate, sodium hydroxide, trolamine, and combinations thereof.

In various embodiments, a unit liquid pharmaceutical formulation comprises:
(i) about 0.025 mg to about 65 mg bumetanide, or a pharmaceutically acceptable salt thereof;
(ii) about 10 mM to about 500 mM of a pharmaceutically acceptable buffer; and
(iii) a pharmaceutically acceptable pH adjuster.

In various embodiments, a unit liquid pharmaceutical formulation comprises:
(i) about 0.025 mg to about 65 mg bumetanide, or a pharmaceutically acceptable salt thereof;
(ii) about 10 mM to about 500 mM of a pharmaceutically acceptable buffer; and
(iii) a pharmaceutically acceptable pH adjuster.

In various embodiments, a unit liquid pharmaceutical formulation comprises:
(i) about 0.025 mg to about 65 mg bumetanide, or a pharmaceutically acceptable salt thereof;
(ii) about 10 mM to about 500 mM of a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof, and
(iii) a pharmaceutically acceptable pH adjuster selected from the group consisting of acetic acid, citric acid, fumaric acid, hydrochloric acid, malic acid, nitric acid, phosphoric acid, propionic acid, sulfuric acid, tartaric acid, ammonia solution, ammonium carbonate, diethanolamine, potassium hydroxide, sodium bicarbonate, sodium borate, sodium carbonate, sodium hydroxide, trolamine, and combinations thereof.

In various embodiments, a unit liquid pharmaceutical formulation comprises:
(i) about 0.025 mg to about 65 mg bumetanide, or a pharmaceutically acceptable salt thereof;
(ii) about 10 mM to about 250 mM tromethamine, or a pharmaceutically acceptable salt thereof; and
(iii) a pharmaceutically acceptable pH adjuster comprising sodium hydroxide and hydrochloric acid.

In various embodiments, a unit liquid pharmaceutical formulation comprises:
(i) bumetanide, or a pharmaceutically acceptable salt thereof, in a therapeutically effective amount;
(ii) a pharmaceutically acceptable buffer; and
(iii) an antioxidant.

In various embodiments, a unit liquid pharmaceutical formulation comprises:
(i) bumetanide, or a pharmaceutically acceptable salt thereof, in a therapeutically effective amount;
(ii) a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) an antioxidant selected from the group consisting of sodium metabisulfite, sodium bisulfite monothioglycerol, methionine, ethylenediaminetetraacetic acid (EDTA), and combinations thereof.

In various embodiments, a unit liquid pharmaceutical formulation comprises:
(i) about 0.025 mg to about 65 mg bumetanide, or a pharmaceutically acceptable salt thereof;
(ii) about 10 mM to about 500 mM of a pharmaceutically acceptable buffer; and
(iii) about 0.01% (w/w) to about 2% (w/w) of an antioxidant.

In various embodiments, a unit liquid pharmaceutical formulation comprises:
(i) about 0.025 mg to about 65 mg bumetanide, or a pharmaceutically acceptable salt thereof;
(ii) about 10 mM to about 500 mM of a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) about 0.01% (w/w) to about 2% (w/w) of an antioxidant selected from the group consisting of sodium metabisulfite, sodium bisulfite monothioglycerol, methionine, ethylenediaminetetraacetic acid (EDTA), and combinations thereof.

In various embodiments, a unit liquid pharmaceutical formulation comprises:
(i) about 0.025 mg to about 65 mg bumetanide, or a pharmaceutically acceptable salt thereof;
(ii) about 10 mM to about 250 mM tromethamine, or a pharmaceutically acceptable salt thereof; and
(iii) about 0.01% (w/w) to about 2% (w/w) sodium metabisulfite.

In various embodiments, a unit liquid pharmaceutical formulation comprises:
(i) bumetanide, or a pharmaceutically acceptable salt thereof, in a therapeutically effective amount;
(ii) a pharmaceutically acceptable buffer; and
(iii) an osmolarity adjuster.

In various embodiments, a unit liquid pharmaceutical formulation comprises:
(i) bumetanide, or a pharmaceutically acceptable salt thereof, in a therapeutically effective amount;
(ii) a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) an osmolarity adjuster selected from the group consisting of sodium chloride, potassium chloride, isosorbide, mannitol, xylitol, and combinations thereof.

In various embodiments, a unit liquid pharmaceutical formulation comprises:
(i) about 0.025 mg to about 65 mg bumetanide, or a pharmaceutically acceptable salt thereof;
(ii) about 10 mM to about 500 mM of a pharmaceutically acceptable buffer; and
(iii) an osmolarity adjuster.

In various embodiments, a unit liquid pharmaceutical formulation comprises:
(i) about 0.025 mg to about 65 mg bumetanide, or a pharmaceutically acceptable salt thereof;
(ii) about 10 mM to about 500 mM of a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) an osmolarity adjuster selected from the group consisting of sodium chloride, potassium chloride, isosorbide, mannitol, xylitol, and combinations thereof.

In various embodiments, a unit liquid pharmaceutical formulation comprises:
(i) about 0.025 mg to about 65 mg bumetanide, or a pharmaceutically acceptable salt thereof;
(ii) about 10 mM to about 250 mM tromethamine, or a pharmaceutically acceptable salt thereof; and
(iii) sodium chloride.

In various embodiments, a unit liquid pharmaceutical formulation comprises:
(i) bumetanide, or a pharmaceutically acceptable salt thereof, in a therapeutically effective amount;
(ii) a pharmaceutically acceptable buffer; and
(iii) one or more additional pharmaceutically acceptable excipients.

In various embodiments, a unit liquid pharmaceutical formulation comprises:
(i) bumetanide, or a pharmaceutically acceptable salt thereof, in a therapeutically effective amount;
(ii) a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) one or more additional pharmaceutically acceptable excipients selected from the group consisting of ethanol, benzyl alcohol, glycerin, N-methyl-pyrrolidone (NMP), sodium chloride, sodium carbonate, mannitol, lactose, dextrose, a polyethylene glycol (PEG), propylene glycol, a polysorbate, a polyvinylpyrrolidone (PVP), a cyclodextrin or a derivative thereof, vitamin E or a derivative thereof, and combinations thereof.

In various embodiments, a unit liquid pharmaceutical formulation comprises:
(i) about 0.025 mg to about 65 mg bumetanide, or a pharmaceutically acceptable salt thereof;
(ii) about 10 mM to about 500 mM of a pharmaceutically acceptable buffer; and
(iii) about 0.1% (w/w) to about 30% (w/w) one or more additional pharmaceutically acceptable excipients.

In various embodiments, a unit liquid pharmaceutical formulation comprises:
(i) about 0.025 mg to about 65 mg bumetanide, or a pharmaceutically acceptable salt thereof;
(ii) about 10 mM to about 500 mM of a pharmaceutically acceptable buffer comprising a buffering agent selected from the group consisting of histidine, a citrate salt, sodium phosphate, potassium phosphate, tromethamine or a pharmaceutically acceptable salt thereof; and
(iii) about 0.1% (w/w) to about 30% (w/w) one or more additional pharmaceutically acceptable excipients selected from the group consisting of ethanol, benzyl alcohol, glycerin, N-methyl-pyrrolidone (NMP), sodium chloride, sodium carbonate, mannitol, lactose, dextrose, a polyethylene glycol (PEG), propylene glycol, a polysorbate, a polyvinylpyrrolidone (PVP), a cyclodextrin or a derivative thereof, vitamin E or a derivative thereof, and combinations thereof.

In various embodiments, a unit liquid pharmaceutical formulation comprises:
(i) about 0.025 mg to about 65 mg bumetanide, or a pharmaceutically acceptable salt thereof;
(ii) about 10 mM to about 250 mM tromethamine, or a pharmaceutically acceptable salt thereof; and
(iii) about 0.1% (w/w) to about 30% (w/w) one or more additional pharmaceutically acceptable excipients.

In various embodiments, a unit liquid pharmaceutical formulation comprises:
(i) about 0.025 mg to about 65 mg bumetanide, or a pharmaceutically acceptable salt thereof;
(ii) about 10 mM to about 250 mM tromethamine, or a pharmaceutically acceptable salt thereof; and
(iii) about 0.1% (w/w) to about 30% (w/w) one or more additional pharmaceutically acceptable excipients selected from the group consisting of ethanol, benzyl alcohol, glycerin, N-methyl-pyrrolidone (NMP), sodium chloride, sodium carbonate, mannitol, lactose, dextrose, a polyethylene glycol (PEG), propylene glycol, a polysorbate, a polyvinylpyrrolidone (PVP), a cyclodextrin or a derivative thereof, vitamin E or a derivative thereof, and combinations thereof.

In various embodiments, a unit liquid pharmaceutical formulation comprises:
(i) bumetanide, or a pharmaceutically acceptable salt thereof, in a therapeutically effective amount; and
(ii) a cyclodextrin or a derivative thereof.

In various embodiments, a unit liquid pharmaceutical formulation comprises:
(i) about 0.025 mg to about 65 mg bumetanide, or a pharmaceutically acceptable salt thereof; and
(ii) about 5% to about 50% (w/w) of a cyclodextrin or a derivative thereof.

In various embodiments, a unit liquid pharmaceutical formulation comprises:
(i) bumetanide, or a pharmaceutically acceptable salt thereof, in a therapeutically effective amount;
(ii) a pharmaceutically acceptable buffer; and
(iii) a cyclodextrin or a derivative thereof.

In various embodiments, a unit liquid pharmaceutical formulation comprises:
(i) about 0.025 mg to about 65 mg bumetanide, or a pharmaceutically acceptable salt thereof;
(ii) about 10 mM to about 500 mM of a pharmaceutically acceptable buffer; and
(iii) about 5% to about 50% (w/w) of a cyclodextrin or a derivative thereof.

In various embodiments, a unit liquid pharmaceutical formulation comprises:
(i) bumetanide, or a pharmaceutically acceptable salt thereof, in a therapeutically effective amount;
(ii) tromethamine, or a pharmaceutically acceptable salt thereof; and
(iii) a cyclodextrin or a derivative thereof.

In various embodiments, a unit liquid pharmaceutical formulation comprises:
(i) about 0.025 mg to about 65 mg bumetanide, or a pharmaceutically acceptable salt thereof;
(ii) about 10 mM to about 250 mM tromethamine, or a pharmaceutically acceptable salt thereof; and
(iii) about 5% to about 50% (w/w) of a cyclodextrin or a derivative thereof.

In various embodiments, a unit liquid pharmaceutical formulation comprises:
(i) bumetanide, or a pharmaceutically acceptable salt thereof, in a therapeutically effective amount; and
(ii) benzyl alcohol.

In various embodiments, a unit liquid pharmaceutical formulation comprises:
(i) about 0.025 mg to about 65 mg bumetanide, or a pharmaceutically acceptable salt thereof; and
(ii) about 0.1% (w/w) to about 10% (w/w) benzyl alcohol.

In various embodiments, a unit liquid pharmaceutical formulation comprises:
(i) bumetanide, or a pharmaceutically acceptable salt thereof, in a therapeutically effective amount;
(ii) a pharmaceutically acceptable buffer; and
(iii) benzyl alcohol.

In various embodiments, a unit liquid pharmaceutical formulation comprises:
(i) about 0.025 mg to about 65 mg bumetanide, or a pharmaceutically acceptable salt thereof;
(ii) about 10 mM to about 500 mM of a pharmaceutically acceptable buffer; and
(iii) about 0.1% (w/w) to about 10% (w/w) benzyl alcohol.

In various embodiments, a unit liquid pharmaceutical formulation comprises:
(i) bumetanide, or a pharmaceutically acceptable salt thereof, in a therapeutically effective amount;
(ii) tromethamine, or a pharmaceutically acceptable salt thereof; and
(iii) benzyl alcohol.

In various embodiments, a unit liquid pharmaceutical formulation comprises:
(i) about 0.025 mg to about 65 mg bumetanide, or a pharmaceutically acceptable salt thereof;
(ii) about 10 mM to about 250 mM tromethamine, or a pharmaceutically acceptable salt thereof; and
(iii) about 0.1% (w/w) to about 10% (w/w) benzyl alcohol.

In various embodiments, a unit liquid pharmaceutical formulation comprises:
(i) bumetanide, or a pharmaceutically acceptable salt thereof, in a therapeutically effective amount;
(ii) tromethamine, or a pharmaceutically acceptable salt thereof;
(iii) benzyl alcohol;
(iv) sodium chloride; and
(v) a pH adjuster comprising sodium hydroxide and/or hydrochloric acid.

In various embodiments, a unit liquid pharmaceutical formulation comprises:
(i) about 0.025 mg to about 65 mg bumetanide, or a pharmaceutically acceptable salt thereof;
(ii) about 10 mM to about 250 mM tromethamine, or a pharmaceutically acceptable salt thereof;
(iii) about 0.1% (w/w) to about 10% (w/w) benzyl alcohol;
(iv) sodium chloride; and
(v) a pH adjuster comprising sodium hydroxide and/or hydrochloric acid.

In various embodiments, a unit liquid pharmaceutical formulation comprises:
(i) about 0.025 mg to about 65 mg bumetanide, or a pharmaceutically acceptable salt thereof;
(ii) about 10 mM to about 250 mM tromethamine, or a pharmaceutically acceptable salt thereof; and
(iii) about 0.01% (w/w) to about 2% (w/w) sodium metabisulfite.

### Exemplary Torsemide Unit Liquid Pharmaceutical Formulations

In various embodiments, provided herein are unit liquid pharmaceutical formulations for treating a disease or disorder described herein (e.g., congestion, edema, fluid overload, or hypertension) in a patient in need thereof, which comprise a therapeutically effective amount of torsemide, and a pharmaceutically acceptable excipient. The unit liquid pharmaceutical formulation can include, among other things, a pharmaceutically acceptable pH adjuster, an antioxidant, an osmolarity adjuster, and/or a pharmaceutically acceptable buffer as described herein.

In various embodiments, a unit liquid pharmaceutical formulation comprises:
(i) about 0.5 mg to about 125 mg torsemide, or a pharmaceutically acceptable salt thereof; and
(ii) about 10 mM to about 500 mM of a pharmaceutically acceptable buffer.

In various embodiments, a unit liquid pharmaceutical formulation comprises:
(i) torsemide, or a pharmaceutically acceptable salt thereof, in a therapeutically effective amount; and
(ii) tromethamine, or a pharmaceutically acceptable salt thereof.

In various embodiments, a unit liquid pharmaceutical formulation comprises:
(i) about 0.5 mg to about 125 mg torsemide, or a pharmaceutically acceptable salt thereof; and
(ii) about 10 mM to about 250 mM tromethamine, or a pharmaceutically acceptable salt thereof.

In various embodiments, a unit liquid pharmaceutical formulation comprises:
(i) torsemide, or a pharmaceutically acceptable salt thereof, in a therapeutically effective amount;
(ii) a pharmaceutically acceptable buffer; and
(iii) a pharmaceutically acceptable pH adjuster.

In various embodiments, a unit liquid pharmaceutical formulation comprises:
(i) torsemide, or a pharmaceutically acceptable salt thereof, in a therapeutically effective amount;
(ii) a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) a pharmaceutically acceptable pH adjuster selected from the group consisting of acetic acid, citric acid, fumaric acid, hydrochloric acid, malic acid, nitric acid, phosphoric acid, propionic acid, sulfuric acid, tartaric acid, ammonia solution, ammonium carbonate, diethanolamine, potassium hydroxide, sodium bicarbonate, sodium borate, sodium carbonate, sodium hydroxide, trolamine, and combinations thereof.

In various embodiments, a unit liquid pharmaceutical formulation comprises:
(i) about 0.5 mg to about 125 mg torsemide, or a pharmaceutically acceptable salt thereof;
(ii) about 10 mM to about 500 mM of a pharmaceutically acceptable buffer; and
(iii) a pharmaceutically acceptable pH adjuster.

In various embodiments, a unit liquid pharmaceutical formulation comprises:
(i) about 0.5 mg to about 125 mg torsemide, or a pharmaceutically acceptable salt thereof;
(ii) about 10 mM to about 500 mM of a pharmaceutically acceptable buffer; and
(iii) a pharmaceutically acceptable pH adjuster.

In various embodiments, a unit liquid pharmaceutical formulation comprises:
(i) about 0.5 mg to about 125 mg torsemide, or a pharmaceutically acceptable salt thereof;
(ii) about 10 mM to about 500 mM of a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof, and
(iii) a pharmaceutically acceptable pH adjuster selected from the group consisting of acetic acid, citric acid, fumaric acid, hydrochloric acid, malic acid, nitric acid, phosphoric acid, propionic acid, sulfuric acid, tartaric acid, ammonia solution, ammonium carbonate, diethanolamine, potassium hydroxide, sodium bicarbonate, sodium borate, sodium carbonate, sodium hydroxide, trolamine, and combinations thereof.

In various embodiments, a unit liquid pharmaceutical formulation comprises:
(i) about 0.5 mg to about 125 mg torsemide, or a pharmaceutically acceptable salt thereof;
(ii) about 10 mM to about 250 mM tromethamine, or a pharmaceutically acceptable salt thereof; and
(iii) a pharmaceutically acceptable pH adjuster comprising sodium hydroxide and hydrochloric acid.

In various embodiments, a unit liquid pharmaceutical formulation comprises:
(i) torsemide, or a pharmaceutically acceptable salt thereof, in a therapeutically effective amount;
(ii) a pharmaceutically acceptable buffer; and
(iii) an antioxidant.

In various embodiments, a unit liquid pharmaceutical formulation comprises:
(i) torsemide, or a pharmaceutically acceptable salt thereof, in a therapeutically effective amount;
(ii) a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) an antioxidant selected from the group consisting of sodium metabisulfite, sodium bisulfite monothioglycerol, methionine, ethylenediaminetetraacetic acid (EDTA), and combinations thereof.

In various embodiments, a unit liquid pharmaceutical formulation comprises:
(i) about 0.5 mg to about 125 mg torsemide, or a pharmaceutically acceptable salt thereof;
(ii) about 10 mM to about 500 mM of a pharmaceutically acceptable buffer; and
(iii) about 0.01% (w/w) to about 2% (w/w) of an antioxidant.

In various embodiments, a unit liquid pharmaceutical formulation comprises:
(i) about 0.5 mg to about 125 mg torsemide, or a pharmaceutically acceptable salt thereof;
(ii) about 10 mM to about 500 mM of a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) about 0.01% (w/w) to about 2% (w/w) of an antioxidant selected from the group consisting of sodium metabisulfite, sodium bisulfite monothioglycerol, methionine, ethylenediaminetetraacetic acid (EDTA), and combinations thereof.

In various embodiments, a unit liquid pharmaceutical formulation comprises:
(i) about 0.5 mg to about 125 mg torsemide, or a pharmaceutically acceptable salt thereof;
(ii) about 10 mM to about 250 mM tromethamine, or a pharmaceutically acceptable salt thereof; and
(iii) about 0.01% (w/w) to about 2% (w/w) sodium metabisulfite.

In various embodiments, a unit liquid pharmaceutical formulation comprises:
(i) torsemide, or a pharmaceutically acceptable salt thereof, in a therapeutically effective amount;
(ii) a pharmaceutically acceptable buffer; and
(iii) an osmolarity adjuster.

In various embodiments, a unit liquid pharmaceutical formulation comprises:
(i) torsemide, or a pharmaceutically acceptable salt thereof, in a therapeutically effective amount;
(ii) a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) an osmolarity adjuster selected from the group consisting of sodium chloride, potassium chloride, isosorbide, mannitol, xylitol, and combinations thereof.

In various embodiments, a unit liquid pharmaceutical formulation comprises:
(i) about 0.5 mg to about 125 mg torsemide, or a pharmaceutically acceptable salt thereof;
(ii) about 10 mM to about 500 mM of a pharmaceutically acceptable buffer; and
(iii) an osmolarity adjuster.

In various embodiments, a unit liquid pharmaceutical formulation comprises:
(i) about 0.5 mg to about 125 mg torsemide, or a pharmaceutically acceptable salt thereof;
(ii) about 10 mM to about 500 mM of a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) an osmolarity adjuster selected from the group consisting of sodium chloride, potassium chloride, isosorbide, mannitol, xylitol, and combinations thereof.

In various embodiments, a unit liquid pharmaceutical formulation comprises:
(i) about 0.5 mg to about 125 mg torsemide, or a pharmaceutically acceptable salt thereof;
(ii) about 10 mM to about 250 mM tromethamine, or a pharmaceutically acceptable salt thereof; and
(iii) sodium chloride.

In various embodiments, a unit liquid pharmaceutical formulation comprises:
(i) torsemide, or a pharmaceutically acceptable salt thereof, in a therapeutically effective amount;
(ii) a pharmaceutically acceptable buffer; and
(iii) one or more additional pharmaceutically acceptable excipients.

In various embodiments, a unit liquid pharmaceutical formulation comprises:
(i) torsemide, or a pharmaceutically acceptable salt thereof, in a therapeutically effective amount;
(ii) a pharmaceutically acceptable buffer comprising a buffering agent selected from the group consisting of histidine, a citrate salt, sodium phosphate, potassium phosphate, tromethamine or a pharmaceutically acceptable salt thereof, and combinations thereof; and
(iii) one or more additional pharmaceutically acceptable excipients selected from the group consisting of ethanol, benzyl alcohol, glycerin, N-methyl-pyrrolidone (NMP), sodium chloride, sodium carbonate, mannitol, lactose, dextrose, a polyethylene glycol (PEG), propylene glycol, a polysorbate, a polyvinylpyrrolidone (PVP), a cyclodextrin or a derivative thereof, vitamin E or a derivative thereof, and combinations thereof.

In various embodiments, a unit liquid pharmaceutical formulation comprises:
(i) about 0.5 mg to about 125 mg torsemide, or a pharmaceutically acceptable salt thereof;
(ii) about 10 mM to about 500 mM of a pharmaceutically acceptable buffer; and
(iii) about 0.1% (w/w) to about 30% (w/w) one or more additional pharmaceutically acceptable excipients.

In various embodiments, a unit liquid pharmaceutical formulation comprises:

(i) about 0.5 mg to about 125 mg torsemide, or a pharmaceutically acceptable salt thereof;
(ii) about 10 mM to about 500 mM of a pharmaceutically acceptable buffer comprising a buffering agent selected from the group consisting of histidine, a citrate salt, sodium phosphate, potassium phosphate, tromethamine or a pharmaceutically acceptable salt thereof; and
(iii) about 0.1% (w/w) to about 30% (w/w) one or more additional pharmaceutically acceptable excipients selected from the group consisting of ethanol, benzyl alcohol, glycerin, N-methyl-pyrrolidone (NMP), sodium chloride, sodium carbonate, mannitol, lactose, dextrose, a polyethylene glycol (PEG), propylene glycol, a polysorbate, a polyvinylpyrrolidone (PVP), a cyclodextrin or a derivative thereof, vitamin E or a derivative thereof, and combinations thereof.

In various embodiments, a unit liquid pharmaceutical formulation comprises:
(i) about 0.5 mg to about 125 mg torsemide, or a pharmaceutically acceptable salt thereof;
(ii) about 10 mM to about 250 mM tromethamine, or a pharmaceutically acceptable salt thereof; and
(iii) about 0.1% (w/w) to about 30% (w/w) one or more additional pharmaceutically acceptable excipients.

In various embodiments, a unit liquid pharmaceutical formulation comprises:
(i) about 0.5 mg to about 125 mg torsemide, or a pharmaceutically acceptable salt thereof;
(ii) about 10 mM to about 250 mM tromethamine, or a pharmaceutically acceptable salt thereof; and
(iii) about 0.1% (w/w) to about 30% (w/w) one or more additional pharmaceutically acceptable excipients selected from the group consisting of ethanol, benzyl alcohol, glycerin, N-methyl-pyrrolidone (NMP), sodium chloride, sodium carbonate, mannitol, lactose, dextrose, a polyethylene glycol (PEG), propylene glycol, a polysorbate, a polyvinylpyrrolidone (PVP), a cyclodextrin or a derivative thereof, vitamin E or a derivative thereof, and combinations thereof.

In various embodiments, a unit liquid pharmaceutical formulation comprises:
(i) torsemide, or a pharmaceutically acceptable salt thereof, in a therapeutically effective amount; and
(ii) a cyclodextrin or a derivative thereof.

In various embodiments, a unit liquid pharmaceutical formulation comprises:
(i) about 0.5 mg to about 125 mg torsemide, or a pharmaceutically acceptable salt thereof; and
(ii) about 5% to about 50% (w/w) of a cyclodextrin or a derivative thereof.

In various embodiments, a unit liquid pharmaceutical formulation comprises:
(i) torsemide, or a pharmaceutically acceptable salt thereof, in a therapeutically effective amount;
(ii) a pharmaceutically acceptable buffer; and
(iii) a cyclodextrin or a derivative thereof.

In various embodiments, a unit liquid pharmaceutical formulation comprises:
(i) about 0.5 mg to about 125 mg torsemide, or a pharmaceutically acceptable salt thereof;
(ii) about 10 mM to about 500 mM of a pharmaceutically acceptable buffer; and
(iii) about 5% to about 50% (w/w) of a cyclodextrin or a derivative thereof.

In various embodiments, a unit liquid pharmaceutical formulation comprises:
(i) torsemide, or a pharmaceutically acceptable salt thereof, in a therapeutically effective amount;
(ii) tromethamine, or a pharmaceutically acceptable salt thereof; and
(iii) a cyclodextrin or a derivative thereof.

In various embodiments, a unit liquid pharmaceutical formulation comprises:
(i) about 0.5 mg to about 125 mg torsemide, or a pharmaceutically acceptable salt thereof;
(ii) about 10 mM to about 250 mM tromethamine, or a pharmaceutically acceptable salt thereof; and
(iii) about 5% to about 50% (w/w) of a cyclodextrin or a derivative thereof.

In various embodiments, a unit liquid pharmaceutical formulation comprises:
(i) torsemide, or a pharmaceutically acceptable salt thereof, in a therapeutically effective amount; and
(ii) benzyl alcohol.

In various embodiments, a unit liquid pharmaceutical formulation comprises:
(i) about 0.5 mg to about 125 mg torsemide, or a pharmaceutically acceptable salt thereof; and
(ii) about 0.1% (w/w) to about 10% (w/w) benzyl alcohol.

In various embodiments, a unit liquid pharmaceutical formulation comprises:
(i) torsemide, or a pharmaceutically acceptable salt thereof, in a therapeutically effective amount;
(ii) a pharmaceutically acceptable buffer; and
(iii) benzyl alcohol.

In various embodiments, a unit liquid pharmaceutical formulation comprises:
(i) about 0.5 mg to about 125 mg torsemide, or a pharmaceutically acceptable salt thereof;
(ii) about 10 mM to about 500 mM of a pharmaceutically acceptable buffer; and (iii) about 0.1% (w/w) to about 10% (w/w) benzyl alcohol.

In various embodiments, a unit liquid pharmaceutical formulation comprises:
(i) torsemide, or a pharmaceutically acceptable salt thereof, in a therapeutically effective amount;
(ii) tromethamine, or a pharmaceutically acceptable salt thereof; and
(iii) benzyl alcohol.

In various embodiments, a unit liquid pharmaceutical formulation comprises:
(i) about 0.5 mg to about 125 mg torsemide, or a pharmaceutically acceptable salt thereof;
(ii) about 10 mM to about 250 mM tromethamine, or a pharmaceutically acceptable salt thereof; and
(iii) about 0.1% (w/w) to about 10% (w/w) benzyl alcohol.

In various embodiments, a unit liquid pharmaceutical formulation comprises:
(i) torsemide, or a pharmaceutically acceptable salt thereof, in a therapeutically effective amount;
(ii) tromethamine, or a pharmaceutically acceptable salt thereof;
(iii) benzyl alcohol;
(iv) sodium chloride; and
(v) a pH adjuster comprising sodium hydroxide and/or hydrochloric acid.

In various embodiments, a unit liquid pharmaceutical formulation comprises:
(i) about 0.5 mg to about 125 mg torsemide, or a pharmaceutically acceptable salt thereof;
(ii) about 10 mM to about 250 mM tromethamine, or a pharmaceutically acceptable salt thereof;
(iii) about 0.1% (w/w) to about 10% (w/w) benzyl alcohol;
(iv) sodium chloride; and
(v) a pH adjuster comprising sodium hydroxide and/or hydrochloric acid.

In various embodiments, a unit liquid pharmaceutical formulation comprises:
(i) about 0.5 mg to about 125 mg torsemide, or a pharmaceutically acceptable salt thereof;
(ii) about 10 mM to about 250 mM tromethamine, or a pharmaceutically acceptable salt thereof; and
(iii) about 0.01% (w/w) to about 2% (w/w) sodium metabisulfite.

### Methods of Treatment

In one aspect, the liquid pharmaceutical formulations or unit liquid pharmaceutical formulations described herein may be used for the treatment or prevention of a variety of diseases and disorders such as, but not limited to, congestion, edema, fluid overload, and/or hypertension in a patient in need thereof.

In various embodiments, the method comprises administering to the patient a liquid pharmaceutical formulation described herein.

In various embodiments, the method comprises administering to the patient a unit liquid pharmaceutical formulation described herein.

In certain embodiments, a liquid pharmaceutical formulation described herein includes a loop diuretic such as furosemide as the sole therapeutically active agent in the liquid pharmaceutical formulation. In some embodiments, a liquid pharmaceutical formulation described herein further comprises a second therapeutic agent. In certain embodiments, the liquid pharmaceutical formulation and the second therapeutic agent are administered simultaneously, together or separately, or separately at different times, as part of a regimen.

In certain embodiments, the liquid pharmaceutical formulations described herein can be administered parenterally, including by infusion or injection, which includes subcutaneous administration as appropriate. For example, the liquid pharmaceutical formulations can be administered by, for example, subcutaneous injection or delivery, or intravenous injection or delivery. In some embodiments, the liquid pharmaceutical formulation is administered to the patient intravenously. In some embodiments, the liquid pharmaceutical formulation is administered to the patient by intramuscular injection, subcutaneous injection or subcutaneous infusion. In some embodiments, the liquid pharmaceutical formulation is administered to the patient by subcutaneous infusion using an on-body, subcutaneous delivery system. In some embodiments, the liquid pharmaceutical formulation is administered to the patient by subcutaneous infusion using a wearable subcutaneous delivery system. In some embodiments, the liquid pharmaceutical formulation is administered to the patient by subcutaneous infusion using a pump device. In some embodiments, the pump device is a micropump device or a patch device. In some embodiments, the pump device is a patch device. In some embodiments, the liquid pharmaceutical formulation is administered to the patient by intramuscular injection or subcutaneous injection using an autoinjector. In some embodiments, the autoinjector is a disposable autoinjector.

In certain embodiments, the liquid pharmaceutical formulations described herein is administered to the patient by subcutaneous infusion over about 0.5 hrs, about 1.0 hrs, about 1.5 hrs, about 2.0 hrs, about 4.0 hrs, or about 8.0 hrs. In certain embodiments, the liquid pharmaceutical formulations described herein is administered to the patient by subcutaneous infusion over about 1 minute, about 2 minutes, about 3 minutes, about 4 minutes, about 5 minutes, about 10 minutes, about 15 minutes, about 20 minutes, about 30 minutes, about 40 minutes, about 50 minutes, about 60 minutes, about 90 minutes, about 120 minutes, about 240 minutes, or about 480 minutes. In certain embodiments, the liquid pharmaceutical formulations described herein are administered to the patient by subcutaneous injection over a period of equal to or less than 30 secs (e.g., 1 sec, 2 secs, 3 secs, 4 secs, 5 secs, 6 secs, 7 secs, 8 secs, 9 secs, 10 secs, 11 secs, 12 secs, 13 secs, 14 secs, 15 secs, 16 secs, 17 secs, 18 secs, 19 secs, 20 secs, 21 secs, 22 secs, 23 secs, 24 secs, 25 secs, 26 secs, 27 secs, 28 secs, 29 secs, or 30 secs).

In certain embodiments, a unit liquid pharmaceutical formulation described herein includes a loop diuretic such as furosemide as the sole therapeutically active agent in the unit liquid pharmaceutical formulation. In some embodiments, a unit liquid pharmaceutical formulation described herein further comprises a second therapeutic agent. In some embodiments, the unit liquid pharmaceutical formulation and the second therapeutic agent are administered simultaneously, together or separately, or separately at different times, as part of a regimen.

In certain embodiments, the unit liquid pharmaceutical formulations described herein can be administered parenterally, including by infusion, injection or implantation, which includes subcutaneous administration as appropriate. For example, the unit liquid pharmaceutical formulations can be administered by, for example, subcutaneous injection or delivery, or intravenous injection or delivery. In some embodiments, the unit liquid pharmaceutical formulation is administered to the patient intravenously. In some embodiments, the unit liquid pharmaceutical formulation is administered to the patient by subcutaneous injection or subcutaneous infusion. In some embodiments, the unit liquid pharmaceutical formulation is administered to the patient by subcutaneous infusion using an on-body, subcutaneous delivery system. In some embodiments, the unit liquid pharmaceutical formulation is administered to the patient by subcutaneous infusion using a wearable subcutaneous delivery system. In certain embodiments, the unit liquid pharmaceutical formulation is administered to the patient by subcutaneous infusion using a pump device. In some embodiments, the pump device is a micropump device or a patch device. In some embodiments, the pump device is a patch device.

In certain embodiments, the unit liquid pharmaceutical formulations described herein are administered to the patient by subcutaneous infusion over about 0.5 hrs, about 1 hrs, about 1.5 hrs, about 2 hrs, about 4 hrs, or about 8 hrs. In certain embodiments, the unit liquid pharmaceutical formulations described herein are administered to the patient by subcutaneous infusion over a period of equal to or less than 30 secs.

When administered for the treatment or prevention of a disease or disorder disclosed herein, it may be understood that an effective dosage can vary depending upon many factors such as the particular compound or therapeutic combination utilized, the mode of administration, and severity of the condition being treated, as well as the various physical factors related to the individual being treated. In therapeutic applications, a liquid pharmaceutical formulation or unit liquid pharmaceutical formulation described herein may be provided to a patient already suffering from said disease or disorder in an amount sufficient to cure or at least partially ameliorate the symptoms of the disease or disorder and its complications. The dosage to be used in the treatment of a specific individual typically must be subjectively determined by an attending physician. The variables involved include the specific condition and state as well as the size, age and response pattern of the patient. In some embodiments, the amount sufficient to cure or at least partially ameliorate the symptoms of the disease or disorder and its complications is an effective amount. In some embodiments, the amount sufficient to cure or at least partially ameliorate the symptoms of the disease or disorder and its complications is a therapeutically effective amount.

### Kits

In one aspect, the invention provides kits for the treatment or prevention of a variety of diseases and disorders such as, but not limited to, congestion, edema, fluid overload, or hypertension in a patient in need thereof.

In various embodiments, a kit comprises a liquid pharmaceutical formulation described herein. In certain embodiments, the kit comprises one or more unit doses of the liquid pharmaceutical formulation. In certain embodiments, the kit further comprises a medical device. In certain embodiments, the kit further comprises instructions for treating a disease or disorder of the present invention.

In various embodiments, a kit comprises a unit liquid pharmaceutical formulation described herein. In certain embodiments, the kit comprises one or more units of the liquid pharmaceutical formulation. In some embodiments, the kit comprises one, two, three or more units of the liquid pharmaceutical formulation. In certain embodiments, the kit further comprises a medical device. In certain embodiments, the kit further comprises instructions for treating a disease or disorder of the present invention.

A number of medical devices have been proposed to facilitate self-administration of a pharmaceutical formulation. The device may include a reservoir containing, for example, pre-loaded with, the liquid pharmaceutical formulation described herein to be administered. For example, a micropump can provide precise subcutaneous administration of small quantities of a liquid pharmaceutical formulation. Such micropumps can be compact and portable. Another type of device useful for subcutaneous delivery or administration of pharmaceutical formulations is often referred to as a patch device, an on-body, subcutaneous delivery system, or a wearable subcutaneous delivery system (e.g., a pump-patch device). Patch devices usually are attached directly to the skin of a patient. A type of device useful for the intramuscular or subcutaneous delivery of pharmaceutical formulations is an autoinjector.

Accordingly, in various embodiments, a medical device such as a micropump or patch device can include a reservoir containing a pharmaceutical formulation, a subcutaneous injection needle configured for removable insertion into skin of a patient, a micropump having an inlet in fluid communication with the reservoir and an outlet in fluid communication with the subcutaneous injection needle, a control system configured for controlling the micropump to deliver the pharmaceutical formulation from the reservoir to the subcutaneous injection needle, whereby the pharmaceutical formulation is administered subcutaneously to a patient, and a housing for supporting the reservoir, subcutaneous injection needle, micropump and control system, the housing being portable and adapted for contact with the skin of the patient. The liquid pharmaceutical formulation contained within the reservoir can be any of the liquid pharmaceutical formulations or the unit liquid pharmaceutical formulations described herein.

In certain embodiments, the medical device can be of a unitary construction. Such medical devices can be for a single or one-time use. In particular embodiments, the medical device can be of a multi-piece construction. In such medical devices, a disposable or a reusable portion or component can be present. For example, a housing defining or including the reservoir can be a disposable or a reusable component of the medical device. In some embodiments, the disposable or reusable housing defining or including the reservoir can contain a pharmaceutical formulation of the present teachings. In various embodiments, the subcutaneous injection needle can be a disposable component of the medical device.

In certain embodiments, the medical device is a pump device. In some embodiments, the pump device is a micropump device or a patch device. In some embodiments, the pump device is a patch device. In certain embodiments, the medical device is selected from the group comprising a needle and syringe set, an autoinjector, a single use fixed dose injection pen, a multiuse fixed dose injection pen, a single use variable dose injection pen, or a multiuse variable dose injection pen.

### EXAMPLES

The invention now being generally described, will be more readily understood by reference to the following examples, which are merely for the purposes of illustration of certain aspects and embodiments of the present invention, and are not intended to limit the invention.

### Example 1. Manufacturing Process for Furosemide Liquid Pharmaceutical Formulations

In the following example, the manufacturing process is outlined for all exemplified furosemide liquid pharmaceutical formulations.

The corresponding amounts of the ingredients are provided in the formulas disclosed in Table 1.

Tromethamine and Sodium Metabisulfite were added to an appropriate amount of water for injection (WFI) and mixed until dissolved. Sodium hydroxide (NaOH) was added. Approximately ⅓ of total amount of drug substance, furosemide, was added and mixed until dissolved. Benzyl alcohol was added and mixed until homogenous. NaOH was added. Approximately ⅓ of total amount of drug substance, furosemide, was added and mixed until dissolved. NaOH was added. The remainder of drug substance, furosemide, was added and mixed until dissolved. A pH sample was taken and the solution adjusted with additional NaOH to a pH of 7.20 to 7.60. After pH adjustment, WFI was added to the resulting solution to achieve the final weight and mixed. The solution was filtered through a 0.22 μm PVDF filter and filled into glass syringes.

TABLE 1
Formula for Furosemide Liquid Pharmaceutical Formulations

| | Formulation | |
|---|---|---|
| Component | (pH = 6.5-8.5) | |
| Furosemide | 60-120 | mg/mL |
| Tris Buffer | 50-125 | mM |
| Benzyl Alcohol | 1-3.5% | (w/w) |
| Sodium Metabisulfite | 0.05-2% | (w/w) |

| | |
|---|---|
| Sodium Chloride | Varied mg/mL |
| Hydrochloric Acid | qs |
| Sodium Hydroxide | qs |
| Water | |

### INCORPORATION BY REFERENCE

The entire disclosure of each of the patent documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Scope of the disclosure is thus indicated by the appended claims rather than by the foregoing description, and all changes that come within the meaning and range of equivalency of the claims are intended to be embraced therein.

1. A liquid pharmaceutical formulation comprising:
(i) furosemide, or a pharmaceutically acceptable salt thereof, in a therapeutically effective amount;
(ii) an antioxidant; and
(iii) a pharmaceutically acceptable buffer.

2. The liquid pharmaceutical formulation of claim 0, further comprising:
(iv) one or more additional pharmaceutically acceptable excipients.

3. The liquid pharmaceutical formulation of claim 2, wherein the one or more additional pharmaceutically acceptable excipients is selected from the group consisting of ethanol, benzyl alcohol, glycerin, N-methyl-pyrrolidone (NMP), sodium chloride, sodium carbonate, mannitol, lactose, dextrose, a polyethylene glycol (PEG), propylene glycol, a polysorbate, a polyvinylpyrrolidone (PVP), a cyclodextrin or a derivative thereof, vitamin E or a derivative thereof, and any combination thereof.

4. The liquid pharmaceutical formulation of claim 2, wherein the one or more additional pharmaceutically acceptable excipients comprises benzyl alcohol.

5. The liquid pharmaceutical formulation of claim 4, wherein the amount of benzyl alcohol in the liquid pharmaceutical formulation is from about 0.1% (w/w) to about 10% (w/w).

6. The liquid pharmaceutical formulation of claim 1, wherein the concentration of the pharmaceutically acceptable buffer in the liquid pharmaceutical formulation is from about 10 mM to about 500 mM.

7. The liquid pharmaceutical formulation of claim 1, wherein the pharmaceutically acceptable buffer comprises a buffering agent selected from the group consisting of histidine, a citrate salt, sodium phosphate, potassium phosphate, tromethamine or a pharmaceutically acceptable salt thereof, and any combination thereof.

8. The liquid pharmaceutical formulation of claim 7, wherein the buffering agent is tromethamine or a pharmaceutically acceptable salt thereof.

9. The liquid pharmaceutical formulation of claim 8, wherein the concentration of tromethamine or a pharmaceutically acceptable salt thereof in the liquid pharmaceutical formulation is from about 10 mM to about 200 mM.

10. The liquid pharmaceutical formulation of claim 8, wherein the concentration of tromethamine or a pharmaceutically acceptable salt thereof in the liquid pharmaceutical formulation is from about 25 mM to about 150 mM.

11. The liquid pharmaceutical formulation of claim 1, wherein the amount of the antioxidant in the liquid pharmaceutical formulation is from about 0.05% (w/w) to about 2% (w/w).

12. The liquid pharmaceutical formulation of claim 1, wherein the antioxidant is selected from the group consisting of sodium metabisulfite, sodium bisulfite monothioglycerol, methionine, EDTA, and combinations thereof.

13. The liquid pharmaceutical formulation of claim 1, wherein the concentration of furosemide in the liquid pharmaceutical formulation is from about 1 mg/mL to about 250 mg/mL.

14. The liquid pharmaceutical formulation of claim 1, wherein the pH of the liquid pharmaceutical formulation is from about 6.5 to about 8.5.

15. (canceled)

16. A unit liquid pharmaceutical formulation comprising:
(i) from about 1 mg to about 250 mg of furosemide, or a pharmaceutically acceptable salt thereof;
(ii) from about 0.1% (w/w) to about 30% (w/w) of one or more additional pharmaceutically acceptable excipients selected from the group consisting of ethanol, benzyl alcohol, glycerin, N-methyl-pyrrolidone (NMP), sodium chloride, sodium carbonate, mannitol, lactose, dextrose, a polyethylene glycol (PEG), propylene glycol, a polysorbate, a polyvinylpyrrolidone (PVP), a cyclodextrin or a derivative thereof, vitamin E or a derivative thereof, and any combination thereof;
(iii) from about 0.05% (w/w) to about 2% (w/w) of an antioxidant selected from the group consisting of sodium metabisulfite, sodium bisulfite monothioglycerol, methionine, EDTA, and combinations thereof; and
(iv) from about 10 mM to about 500 mM of a pharmaceutically acceptable buffer selected from the group consisting of histidine, a citrate salt, sodium phosphate, potassium phosphate, tromethamine or a pharmaceutically acceptable salt thereof, and any combination thereof,
wherein the pH of the liquid pharmaceutical formulation is from about 6.5 to about 8.5.

17. The liquid pharmaceutical formulation of claim 1 further comprising one of more of water; a pH adjuster; and an osmolarity adjuster.

18. The liquid pharmaceutical formulation of claim 17, wherein the pH adjuster is selected from the group consisting of potassium hydroxide, sodium hydroxide, hydrochloric acid, and combinations thereof; and the osmolarity adjuster is selected from the group consisting of sodium chloride, potassium chloride, and combinations thereof.

19. A method of treating congestion, edema, fluid overload, or hypertension in a patient in need thereof, the method comprising administering to the patient the liquid pharmaceutical formulation of claim 1.

20. The method of claim 19, wherein the liquid pharmaceutical formulation is administered to the patient by subcutaneous injection or subcutaneous infusion.

21. The method of claim 20, wherein the liquid pharmaceutical formulation is administered to the patient by subcutaneous infusion using an on-body, subcutaneous delivery system.

22. The method of claim 21, wherein the on-body, subcutaneous delivery system is a pump device.

23-25. (canceled)

26. A kit for the treatment of congestion, edema, fluid overload, or hypertension comprising a liquid pharmaceutical formulation of claim 1.

27-29. (canceled)
